SEQUENCE LISTING (5 sequences)

SEQ ID NO: 1 - DNA, 20 bp, Homo sapiens
ggtgtctaggtgctccaggt

SEQ ID NO: 2 - DNA, 20 bp, Homo sapiens
gcactctccaggaggacaca

SEQ ID NO: 3 - DNA, 18 bp, Homo sapiens
cacaccgcgcctggtacc

SEQ ID NO: 4 - skipped

SEQ ID NO: 5 - DNA, 18 bp, Homo sapiens
ccgctgcctgcaaagtcc

(19) United States
(12) Patent Application Publication
(10) Pub. No.: US 2010/0075926 A1
(43) Pub. Date: Mar. 25, 2010

(54) ACTIVATION OF HISTONE DEACETYLASE 1 (HDAC1) PROTECTS AGAINST DNA DAMAGE AND INCREASES NEURONAL SURVIVAL

(76) Inventors: Li-Huei Tsai, Cambridge, MA (US); Stephen J. Haggarty, Dorchester, MA (US); Dohoon Kim, Somerville, MA (US)

Correspondence Address: WOLF GREENFIELD & SACKS, P.C., 600 ATLANTIC AVENUE, BOSTON, MA, 02210-2206, US

(21) Appl. No.: 12/508,481

(22) Filed: Jul. 23, 2009

Related U.S. Application Data
(60) Provisional application No. 61/135,716, filed on Jul. 23, 2008.

(51) Int. Cl. A61K 31/675; A61K 31/663; A61K 31/662; A61K 31/55; A61P 9/10; A61K 31/352; A61K 31/195; A61K 31/16; A61K 31/138; C07D 223/16; C07D 311/78; C07D 311/02; C07C 229/28; C07C 237/06; C07C 215/20; C07F 9/02; C07F 9/28

(52) U.S. Cl. 514/82; 514/89; 514/108; 514/114; 514/217.02; 514/453; 514/456; 514/567; 514/616; 514/653; 540/595; 549/381; 549/403; 562/444; 564/153; 564/355; 568/14; 568/15

(57) ABSTRACT

The invention provides methods and compounds for the treatment of neurological disorders, including Alzheimer's disease, Parkinson's disease, Huntington's disease, ALS (Amyotrophic Lateral Sclerosis), traumatic brain injury, ischemic brain injury or a stroke. In one aspect the compounds are HDAC1 activators. Exemplary HDAC1 activators include metal chelators, iron chelators, deferoxamin, flavonoids, compounds comprising a catechol moity, ginkgetin K, Chembridge 5104434, sciadopilysin, tetrahydrogamboic acid, TAM-11, LY 235959, CGS 19755, SK&F 97541, etidronic acid, levonordefrin, methyldopa, ampicillin trihydrate, D-aspartic acid, gamma-D-glutamylaminomethylsulfonic acid, phenazopyridine to hydrochloride, oxalamine citrate salt, podophyllotoxin, SK&F 97541, (+-)-4-amino-3-(5-chloro-2-thienyl)-butanoic acid, (RS)-(tetrazol-5-yl) glycine, R(+)-SKF-81297, gambogic acid, and derivatives thereof.

### RELATED APPLICATIONS

The present application claims priority under 35 U.S.C. §119(e) to U.S. provisional patent application, U.S. Ser. No. 61/135,716, files Jul. 23, 2008, which is incorporated herein by reference.

### GOVERNMENT FUNDING

Research leading to various aspects of the present invention were sponsored, at least in part, by NINDS grant PO1-Project 2 (AG027916). Accordingly, the U.S. Government has certain rights in the invention.

### FIELD OF THE INVENTION

The field of the invention pertains to the activation of histone deacetylases and the treatment of neurological disorders.

### BACKGROUND OF THE INVENTION

In a variety of neurodegenerative disorders such as ischemia and Alzheimer's disease (Hayashi et al., 2000; Rashidian et al., 2007; Vincent et al., 1996; Yang et al., 2001), neurons engage in aberrant cell cycle activities, expressing cell cycle markers such as Ki-67 and PCNA, and undergoing a limited extent of DNA replication (Yang et al., 2001). This behavior is remarkable considering that neurons have terminally differentiated during development and remain quiescent for decades prior to the onset of these events. While the underlying mechanisms are poorly understood, multiple lines of evidence suggest that these activities play an early and contributory role in neuronal death (Andorfer et al., 2005; Busser et al., 1998; Herrup and Busser, 1995; Nguyen et al., 2002). For example, overexpression of cell cycle activity-inducing proteins such as SV40 large T antigen, c-myc, c-Myb, or E2F-1 causes neuronal death in vitro and in vivo (al-Ubaidi et al., 1992; Konishi and Bonni, 2003; Liu and Greene, 2001; McShea et al., 2006), while pharmacological inhibitors of CDKs or other cell cycle components can exert neuroprotective effects (Padmanabhan et al., 1999).

DNA damage may also be involved in multiple conditions involving neuronal death (Adamec et al., 1999; Ferrante et al., 1997; Hayashi et al., 1999; Kruman et al., 2004; Robison and Bradley, 1984). For example, oxidative damage to neuronal DNA has been observed in rodent models of ischemia (Hayashi et al., 1999). Accumulation of reactive oxygen species results in DNA damage, cell cycle activity, and neurodegeneration in mutant mice with disrupted apoptosis-inducing factor (AIF)(Klein et al., 2002). In addition, congenital syndromes with DNA repair gene mutations, such as ataxia telangiectasia and Werner's syndrome, display a progressive neurodegeneration phenotype, demonstrating the importance of maintaining DNA integrity in the adult brain (Rolig and McKinnon, 2000). Importantly, DNA damage is involved in the aging of the human brain (Lu et al., 2004), which suggests that DNA damage may play a role in age-dependent neurological disorders as well.

A need remains for new compounds and treatment options that result in the protection of cells, including neuronal cells to DNA damage. The suppression of DNA damage in neuronal cells is an important mechanism for suppressing neuronal cell death and provides an opportunity for the treatment and prevention of neurological disorders.

### SUMMARY OF THE INVENTION

In one aspect, the invention provides methods and compositions for the suppression of DNA damage in neuronal cells and the treatment of neurological disorders.

In one aspect, the invention provides a method for treating a neurological disorder in a subject, the method comprising administering to a subject in need of treatment for a neurological disorder a therapeutically effective amount of an HDAC1 (Histone deacetylase 1) activator to treat the neurological disorder. In some embodiments the neurological disorder is Alzheimer's disease, Parkinson's disease, Huntington's disease, ALS (Amyotrophic Lateral Sclerosis), traumatic brain injury, or ischemic brain injury. In some embodiments the HDAC1 activator is a metal chelator. In some embodiments the HDAC1 activator is an iron chelator. In some embodiments the iron chelator is deferoxamine. In some embodiments the HDAC1 activator is a flavonoid. In certain embodiments the HDAC1 activator includes a catechol moity. In some embodiments the flavonoid is ginkgetin K. In some embodiments the HDAC1 activator is Chembridge 5104434, sciadopilysin, tetrahydrogamboic acid, TAM-11, gambogic acid, or a derivative thereof. In certain embodiments, the compound is LY 235959, CGS 19755, SK&F97541, or etidronic acid. In certain embodiments, the compound is levonordefrin, methyldopa, ampicillin trihydrate, D-aspartic acid, gamma-D-glutamylaminomethylsulfonic acid, phenazopyridine hydrochloride, oxalamine citrate salt, podophyllotoxin, SK&F97541, (+-)-4-amino-3-(5-chloro-2-thienyl)-butanoic acid, (RS)-(tetrazol-5-yl) glycine, or R(+)-SKF-81297.

In one aspect, the invention provides a method for protecting a subject against neuronal damage, the method comprising administering to a subject in need of protection against neuronal damage a therapeutically effective amount of an HDAC1 (Histone deacetylase 1) activator to protect against neuronal damage. In some embodiments the to neuronal damage is ischemic brain damage or stroke. In some embodiments the HDAC1 activator is a metal chelator. In some embodiments the HDAC1 activator is an iron chelator. In some embodiments the iron chelator is deferoxamine. In some embodiments the HDAC1 activator is a flavonoid. In certain embodiments the HDAC1 activator includes a catechol moity. In some embodiments the flavonoid is ginkgetin K. In some embodiments the HDAC1 activator is Chembridge 5104434, sciadopilysin, tetrahydrogamboic acid, TAM-11, gambogic acid, or a derivative thereof. In certain embodiments, the compound is LY 235959, CGS 19755, SK&F97541, or etidronic acid. In certain embodiments, the compound is levonordefrin, methyldopa, ampicillin trihydrate, D-aspartic acid, gamma-D-glutamylaminomethylsulfonic acid, phenazopyridine hydrochloride, oxalamine citrate salt, podophyllotoxin, SK&F97541, (+-)-4-amino-3-(5-chloro-2-thienyl)-butanoic acid, (RS)-(tetrazol-5-yl) glycine, or R(+)-SKF-81297.

In one aspect, the invention provides a method for increasing HDAC1 (Histone deacetylase 1) activity in a cell, the method comprising contacting the cell with an HDAC1 activator. In some embodiments the method comprises increasing the deacetylase activity of HDAC1. In some embodiments the method comprises increasing the expression level of HDAC1. In some embodiments the cell is in a subject. In some embodiments the HDAC1 activator is a metal chelator. In some embodiments the HDAC1 activator is an iron chelator. In some embodiments the iron chelator is deferoxamine. In some embodiments the HDAC1 activator is a flavonoid. In certain embodiments the HDAC1 activator includes a catechol moity. In some embodiments the flavonoid is ginkgetin K. In some embodiments the HDAC1 activator is Chembridge 5104434, sciadopilysin, tetrahydrogamboic acid, TAM-11, gambogic acid, or a derivative thereof. In certain embodiments, the compound is LY 235959, CGS 19755, SK&F97541, or etidronic acid. In certain embodiments, the compound is levonordefrin, methyldopa, ampicillin trihydrate, D-aspartic acid, gamma-D-glutamylaminomethylsulfonic acid, phenazopyridine hydrochloride, oxalamine citrate salt, podophyllotoxin, SK&F97541, (+-)-4-amino-3-(5-chloro-2-thienyl)-butanoic acid, (RS)-(tetrazol-5-yl) glycine, or R(+)-SKF-81297.

In another aspect, the invention provides novel compounds that are HDAC1 activators. In certain embodiments the HDAC1 activator is of the formula:

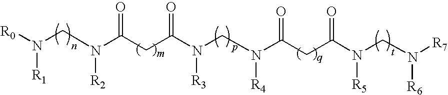

wherein
n is an integer between 1 and 6, inclusive;
m is an integer between 1 and 6, inclusive;
p is an integer between 1 and 6, inclusive;
q is an integer between 1 and 6, inclusive;
t is an integer between 1 and 6, inclusive;
R₀ is hydrogen, hydroxyl, acyl, or a nitrogen protecting group;
R₁ is hydrogen, hydroxyl, acyl, or a nitrogen protecting group;
R₂ is hydrogen, hydroxyl, acyl, or a nitrogen protecting group;
R₃ is hydrogen, hydroxyl, acyl, or a nitrogen protecting group;
R₄ is hydrogen, hydroxyl, acyl, or a nitrogen protecting group;
R₅ is hydrogen, hydroxyl, acyl, or a nitrogen protecting group;
R₆ is hydrogen, hydroxyl, acyl, or a nitrogen protecting group;
   R₇ is hydrogen, hydroxyl, acyl, or a nitrogen protecting group; and a pharmaceutically acceptable salt thereof.

In certain embodiments, the HDAC1 activator is of the formula:

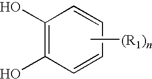

wherein
n is an integer between 1 and 4, inclusive;
each of R₁ is independently hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{A}; —C(═O)R_{A}; —CO₂R_{A}; —CN; —SCN; —SR_{A}; —SOR_{A}; —SO₂R_{A}; —NO₂; —N₃; —N(R_{A})₂; —NHC(═O)R_{A}; —NR_{A}C(═O)N(R_{A})₂; —OC(═O)OR_{A}; —OC(═O)R_{A}; —OC(═O)N(R_{A})₂; —NR_{A}C(═O)OR_{A}; or —C(R_{A})₃; wherein each occurrence of R_{A} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety; and pharmaceutically acceptable salts thereof.

In certain embodiments, the HDAC1 activator is of the formula:

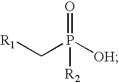

wherein
R₁ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{A}; —C(═O)R_{A}; —CO₂R_{A}; —CN; —SCN; —SR_{A}; —SOR_{A}; —SOR_{A}; —SO₂R_{A}; —NO₂; —N₃; —N(R_{A})₂; —NHC(═O)R_{A}; —NR_{A}C(═O)N(R_{A})₂; —OC(═O)OR_{A}; —OC(═O)R_{A}; ——OC(═O)N(R_{A})₂; —NR_{A}C(═O)OR_{A}; or —C(R_{A})₃; wherein each occurrence of R_{A} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;
R₂ is cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{B}; —OH; or —C(R_{B})₃; wherein each occurrence of R_{A} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety; and pharmaceutically acceptable salts thereof.

In certain embodiments, the HDAC1 activator is of the formula:

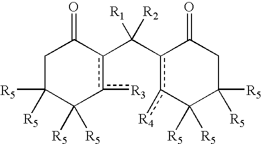

wherein

each [symbol] is independently a single or double bond;

each of R₁ and R₂ is independently hydrogen; cyclic or acyclic, branched or unbranched, substituted or unsubstituted aliphatic; cyclic or acyclic, substituted or unsubstituted, branched or unbranched heteroaliphatic; substituted or unsubstituted, branched or unbranched acyl; substituted or unsubstituted aryl, substituted or unsubstituted, branched or unbranched heteroaryl; —OR_{A}; —C(═O)R_{A}; —CO₂R_{A}; —CN; —SCN; —SR_{A}; —SOR_{A}; —SO₂R_{A}; —NO₂; —N₃; —N(R_{A})₂; —NHC(═O)R_{A}; —NR_{A}C(═O)N(R_{A})₂; —OC(═O)OR_{A}; —OC(═O)R_{A}; —OC(═O)N(R_{A})₂; —NR_{A}C(═O)OR_{A}; or —C(R_{A})₃; wherein each occurence of R_{A} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

each of R₃, and R₄ is independently —OH, alkoxy, —Oacyl, ═O, or wherein R₃ and R₄ are taken together to form a cyclic structure;

each of R₅ is independently hydrogen; cyclic or acyclic, branched or unbranched, substituted or unsubstituted aliphatic; and pharmaceutically acceptable salts thereof.

In certain embodiments, the HDAC1 activator is of the formula:

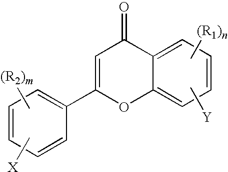

wherein
n is an integer between 0 and 4, inclusive;
m is an integer between 0 and 5, inclusive;

each of R₁ and R₂ is independently —OH; alkoxy; —Oacyl; —OAc; —OP_{G}; substituted or unsubstituted aryl;

wherein either R₁ or R₂ can be a second HDAC1 activator moiety; and pharmaceutically acceptable salts thereof.

to In certain embodiments, the HDAC1 activator is of the formula:

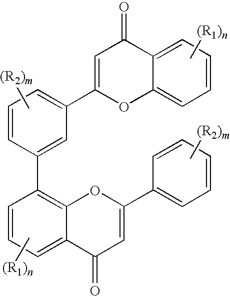

wherein
n is an integer between 0 and 4, inclusive;
m is an integer between 0 and 4, inclusive;

each of R₁ and R₂ is independently —OH; alkoxy; —Oacyl; —OAc; —OP_{G}; substituted or unsubstituted aryl; and pharmaceutically acceptable salts thereof.

In certain embodiments, the HDAC1 activator is of the formula:

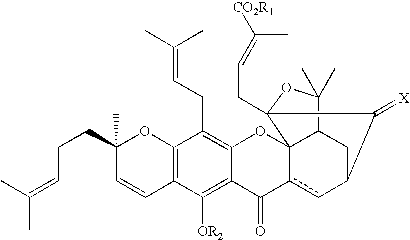

wherein

[symbol] is independently a single or double bond;

R₁ is hydrogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched aryl; substituted or unsubstituted, branched or unbranched heteroaryl;

R₂ is hydrogen; cyclic or acyclic, substituted or unsubstituted, branched or unbranched aliphatic; cyclic or acyclic, substituted or unsubstituted, branched or unbranched to heteroaliphatic; substituted or unsubstituted, branched or unbranched acyl; substituted or unsubstituted, branched or unbranched aryl; substituted or unsubstituted, branched or unbranched heteroaryl; —C(═O)R_{B}; —CO₂R_{B}; or —C(R_{B})₃; wherein each occurrence of R_{B} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

X is ═O,

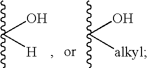

and pharmaceutically acceptable salts thereof.

In certain embodiments, the HDAC1 activator is of the formula:

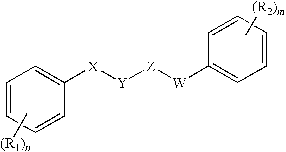

wherein

n is an integer between 0 and 5, inclusive;

m is an integer between 0 and 5, inclusive;

each X, Y, and Z is independently selected from the list consisting of CH₂, NH, C═O, and O; and

wherein W is either absent or selected from the list consisting of CH₂, NH, C═O, and O;

each of R₁ and R₂ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{A}; —C(═O)R_{A}; —CO₂R_{A}; —CN; —SCN; —SR_{A}; —SOR_{A}; —SO₂R_{A}; —NO₂; —N₃; —N(R_{A})₂; —NHC(═O)R_{A}; —NR_{A}C(═O)N(R_{A})₂; —OC(═O)OR_{A}; —OC(═O)R_{A}; —OC(═O)N(R_{A})₂; —NR_{A}C(═O)OR_{A}; or —C(R_{A})₃; wherein each occurrence of R_{A} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety; and pharmaceutically acceptable salts thereof.

In certain embodiments, the HDAC1 activator is of the formula:

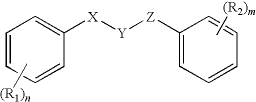

wherein

n is an integer between 0 and 5, inclusive;

m is an integer between 0 and 5, inclusive;

each X, Y, and Z is independently selected from the list consisting of CH₂, NH, C═O, O, and S;

each of R₁ and R₂ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{A}; —C(═O)R_{A}; —CO₂R_{A}; —CN; —SCN; —SR_{A}; —SOR_{A}; —SO₂R_{A}; —NO₂; —N₃; —N(R_{A})₂; —NHC(═O)R_{A}; —NR_{A}C(═O)N(R_{A})₂—; —OC(═O)OR_{A}; —OC(═O)R_{A}; —OC(═O)N(R_{A})₂; —NR_{A}C(═O)OR_{A}; or —C(R_{A})₃; wherein each occurrence of R_{A} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety; and pharmaceutically acceptable salts thereof.

In certain embodiments, the invention provides pharmaceutical compositions comprising one of the above-mentioned compounds and a pharmaceutically acceptable excipient. In certain embodiments, the pharmaceutical composition comprises a therapeutically effective amount of an HDAC1 activator as described herein.

In one aspect, the invention provides a kit for treating a neurological disorder comprising a first container comprising a HDAC1 (Histone deacetylase 1) activator and instructions for administering the HDAC1 activator to a subject to treat a neurological disorder. In some embodiments the neurological disorder is Alzheimer's disease, Parkinson's disease, Huntington's disease, ALS (Amyotrophic Lateral Sclerosis), traumatic brain injury, ischemic brain injury. In some embodiments the HDAC1 activator is a metal chelator. In some embodiments the HDAC1 activator is an iron chelator. In some embodiments the iron chelator is deferoxamine. In some embodiments the HDAC1 activator is a flavonoid. In certain embodiments the HDAC1 activator includes a catechol moity. In some embodiments the flavonoid is ginkgetin K. In some embodiments the HDAC1 activator is Chembridge 5104434, sciadopilysin, tetrahydrogamboic acid, TAM-11, gambogic acid, or a derivative thereof. In certain embodiments, the compound is LY 235959, CGS 19755, SK&F97541, or etidronic acid. In certain embodiments, the compound is levonordefrin, methyldopa, ampicillin trihydrate, D-aspartic acid, gamma-D-glutamylaminomethylsulfonic acid, phenazopyridine hydrochloride, oxalamine citrate salt, podophyllotoxin, SK&F97541, (+-)-4-amino-3-(5-chloro-2-thienyl)-butanoic acid, (RS)-(tetrazol-5-yl) glycine, or R(+)-SKF-81297.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including”, “comprising”, “having”, “containing”, “involving”, and variations thereof herein, is meant to encompass the items listed thereafter and equivalents thereof as well as additional items.

### BRIEF DESCRIPTION OF THE DRAWINGS

The figures are illustrative only and are not required for enablement of the invention disclosed herein.

FIG. 1 shows that cell cycle markers are aberrantly upregulated following p25 induction. (A) 2-week induced CK-p25 mice and WT controls were analyzed for PCNA, cyclinA, and E2F-1 protein levels. Glial fibrillary acidic protein (GFAP), or BetaIII-tubulin, used as loading control, were unchanged. (B) Ki-67, a cell cycle progression marker, is upregulated in p25 expressing neurons in CK-p25 brains (top panels), but not in neurons in WT controls (bottom panels). CA1 region is shown. (C) Proliferating cell nuclear antigen (PCNA), a proliferation/S-phase marker, is induced in p25 expressing neurons in CK-p25 brains (top panels), but not in neurons in WT controls (bottom panels). CA1 region is shown. (D) p25 expressing neurons in CK-p25 brains are not immunoreactive for the mitotic marker phospho(pS10)-Histone H3 (top panels). Subventricular zone (SVZ) of the same CK-p25 brain is shown as a positive control for mitotic cells immunoreactive for phospho-Histone H3. CA1 region is shown. Scale bar=50 μm.

FIG. 2 shows that double strand DNA damage occurs following p25 induction. (A) Western blots from induced CK-p25 mice forebrain lysates show increased levels of γH2AX and Rad51 compared to WT controls. Asterisk indicates nonspecific band. Quantification of γH2AX levels (±S.D.) from multiple WT controls (n=5) and CK-p25 mice (n=5) induced between 2 and 12 weeks are shown in top panel. (B) Staining of paraffin sections with γH2AX reveals immunoreactivity specifically in the nuclei of p25GFP-expressing neurons in two-week induced CK-p25 mice (top panels) but not in neurons of WT controls (bottom panels). CA1 region is shown. (C) Primary cortical neurons were infected with increasing titers of herpesvirus expressing p25 (p25-HSV) or lacZ-HSV control and analyzed for γH2AX protein levels by Western blot. (D) Primary cortical neurons infected with p25-HSV and fixed 8 hours post-infection display robust immunoreactivity with γH2AX (right panels), compared to control uninfected neurons (left panels). p25 overexpression was verified with p35 antibody (top panels). Top and bottom panels are from different fields. (E) Comet assays were carried out on DIV7 primary neurons infected with p25-HSV or lacZ-HSV for 10 hours, as described in Methods. Micrographs of comet assay fields are shown in the left and middle panels for p25-HSV infected and lacZ-HSV infected neurons, respectively. Comet tails indicate DNA with breaks, resulting in increased migration towards the direction of the current (left to right). Right panel shows quantification of the percentage of neurons with comet tails from three separate experiments. Results are displayed as fold change to control (lacZ-HSV infected) neurons. P-values (**p<0.005) were calculated from multiple experiments by two-tailed, unpaired Student's t-test.

FIG. 3 shows that double strand DNA breaks and aberrant cell cycle activity are concomitant and precede neuronal death. (A) Double immunofluorescence staining for Ki-67 (green) and γH2AX (red) carried out in 2 week induced CK-p25 mice revealed that cell cycle reentry and DNA double strand breaks occur concurrently in the same neurons. Representative images of CA1 region are shown in left panels, and quantification of neurons which were immunoreactive for both γH2AX and Ki-67, γH2AX only, or Ki-67 from multiple 2 week induced CK-p25 mice are shown in the histogram (a: γH2AX+Ki-67 vs. γH2AX only, p<0.001; b: γH2AX+Ki-67 vs. Ki-67 only, p<0.001. One way ANOVA with Neuman-Keuls multiple comparison test). (B) γH2AX and Ki-67 are closely associated with dying neurons at 8 weeks of p25 induction. A representative image showing association of γH2AX and Ki-67 with pyknotic nuclei (first, second, and third panels). Fourth panel is a magnification of the boxed region in third panel. Quantification of cell death (pyknotic nuclei) in p25-GFP and γH2AX immunoreactive neurons, p25-GFP and Ki-67 immunoreactive neurons, or neurons immunoreactive for p25-GFP but not γH2AX or Ki-67 are shown from multiple 2-week induced and 8-week induced CK-p25 mice (a: GFP only vs. GFP+γH2AX, p<0.01; b:GFP only vs. GFP+Ki-67, p<0.01. One way ANOVA with Neuman-Keuls multiple comparison test). (C) Primary cortical neurons at DIV 5-8 were transfected with a p25-GFP overexpression construct, fixed, and scored at various time points as shown for γH2AX immunoreactivity and for cell death, as described in Methods. Shown at left is a representative micrograph of a γH2AX immunoreactive neuron. Inset is a magnification of the γH2AX-positive nucleus. Counts are displayed as percentages of total (right). Scale bar=50 μm. (D) CK-p25 mice were induced for 2 weeks (top panels) and sacrificed, or induced for 2 weeks followed by 4 weeks of suppression through doxycyline diet prior to sacrifice. Sections were examined for GFP and γH2AX signals. It was previously determined that 2 week induction of p25 followed by 4 weeks of suppression did not result in neuronal loss (Fischer et al., 2005). Scale bar=100 μM.

FIG. 4 shows that p25 interacts with HDAC1 and inhibits its activity. (A) Forebrains from 2-week induced CK-p25 and WT control mice were homogenized and lysates immunoprecipitated with HDAC1 antibody as described in the Methods, and probed for p25-GFP and HDAC1. (B) Flag-tagged HDAC1 was overexpressed with GFP-p25 or p35 in HEK293T cells, immunoprecipitated with anti-Flag-conjugated beads as described in Methods, and probed for p25-GFP or p35-GFP. Quantification of bands reveal an over 12-fold higher affinity towards p25. (C) Flag tagged full length HDAC1 or various truncation mutations were overexpressed with GFP-p25 and immunoprecipitated with flag-conjugated beads as described. The catalytic domain is indicated in brown. (D) Left panel: HEK293T cells were transfected with vector or with p25/cdk5. After 15 hours, endogenous HDAC1 was immunoprecipitated, then assayed for histone deacetylase activity as described in the Methods. Averages from multiple experiments are displayed as fold change over control (vector only). Right panel: hippocampi from WT and CK-p25 mice were dissected and assayed for endogenous HDAC1 activity as described. P-values (“p<0.005, *p<0.05) were calculated from multiple experiments by two-tailed, unpaired Student's t-test. (E) p25/Cdk5 inhibits the transcriptional repressor activity of HDAC1. HDAC1-Ga14 construct was co-transfected with blank vector or p25/cdk5 then measured for luciferase activity as described in Methods. Values were normalized to protein levels of Ga14 constructs, and are expressed as relative light units (HDAC1-Ga14 only=1). (F) Primary cortical neurons were infected with p25-HSV or GFP-HSV then subjected to fractionation as described in the Methods. Lamin A and Histone 3 are used as markers for the nuclear and chromatin fractions, respectively. Band densitometry quantifications from multiple experiments (±S.D.) are shown in the histogram on the right. (G) HEK293T cells were transfected with blank vector or p25 and cdk5, cross-linked, then subjected to chromatin immunoprecipitation using HDAC1 antibody Immune complexes were subjected to semi-quantitative PCR amplification using primers towards the core promoter regions of E2F-1 and p21/WAF.

FIG. 5 shows that loss of HDAC1 or pharmacological inhibition of HDAC1 results in DNA damage, cell cycle reentry, and neurotoxicity. (A, B) Primary cortical neurons were transfected with either HDAC1 siRNA or random sequence siRNA, along with GFP at a 7:1 ratio to label transfected neurons. Cells were fixed at 24 h, 48 h, and 72 h post-transfection and immunostained for γH2AX. GFP-positive neurons were scored for γH2AX immunoreactivity and for cell death based on nuclear condensation and neuritic integrity, as described in Methods. (A) Representative micrographs. HDAC1 siRNA or control (random sequence) siRNA transfected neurons are indicated by arrows. The HDAC1 siRNA transfected neurons display neuritic breakage. The inset is a magnification of the γH2AX staining of the neuron indicated by arrow and asterisk, showing γH2AX foci of varying sizes. Percentage of γH2AX and cell death are shown as averages from multiple sets ±S.D. It was noted that transfection of control siRNA per se appeared to cause a low but detectable level of γH2AX immunoreactivity and cell death. (B) Primary cortical neurons were treated with 1 μM of the to HDAC1 inhibitor MS-275 for 24 h, fixed, and immunostained for γH2AX and Ki-67. Controls were treated with equal amounts of vehicle (DMSO). Total numbers of γH2AX and Ki-67 positive neurons were quantified over 20 microscope fields (field diameter ˜900 μm). Scale bar=100 μm. (C) Wild-type mice were injected intraperitoneally with 50 mg/kg MS-275 (n=3) or saline (n=3) daily for 5 days, then sacrificed and examined for γH2AX. MS-275 injection resulted in a dramatic induction of γH2AX within the CA1 (bottom panels), whereas saline injection did not induce γH2AX (top panels). Scale bar=100μM.

FIG. 6 shows that HDAC1 gain-of-function rescues against p25-mediated double strand DNA breaks and neurotoxicity. (A) Overexpression of HDAC1 rescues against p25 mediated formation of γH2AX. Primary cortical neurons at DIV6-8 were transfected with vector, HDAC1, or HDAC2 using calcium phosphate as described in the Methods. At 12 hours posttransfection, neurons were infected with p25-HSV virus, fixed after 8 hours, and immunostained for γH2AX. HDAC-positive cells were scored for immunoreactivity towards γH2AX. (B) Overexpresson of HDAC1 rescues against p25-mediated neurotoxicity. Primary cortical rat neurons at DIV6-8 were transfected with p25-GFP with or without flag-HDAC1 or flag-HDAC1-H141A mutant. At 24 h posttransfection, cells were fixed and immunostained for flag. p25(+)HDAC(+) cells and p25(+)HDAC(−) cells were scored for cell death based on nuclear condensation and neuritic integrity. For (A) and (B), averages from multiple experiments ±S.D. are shown. Representative micrographs for HDAC1 are shown on left panels. Arrows indicate p25-positive neurons expressing or not expressing HDAC1. P-values (HDAC1 vs control, “p<0.005) were calculated from multiple experiments by two-tailed, unpaired Student's t-test. Bar=50 μM. (C) Adult Sprague-Dawley rats were subjected to unilateral middle cerebral artery occlusion (MCAO) as described in the Methods. Paraffin sections from brains fixed at three hours post-MCAO show γH2AX immunoreactivity specifically within the infarct area (left panels) but not in the contralateral area (right panels). Images are representative of multiple animals. Average numbers of γH2AX-positive cells per field (field diameter ˜900 μm) from multiple experiments ±S.D. are displayed. 20 fields were counted per experiment. P-values (**p<0.005) were calculated from multiple experiments by two-tailed, unpaired Student's t-test. (D) Injection of blank vector (expressing GFP) into striatum results in efficient and widespread expression in striatal neurons. Injection of virus into the striatum of adult Sprague-Dawley rats was followed by examination of GFP expression after 24 hours. Left pane bar=100 μM, right panel bar=30 μM. (E) HDAC1 expression protects against ischemia-induced neuronal death and γH2AX formation in vivo. Adult Sprague-Dawley rats were injected with virus in the striatum, subjected to bilateral MCAO after 24 hours, then examined 6 days later for Fluoro-Jade and H2Ax staining as described in Methods. Representative images from mice injected with HSV-HDAC1, HSV-HDAC1H141A, and blank HSV (Vector) are shown. Scale bar=100 μM. (F) Quantification of γH2AX+ cells from mice injected with saline, HSV-HDAC1, HSV-HDAC1H141A, vector, or mice subjected to sham procedure are shown. (G) Quantification of FJ+ cells from the same mice as (D). For (D) and (E), data is presented as Mean ±SEM. P-values (*p<0.05; **p<0.005) were calculated from multiple experiments by two-tailed, unpaired Student's t-test. Bar=100 μM.

FIG. 7 shows a model for p25-mediated cell death involving inhibition of HDAC1 activity leading to DNA double strand breaks and aberrant cell cycle activity.

FIG. 8 shows that peritoneal administration of the HDAC1 inhibitor MS-275 induces cognitive impairment. WT mice were subjected to IP injection daily for 10 days with saline (n=20) or MS-275 (12.5 mg/kg, n=8; or 25mg/kg, n=6), then were subjected to contextual fear conditioning. Mice treated with 25 mg/kg MS-275 displayed reduced freezing behavior, suggesting a loss of associative learning. *p=0.013; two-tailed, unpaired Student's t-test.

FIG. 9 shows the results of a high-throughput screen of 1,760 compounds (colored circles) for selective activators of the deacetylase activity of HDAC1. Values indicate % deacetylase inhibition (avg. n=2) relative to a solvent (DMSO) control treatment measured using recombinant human HDAC1 or HDAC2 and Caliper's mobility shift assay technology. Circle color corresponds to compounds shaded by degree of HDAC1 activity (red, decreased; blue, increased). (A) Complete dataset with box outlined with the red dashes corresponding to the region shown highlighted in (B), which in the assay corresponds to negative inhibition. Other compounds were found to be selective activators of others HDACs but not HDAC1 (e.g., 5122155 for HDAC2) highlighting the specificity of the assays.

FIG. 10 shows that expression of HDAC1 ameliorates p25-induced neurotoxicity. Primary cortical neurons at DIV 5-7 were transfected with p25 plus blank vector or various HDACs as shown. At 24 h posttransfection, cells were fixed and immunostained for flag. p25(+)HDAC1(+) cells were scored for cell death based on nuclear condensation and neuritic integrity. Averages from multiple experiments (±S.D.) are shown where available. P-values to (HDAC1 vs control, **p<0.005) were calculated from multiple experiments by two-tailed, unpaired Student's t-test. Representative images from p25 cotransfected with HDAC1 is shown in top panels. Arrows indicate p25 positive cells; in the micrographs, it is observed that cells that are positive for p25 and HDAC1, have a normal nonapoptotic morphology, while cells only positive for p25 have lost neuritic integrity (indicated by neuritic blebbing). Scale bar=50 μM.

FIG. 11A, B shows the chemical structures of selected HDAC1 activators.

FIG. 12 shows the chemical structures of selected HDAC1 activators.

### DETAILED DESCRIPTION OF THE INVENTION

In one aspect, the invention provides methods and compositions for the treatment of neurological disorders. In some embodiments neurological disorders are treated by decreasing the amount of DNA damage within the neuronal cell. In some embodiments neurological disorders are treated by increasing histone deacetylase activity within the neuronal cell. In some embodiments neurological disorders are treated by decreasing histone acetyl transferase activity within the neuronal cell. In some embodiments neurological disorders are treated by increasing the activity of class I histone deacetylases. In some embodiments neurological disorders are treated by increasing the activity of HDAC1.

Regulating histone acetylation is an integral aspect of chromatin modulation and gene regulation that plays a critical role in many biological processes including cell proliferation and differentiation (Roth et al., 2001). Recent reports have detailed the importance of histone acetylation in CNS functions such as neuronal differentiation, memory formation, drug addiction, and depression (Citrome, 2003; Johannessen and Johannessen, 2003; Tsankova et al., 2006). Histone deacetylases (HDACs) remove acetyl groups from histones, resulting in increased chromatin compaction and decreased accessibility to DNA for interacting molecules such as transcription factors (Cerra et al., 2006). Of the HDACs, histone deacetylase 1 (HDAC1) was the first protein identified to have histone-directed deacetylase activity (Taunton et al., 1996; Vidal and Gaber, 1991). HDAC1 plays important roles in regulating the cell cycle and is required in the transcriptional repression of cell cycle genes such as p21/WAF, E2F-1, and cyclins A and E (Brehm et al., 1998; Iavarone and Massague, 1999; Lagger et al., 2002; Rayman et al., 2002; Stadler et al., 2005; Stiegler et al., 1998). The association of HDAC1 with promotor regions of specific genes is linked to their to transcriptional repression (Brehm et al., 1998; Gui et al., 2004; Iavarone and Massague, 1999; Rayman et al., 2002).

The serine/threonine kinase cdk5 and its activating subunit p35 play important roles in both the developing and adult central nervous system (Dhavan and Tsai, 2001). In numerous neurodegenerative states including postmortem Alzheimer's disease brains and animal models for stroke/ischemia (Lee et al., 2000; Nguyen et al., 2001; Patrick et al., 1999; Smith et al., 2003; Swatton et al., 2004; Wang et al., 2003), neurotoxic stimuli induce calpain mediated cleavage of p35 into p25, the accumulation of which elicits neurotoxicity in cultured neurons and in vivo (Lee et al., 2000; Patrick et al., 1999).

We have previously generated a bi-transgenic mouse model (CK-p25 mice) which expresses a p25-GFP fusion under the control of the Calmodulin Kinase II promoter in an inducible, postdevelopmental, and forebrain-specific manner (Cruz et al., 2003). Upon induction of p25, neurodegenerative events occur in a rapid and orderly manner, as astrogliosis is observed after 4 weeks of induction, and neuronal loss and cognitive impairment is appreciable after 6 weeks of induction (Cruz et al., 2003; Fischer et al., 2005). Thus, this model provides a tractable system for investigating mechanisms for neuronal death relevant to multiple neurodegenerative conditions which involve p25, including stroke/ischemia and Alzheimer's disease.

We examined the gene expression profile in p25 transgenic mice which were induced for a short period, to gain insights into early and instigating mechanisms involved in neurodegeneration. We observed that following p25 induction, neurons aberrantly express cell cycle proteins and form double strand DNA breaks at an early stage prior to their death. p25 interacted with an inactivated HDAC1, and inactivation of HDAC1 through siRNA knockdown or pharmacological inhibition resulted in double strand DNA breaks, aberrant cell cycle protein expression, and neuronal death. Our findings show that the inactivation of HDAC1 by p25 is involved in the pathogenesis of neurological disorders. In various neurodegenerative conditions ranging from stroke/ischemia to Alzheimer's disease and Parkinson's disease, neurons display pathological features that are remarkably similar. One important pathological feature is DNA damage. Thus, decreasing the amount of DNA damage provides a method for decreasing neuronal death and/or treating neurological disorders. Restoring HDAC1 activity by overexpressing wild type HDAC1, but not the deacetylase activity-deficient mutant, rescued against p25-mediated double strand DNA to breaks and cell death. Thus, an increase in HDAC1 activity is neuroprotective.

We used a rodent ischemia model to show the neuroprotective role of HDAC1 in vivo. Lentivirus was used to express wildtype HDAC1 or a catalytically inactive HDAC1 (H141A) into the striatum of rats that were treated with the bilateral middle cerebral artery occlusion paradigm (which is a model for stroke). We found that overexpression of the wildtype but not mutant HDAC1 provided protection against ischemia induced neuronal death. Thus increased activity of HDAC1 is neuroprotective in vivo. Furthermore, the study showed that the zinc-dependent hydrolase activity of HDAC1, which catalyzes the removal of acetyl groups from the e-amino groups of lysine side chains in proteins, and not simply the presence of HDAC1, is important for neuroprotection.

Thus, agents that increase HDAC1 activity are neuroprotective and serve as agents for treatment of neurological disorders, including Alzheimer's, Parkinson's, Huntington's, Amyotrophic Lateral Sclerosis (ALS), ischemic brain damage and traumatic brain injury.

Histone deacetylases are primarily responsible for removing acetyl groups from lysine side chains in chromatin resulting in the increase of positive charge on the histone and the ability of the histone to bind DNA, resulting in the condensation of DNA structure and the prevention of transcription.

HDACs are classified in four classes depending on sequence identity, domain organization and function. Class I: HDAC1, HDAC2, HDAC3, HDAC8; Class II: HDAC4, HDAC5, HDAC6, HDAC7, HDAC9, HDAC10; Class III: SIRT1, SIRT2, SIRT3, SIRT4, SIRT5, SIRT6, SIRT7; Class IV: HDAC11. Within Class I, HDAC1, HDAC2 and HDAC8 are primarily found in the nucleus while HDAC3 and Class II HDACs can shuttle between the nucleus and the cytoplasm. Class III HDACs (the sirtuins), couple the removal of the acetyl group of the histone to NAD hydrolysis, thereby coupling the deacetylation reaction to the energy status of the cell.

Nucleosomes, the primary scaffold of chromatin folding, are dynamic macromolecular structures, influencing chromatin solution conformations. The nucleosome core is made up of histone proteins, H2A, H2B, H3 and H4. Histone acetylation causes nucleosomes and nucleosomal arrangements to behave with altered biophysical properties. The balance between activities of histone acetyl transferases (HAT) and histone deacetylases (HDAC) determines the level of histone acetylation. Acetylated histones cause relaxation of chromatin and activation of gene transcription, whereas deacetylated chromatin generally is transcriptionally inactive.

In some embodiments, neurological disorders are treated by decreasing histone acetylation by the administration of histone acetylase activators. In some embodiments neurological disorders are treated by decreasing histone acetylation by methods other than increasing HDAC activity. Methods for decreasing histone acetylation, by a method other than a classic HDAC activator include, but are not limited to, the administration of nucleic acid molecule inhibitors such as antisense and RNAi molecules which reduce the expression of histone acetyl transferases and the administration of histone acetyl transferase inhibitors. Histone acetyl transferase inhibitors are known in the art and are described for instance in Eliseeva et al. (Eliseeva E D, Valkov V, Jung M, Jung M O. Characterization of novel inhibitors of histone acetyltransferases. Mol Cancer Ther. 2007 September;6(9):2391-8). The invention embraces methods that regulate the function of any protein involved with histone modification, function and regulation.

In some embodiments, neurological disorders are treated by protecting cells from DNA damage by increasing the histone deacetylation activity within the cell. Protection from DNA damage includes both a decrease in the current level of DNA damage accumulated within the cell, or a decrease in the rate of DNA damage acquired by the cell, including DNA damage acquired in exposure of the cell to DNA damaging events, such as exposure to DNA damaging agents, including radiation, and events that lead to increased oxidative stress. Increased deacetylase activity can protect against any form of DNA damage, including base modifications, DNA single strand breaks and DNA double strand breaks. DNA double strand breaks are potentially the most damaging to the cell, and other forms of DNA damage can be turned into DNA double strand breaks by the action of DNA repair enzymes and other cellular processes. DNA damage, including DNA double strand breaks can accumulate in both actively dividing and non-dividing cells. In actively dividing cells, DNA double strand breaks may inhibit the replication machinery, while in both actively dividing and non-dividing cells the transcription machinery may be inhibited by DNA double strand breaks. In addition DNA double strand breaks may initiate potentially damaging recombination events. Thus, increased deacetylase activity may be protective in any cell type, including dividing and non-dividing cells. In some embodiments increased deacetylase activity is protective in neuronal cells. In some embodiments increased deacetylase activity is induced in cells that are susceptible to acquiring DNA damage, or cells that will be subjected to a DNA damage inducing event. For instance histone deacetylase activity may be increased in cells or tissue in a subject that need to be protected when a DNA damaging agent is administered throughout the body (for instance during chemotherapy). In some embodiments neuroprotection is provided by increasing the histone deacetylation activity within a neuronal cell. In some embodiments neuroprotection is provided by decreasing the histone acetyl transferase activity within a neuronal cell.

The invention embraces any method of increasing deacetylase activity. In some embodiments deacetylase activity is increased by increasing the activity of HDAC1. In some embodiments deacetylase activity is increased by adding an HDAC activator to the cell. In some embodiments the HDAC activator is an HDAC1 activator. In some embodiments HDAC activity is increased by increasing the expression level of one or more HDACs. In some embodiments HDAC activity is increased by selectively increasing the expression level of one or more HDACs relative to one or more HDACs. In some embodiments HDAC activity is increased by selectively increasing the expression level of one or more HDACs by 1%, 2%, 3%, 4%, 5%, 6%, 7%, 8%, 9%, 10%, 11%, 12%, 13%, 14% 15%, 16%. 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, to 60%, 60% to 70%, 70% to 80%, 80% to 90%, or 90% to 100% relative to one or more HDACs. In some embodiments HDAC activity is increased by selectively increasing the expression level of one or more HDACs by 100% to 200%, 200% to 300%, 300% to 500%, 500% to 1000%, 1000% to 10000%, or 10000% to 100000% relative to one or more HDACs. In some embodiments the expression level is increased by increasing the level and/or activity of transcription factors that act on a specific gene encoding a histone deacetylase. In some embodiments the activity is increased by decreasing the activity of repressor elements. In some embodiments deacetylase activity within a cell or subject is increased by administering histone deacetylase protein to the cell or subject. In some embodiments the activity is increased by inactivating or sequestering an agent that acts as an inhibitor on a HDAC suppressor pathway.

An “HDAC activator” as defined herein is any compound that results in an increase in the level of HDAC activity. Any increase in enzymatic function by HDAC is embraced by the invention. In some embodiments the activity increase of HDAC is an increase in HDAC deacetylase activity. In some embodiments the activity increase of HDAC is an increase in to HDAC esterase activity. HDAC activity corresponds to the level of histone deacetylase activity of the HDAC. One of ordinary skill in the art can select suitable compounds on the basis of the known structures of histone deacetylases. Examples of such compounds are peptides, nucleic acids expressing such peptides, small molecules etc, each of which can be naturally occurring molecules, synthetic molecules and/or FDA approved molecules, that specifically react with the histone deacetylase and increase its activity.

In some embodiments, the HDAC activator is a naturally occurring compound or derivative thereof such as flavonoid. Flavonoids are plant secondary metabolites with a core phenylbenzyl pyrone structure, and include the subclasses of flavones, isoflavones, neflavones flavonols, flavanones, flavan-3-ols, catechins, anthocyanidins and chalcones. Non-limiting examples of flavonoids are epicatechin, quercetin, luteolin, epicatechin, proanthocyanidins, hesperidin, tangeritin, ginkgetin K, kaempferol, catechins (including catechin, epicatechin, epicatechin gallate, and epigallocatechin gallate), apigenin, myricetin, fisetin, isorhamnetin, pachypodol, rhamnazin, hesperetin, naringenin, eriodictyol, taxifolin, cyanidin, delphinidin, malvidin, pelargonidin, peonidin and petunidin. Examples of flavonoids suitable for use in the present invention include those listed in U.S. Pat. No. 7,410,659, the entirety of which is incorporated herein by reference.

In some embodiments, the HDAC activator is a gambogic acid or derivatives thereof. Examples of gambogic acid derivatives suitable for use in the present invention include those listed in U.S. Pat. No. 6,613,762, the entirety of which is incorporated herein by reference.

In some embodiments, the HDAC activator is a metal chelator. Chelators include both small molecules and proteins. Chelators are molecules that bind metal ions. Non-limiting examples of chelators are ethylene diamine, tetra acetic acid, EDTA, hydroxylamines and N-substituted hydroxylamines, deferoxamin (also known as desferrioxamine, desferoxamin and desferal) and transferrin. All chelators bind metal ions in inert fashion. Some chelators are specific to a certain metal ion, such as iron, while other chelators can bind any metal ion. In some embodiments the HDAC activator is a iron chelator. Chelators can be used to remove metal ions and prevent poisoning and the accumulation of excess metal ions in a subject. For example, the iron chelator, desferrioxamine, is used to remove excess iron that accumulates with chronic blood transfusions.

In some embodiments, the HDAC activator is a chromone derivative, chromanone derivative, benzoxazole derivative, indole derivative, sulfonic acid derivative, benzoic acid derivative, xanthene-1,8-dione derivative, analine derivative, 1,3-cyclohexanedione derivative, benzhydrazide derivative, gallic acid derivative, pyrazol-3-one derivative, or a tropone derivative.

The present invention provides novel activators of HDAC1.

In certain embodiments, the HDAC1 activator is a chelating agent. In certain embodiments, the HDAC1 activator is a desferrioxamine derivative. In certain embodiments, the chelating agent is of the formula:

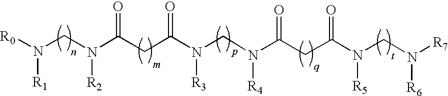

wherein
n is an integer between 1 and 6, inclusive;
m is an integer between 1 and 6, inclusive;
p is an integer between 1 and 6, inclusive;
q is an integer between 1 and 6, inclusive;
t is an integer between 1 and 6, inclusive;
R₀ is hydrogen, hydroxyl, acyl, or a nitrogen protecting group;
R₁ is hydrogen, hydroxyl, acyl, or a nitrogen protecting group;
R₂ is hydrogen, hydroxyl, acyl, or a nitrogen protecting group;
R₃ is hydrogen, hydroxyl, acyl, or a nitrogen protecting group;
R₄ is hydrogen, hydroxyl, acyl, or a nitrogen protecting group;
R₅ is hydrogen, hydroxyl, acyl, or a nitrogen protecting group;

R₆ is hydrogen, hydroxyl, acyl, or a nitrogen protecting group;

R₇ is hydrogen, hydroxyl, acyl, or a nitrogen protecting group; and a pharmaceutically acceptable salt thereof.

In certain embodiments, n is 1. In certain embodiments, n is 2. In certain embodiments, n is 3. In certain embodiments, n is 4. In certain embodiments, n is 4. In certain embodiments, n is 5. In certain embodiments, n is 6.

In certain embodiments, m is 1. In certain embodiments, m is 2. In certain embodiments, m is 3. In certain embodiments, m is 4. In certain embodiments, m is 4. In certain embodiments, m is 5. In certain embodiments, m is 6.

In certain embodiments, p is 1. In certain embodiments, p is 2. In certain embodiments, p is 3. In certain embodiments, p is 4. In certain embodiments, p is 4. In certain embodiments, p is 5. In certain embodiments, p is 6.

In certain embodiments, q is 1. In certain embodiments, q is 2. In certain embodiments, q is 3. In certain embodiments, q is 4. In certain embodiments, q is 4. In certain embodiments, q is 5. In certain embodiments, q is 6.

In certain embodiments, t is 1. In certain embodiments, t is 2. In certain embodiments, t is 3. In certain embodiments, t is 4. In certain embodiments, t is 4. In certain embodiments, t is 5. In certain embodiments, t is 6.

In certain embodiments, R₀ is hydrogen. In certain embodiments, R₀ is —OH. In certain embodiments, R₀ is alkoxy. In certain embodiments, R₀ is acyl. In certain embodiments, R₀ is acetyl. In certain embodiments, R₀ is C₁-C₆ alkyl. In certain embodiments, R₀ is a nitrogen protecting group. In certain embodiments, R₀ is a nitrogen protecting group, wherein the nitrogen protecting group is selected from the group consisting of benzyl, p-methoxybenzyl, allyl, trityl, methyl, acetyl, trichloroacetamide, trifluoroacetamide, pent-4-enamide, phthalimide, chlorinated phthalimide, methyl carbamate, t-butyl carbamate, benzyl carbamate, allyl carbamate, 2-(trimethylsilyl)ethyl carbamate, 2,2,2-trichloroethyl carbamate, 9-fluorenylmethyl carbamate, tosyl, and sulfonamides.

In certain embodiments, R₁ is hydrogen. In certain embodiments, R₁ is —OH. In certain embodiments, R₁ is alkoxy. In certain embodiments, R₁ is acyl. In certain embodiments, R₁ is acetyl. In certain embodiments, R₁ is C₁-C₆ alkyl. In certain embodiments, R₁ is a nitrogen protecting group. In certain embodiments, R₁ is a nitrogen protecting group, wherein the nitrogen protecting group is selected from the group consisting of benzyl, p-methoxybenzyl, allyl, trityl, methyl, acetyl, trichloroacetamide, trifluoroacetamide, pent-4-enamide, phthalimide, chlorinated phthalimide, methyl carbamate, t-butyl carbamate, benzyl carbamate, allyl carbamate, 2-(trimethylsilyl)ethyl carbamate, 2,2,2-trichloroethyl carbamate, 9-fluorenylmethyl carbamate, tosyl, and sulfonamides.

In certain embodiments, R₂ is hydrogen. In certain embodiments, R₂ is —OH. In certain embodiments, R₂ is alkoxy. In certain embodiments, R₂ is acyl. In certain embodiments, R₂ is acetyl. In certain embodiments, R₂ is C₁-C₆ alkyl. In certain embodiments, R₂ is a nitrogen protecting group. In certain embodiments, R₂ is a nitrogen to protecting group, wherein the nitrogen protecting group is selected from the group consisting of benzyl, p-methoxybenzyl, allyl, trityl, methyl, acetyl, trichloroacetamide, trifluoroacetamide, pent-4-enamide, phthalimide, chlorinated phthalimide, methyl carbamate, t-butyl carbamate, benzyl carbamate, allyl carbamate, 2-(trimethylsilyl)ethyl carbamate, 2,2,2-trichloroethyl carbamate, 9-fluorenylmethyl carbamate, tosyl, and sulfonamides.

In certain embodiments, R₃ is hydrogen. In certain embodiments, R₃ is —OH. In certain embodiments, R₃ is alkoxy. In certain embodiments, R₃ is acyl. In certain embodiments, R₃ is acetyl. In certain embodiments, R₃ is C₁-C₆ alkyl. In certain embodiments, R₃ is a nitrogen protecting group. In certain embodiments, R₃ is a nitrogen protecting group, wherein the nitrogen protecting group is selected from the group consisting of benzyl, p-methoxybenzyl, allyl, trityl, methyl, acetyl, trichloroacetamide, trifluoroacetamide, pent-4-enamide, phthalimide, chlorinated phthalimide, methyl carbamate, t-butyl carbamate, benzyl carbamate, allyl carbamate, 2-(trimethylsilyl)ethyl carbamate, 2,2,2-trichloroethyl carbamate, 9-fluorenylmethyl carbamate, tosyl, and sulfonamides.

In certain embodiments, R₄ is hydrogen. In certain embodiments, R₄ is —OH. In certain embodiments, R₄ is alkoxy. In certain embodiments, R₄ is acyl. In certain embodiments, R₄ is acetyl. In certain embodiments, R₄ is C₁-C₆ alkyl. In certain embodiments, R₄ is a nitrogen protecting group. In certain embodiments, R₄ is a nitrogen protecting group, wherein the nitrogen protecting group is selected from the group consisting of benzyl, p-methoxybenzyl, allyl, trityl, methyl, acetyl, trichloroacetamide, trifluoroacetamide, pent-4-enamide, phthalimide, chlorinated phthalimide, methyl carbamate, t-butyl carbamate, benzyl carbamate, allyl carbamate, 2-(trimethylsilyl)ethyl carbamate, 2,2,2-trichloroethyl carbamate, 9-fluorenylmethyl carbamate, tosyl, and sulfonamides.

In certain embodiments, R₅ is hydrogen. In certain embodiments, R₅ is —OH. In certain embodiments, R₅ is alkoxy. In certain embodiments, R₅ is acyl. In certain embodiments, R₅ is acetyl. In certain embodiments, R₅ is C₁-C₆ alkyl. In certain embodiments, R₅ is a nitrogen protecting group. In certain embodiments, R₅ is a nitrogen protecting group, wherein the nitrogen protecting group is selected from the group consisting of benzyl, p-methoxybenzyl, allyl, trityl, methyl, acetyl, trichloroacetamide, trifluoroacetamide, pent-4-enamide, phthalimide, chlorinated phthalimide, methyl carbamate, t-butyl carbamate, benzyl carbamate, allyl carbamate, 2-(trimethylsilyl)ethyl carbamate, 2,2,2-trichloroethyl carbamate, 9-fluorenylmethyl carbamate, tosyl, and sulfonamides.

In certain embodiments, R₆ is hydrogen. In certain embodiments, R₆ is —OH. In certain embodiments, R₆ is alkoxy. In certain embodiments, R₆ is acyl. In certain embodiments, R₆ is acetyl. In certain embodiments, R₆ is C₁-C₆ alkyl. In certain embodiments, R₆ is a nitrogen protecting group. In certain embodiments, R₆ is a nitrogen protecting group, wherein the nitrogen protecting group is selected from the group consisting of benzyl, p-methoxybenzyl, allyl, trityl, methyl, acetyl, trichloroacetamide, trifluoroacetamide, pent-4-enamide, phthalimide, chlorinated phthalimide, methyl carbamate, t-butyl carbamate, benzyl carbamate, allyl carbamate, 2-(trimethylsilyl)ethyl carbamate, 2,2,2-trichloroethyl carbamate, 9-fluorenylmethyl carbamate, tosyl, and sulfonamides.

In certain embodiments, R₇ is hydrogen. In certain embodiments, R₇ is —OH. In certain embodiments, R₇ is alkoxy. In certain embodiments, R₇ is acyl. In certain embodiments, R₇ is acetyl. In certain embodiments, R₇ is C₁-C₆ alkyl. In certain embodiments, R₇ is a nitrogen protecting group. In certain embodiments, R₇ is a nitrogen protecting group, wherein the nitrogen protecting group is selected from the group consisting of benzyl, p-methoxybenzyl, allyl, trityl, methyl, acetyl, trichloroacetamide, trifluoroacetamide, pent-4-enamide, phthalimide, chlorinated phthalimide, methyl carbamate, t-butyl carbamate, benzyl carbamate, allyl carbamate, 2-(trimethylsilyl)ethyl carbamate, 2,2,2-trichloroethyl carbamate, 9-fluorenylmethyl carbamate, tosyl, and sulfonamides. In certain embodiments, the HDAC1 activator is desferrioxamine.

In certain embodiments, the HDAC1 activator is a catechol-containing compound. In certain embodiments, the catechol-containing compound is of the formula:

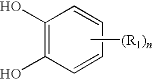

wherein
n is an integer between 1 and 4, inclusive;
each of R₁ is independently hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{A}; —C(═O)R_{A}; —CO₂R_{A}; —CN; —SCN; —SR_{A}; —SOR_{A}; —SO₂R_{A}; —NO₂; —N₃; —N(R_{A})₂; —NHC(═O)R_{A}; —NR_{A}C(═O)N(R_{A})₂; —OC(═O)OR_{A}; —OC(═O)R_{A}; —OC(═O)N(R_{A})₂; —NR_{A}C(═O)OR_{A}; or —C(R_{A})₃; wherein each occurrence of R_{A} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety; and pharmaceutically acceptable salts thereof.

In certain embodiments, n is 1. In certain embodiments, n is 2. In certain embodiments, n is 3. In certain embodiments, n is 4. In certain embodiments where n is at least 2, two R₁ moieties are taken together to form a cyclic structure.

In certain embodiments, R₁ is halogen. In certain embodiments, R₁ is cyclic or acyclic, substituted or unsubstituted, branched or unbranched aliphatic. In certain embodiments, R₁ is cyclic or acyclic, substituted or unsubstituted, branched or unbranched heteroaliphatic. In certain embodiments, R₁ is acyclic, branched or unbranched, substituted or unsubstituted aliphatic. In certain embodiments, R₁ is acyclic, branched or unbranched, substituted or unsubstituted alkyl. In certain embodiments, R₁ is acyclic, branched or unbranched, substituted or unsubstituted C₁-C₆ alkyl. In certain embodiments, R₁ is acyclic, branched or unbranched substituted C₁-C₆ alkyl. In certain embodiments, R₁ is substituted with an amino group. In certain embodiments, R₁ is substituted with an alkylamino group. In certain embodiments, R₁ is substituted with a dialkylamino group. In certain embodiments, R₁ is substituted with a hydroxyl group. In certain embodiments, R₁ is substituted with a alkyoxy group. In certain embodiments, R₁ is substituted with an acyl group. In certain embodiments, R₁ is substituted with a carboxylic acid group. In certain embodiments, R₁ is substituted with an aryl moiety. In certain embodiments, R₁ is substituted with a phenyl moiety. In certain embodiments, R₁ is substituted with a heteroaryl moiety. In certain embodiments, R₁ is acyclic, branched or unbranched, substituted or unsubstituted alkenyl. In certain embodiments, R₁ is acyclic, branched or unbranched, substituted or unsubstituted alkynyl. In certain embodiments, R₁ is substituted or unsubstituted, branched or unbranched acyl. In certain embodiments, R₁ is substituted or unsubstituted, branched or unbranched aryl. In certain embodiments, R₁ is substituted or unsubstituted, branched or unbranched heteroaryl.

In certain embodiments, the compound is of one the formulae:

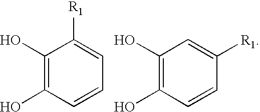

In certain embodiments, the compound is of one of the formulae:

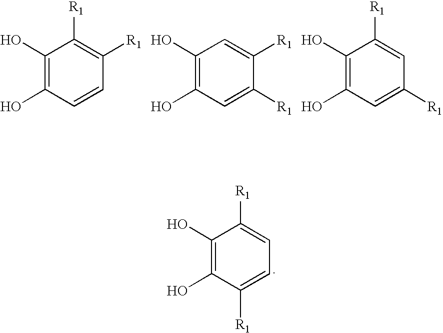

In certain embodiments, the compound is of one the formulae:

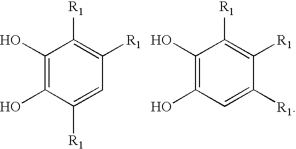

In certain embodiments, the compound is of the formula:

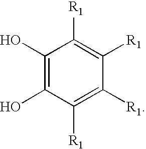

In certain embodiments, the compound is of the formula:

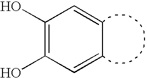

wherein

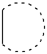

is a substituted or unsubstituted, aromatic or nonaromatic, carbocyclic or heterocyclic moiety. In certain embodiments,

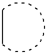

is carbocyclic. In certain embodiments,

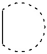

is heterocyclic. In certain embodiments,

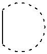

is substituted. In certain embodiments,

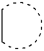

is substituted. In certain embodiments,

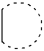

is five-membered, six-membered, or seven-membered. In certain embodiments,

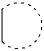

is a seven-membered heterocylic moiety. In certain embodiments,

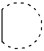

is a seven-membered heterocylic moiety with one nitrogen atom.

In certain embodiments, the compound is levonordefrin, methyldopa, or R(+)-SKF-81297.

In certain embodiments, the HDAC1 activator is a phosphorus-containing compound. In certain embodiments, the HDAC1 activator is a phosphate-containing compound. In certain embodiments, the HDAC1 activator is a phosphonate-containing compound. In certain embodiments, the HDAC1 activator is of the formula:

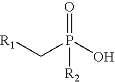

wherein
R₁ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{A}; —C(═O)R_{A}; —CO₂R_{A}; —CN; —SCN; —SR_{A}; —SOR_{A}; —SO₂R_{A}; —NO₂; —N₃; —N(R_{A})₂; —NHC(═O)R_{A}; —NR_{A}C(═O)N(R_{A})₂; —OC(═O)OR_{A}; —OC(═O)R_{A}; —OC(═O)N(R_{A})₂; —NR_{A}C(═O)OR_{A}; or —C(R_{A})₃; wherein each occurrence of R_{A} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

R₂ is cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{B}; —OH; or —C(R_{B})₃; wherein each occurrence of R_{B} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety; and pharmaceutically acceptable salts thereof.

In certain embodiments, R₁ is cyclic or acyclic, substituted or unsubstituted, branched or unbranched aliphatic. In certain embodiments, R₁ is cyclic or acyclic, substituted or unsubstituted, branched or unbranched heteroaliphatic. In certain embodiments, R₁ is acyclic, branched or unbranched, substituted or unsubstituted aliphatic. In certain embodiments, R₁ is to acyclic, branched or unbranched, substituted or unsubstituted alkyl. In certain embodiments, R₁ is acyclic, branched or unbranched, substituted or unsubstituted C₁-C₆ alkyl. In certain embodiments, R₁ is a substituted or unsubstituted carbocyclic moiety. In certain embodiments, R₁ is a substituted or unsubstituted heterocyclic moiety. In certain embodiments, R₁ is substituted heterocyclic. In certain embodiments, R₁ is unsubstituted piperidinyl. In certain embodiments, R₁ is substituted piperidinyl. In certain embodiments, R₁ is a substituted or unsubstituted, monocyclic heterocyclic moiety. In certain embodiments, R₁ is a substituted or unsubstituted bicyclic moiety. In certain embodiments, R₁ is acyclic, branched or unbranched substituted C₁-C₆ alkyl. In certain embodiments, R₁ is hydroxyalkyl. In certain embodiments, R₁ is hydroxymethyl. In certain embodiments, R₁ is hydroxyethyl. In certain embodiments, R₁ is hydroxypropyl. In certain embodiments, R₁ is aminoalkyl. In certain embodiments, R₁ is aminomethyl. In certain embodiments, R₁ is aminoethyl. In certain embodiments, R₁ is aminopropyl. In certain embodiments, R₁ is acyclic, branched or unbranched, substituted or unsubstituted alkenyl. In certain embodiments, R₁ is acyclic, branched or unbranched, substituted or unsubstituted alkynyl. In certain embodiments, R₁ is substituted or unsubstituted heterocylic. In certain embodiments, R₁ is substituted or unsubstituted, branched or unbranched acyl. In certain embodiments, R₁ is substituted or unsubstituted, branched or unbranched aryl. In certain embodiments, R₁ is substituted or unsubstituted, branched or unbranched heteroaryl. In certain embodiments, R₁ is substituted with an amino group. In certain embodiments, R₁ is substituted with an alkylamino group. In certain embodiments, R₁ is substituted with a dialkylamino group. In certain embodiments, R₁ is substituted with a hydroxyl group. In certain embodiments, R₁ is substituted with an alkoxy group. In certain embodiments, R₁ is substituted with an acyl group. In certain embodiments, R₁ is substituted with a carboxylic acid group. In certain embodiments, R₁ is substituted with a phosphate moiety. In certain embodiments, R₁ is substituted with an aryl moiety. In certain embodiments, R₁ is substituted with a phenyl moiety. In certain embodiments, R₁ is substituted with a heteroaryl moiety.

In certain embodiments, R₂ is C₁-C₆ alkyl. In certain embodiments, R₂ is methyl. In certain embodiments, R₂ is ethyl. In certain embodiments, R₂ is propyl. In certain embodiments, R₂ is butyl. In certain embodiments, R₂ is —OH. In certain embodiments, R₂ is —OR_{B}.

In certain embodiments, the compound is of the formula:

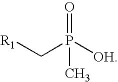

In certain embodiments, the compound is of the formula:

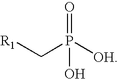

In certain embodiments, the compound is LY 235959, CGS 19755, SK&F97541, or etidronic acid.

In certain embodiments, the HDAC1 activator is of the formula:

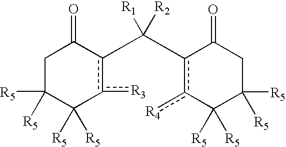

wherein
each [symbol] is independently a single or double bond;
each of R₁ and R₂ is independently hydrogen; cyclic or acyclic, branched or unbranched, substituted or unsubstituted aliphatic; cyclic or acyclic, substituted or unsubstituted, branched or unbranched heteroaliphatic; substituted or unsubstituted, branched or unbranched acyl; substituted or unsubstituted aryl, substituted or unsubstituted, branched or unbranched heteroaryl; —OR_{A}; —C(═O)R_{A}; —CO₂R_{A}; —CN; —SCN; —SR_{A}; —SOR_{A}; —SO₂R_{A}; —NO₂; —N₃; —N(R_{A})₂; —NHC(═O)R_{A}; —NR_{A}C(═O)N(R_{A})₂; —OC(═O)OR_{A}; —OC(═O)R_{A}; —OC(═O)N(R_{A})₂; —NR_{A}C(═O)OR_{A}; or —C(R_{A})₃; wherein each occurrence of R_{A} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety; and pharmaceutically acceptable salts thereof.

each of R₃, and R₄ is independently —OH, alkoxy, —Oacyl, ═O, or wherein R₃ and R₄ are taken together to form a cyclic structure;

each of R₅ is independently hydrogen; cyclic or acyclic, branched or unbranched, substituted or unsubstituted aliphatic; and pharmaceutically acceptable salts thereof.

In certain embodiments, R₁ is hydrogen. In certain embodiments, R₁ is cyclic or acyclic, branched or unbranched, substituted or unsubstituted aliphatic. In certain embodiments, R₁ is acyclic, branched or unbranched, substituted or unsubstituted alkyl. In certain embodiments, R₁ is acyclic, branched or unbranched, substituted or unsubstituted C₁-C₆ alkyl. In certain embodiments, R₁ is acyclic, branched or unbranched substituted C₁-C₆ alkyl. In certain embodiments, R₁ is acyclic, branched or unbranched, substituted or unsubstituted C₂-C₆ alkenyl. In certain embodiments, R₁ is acyclic, branched or unbranched, substituted or unsubstituted C₂-C₆ alkynyl. In certain embodiments, R₁ is substituted or unsubstituted aryl. In certain embodiments, R₁ is substituted or unsubstituted heteroaryl. In certain embodiments, R₁ is

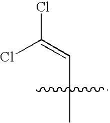

In certain embodiments, R₁ is

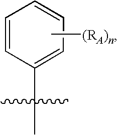

wherein n is an integer between 0 and 5, inclusive, and wherein each occurrence of R_{A} is independently a hydrogen, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety. In certain embodiments, R₁ is phenyl. In certain embodiments, R₁ is substituted or unsubstituted benzyl. In certain embodiments, R₁ is

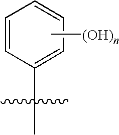

wherein n is an integer between 0 and 5. In certain embodiments, R₁ is

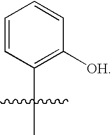

In certain embodiments, R₂ is hydrogen. In certain embodiments, R₂ is cyclic or acyclic, branched or unbranched, substituted or unsubstituted aliphatic. In certain embodiments, R₂ is acyclic, branched or unbranched, substituted or unsubstituted alkyl. In certain embodiments, R₂ is acyclic, branched or unbranched, substituted or unsubstituted C₁-C₆ alkyl. In certain embodiments, R₂ is acyclic, branched or unbranched substituted C₁-C₆ alkyl. In certain embodiments, R₂ is acyclic, branched or unbranched, substituted or unsubstituted C₂-C₆ alkenyl. In certain embodiments, R₂ is acyclic, branched or unbranched, substituted or unsubstituted C₂-C₆ alkynyl. In certain embodiments, R₂ is substituted or unsubstituted aryl. In certain embodiments, R₂ is substituted or unsubstituted heteroaryl. In certain embodiments, R₂ is

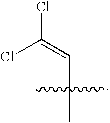

In certain embodiments, R₂ is

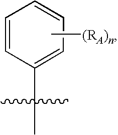

wherein n is an integer between 0 and 5, inclusive, and wherein each occurrence of R_{A} is independently a hydrogen, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety. In certain embodiments, R₂ is phenyl. In certain embodiments, R₂ is substituted or unsubstituted benzyl. In certain embodiments, R₂ is

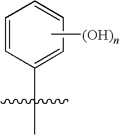

wherein n is an integer between 0 and 5. In certain embodiments, R₂ is

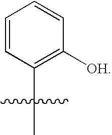

In certain embodiments, both R₁ and R₂ are hydrogen. In certain embodiments, at least one of R₁ and R₂ is hydrogen.

In certain embodiments, R₃ is —OH. In certain embodiments, R₃ is alkoxy. In certain embodiments, R₃ is —Oacyl. In certain embodiments, R₃ is ═O.

In certain embodiments, R₄ is —OH. In certain embodiments, R₄ is alkoxy. In certain embodiments, R₄ is —Oacyl. In certain embodiments, R₄ is ═O.

In certain embodiments, R₃ and R₄ are taken together to form the cyclic structure

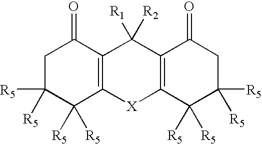

wherein X is selected from the group consisting of CH₂, NH, C═O, P, and S. In certain embodiments, R₃ and R₄ are taken together via an —O— linkage to form the cyclic structure

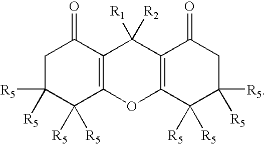

In certain embodiments, R₅ is hydrogen. In certain embodiments, R₅ is cyclic or acyclic, branched or unbranched, substituted or unsubstituted aliphatic. In certain embodiments, R₅ is acyclic, branched or unbranched substituted C₁-C₆ alkyl. In certain embodiments, R₅ is methyl. In certain embodiments, R₅ substituents bound to the same carbon are geminal di-methyl.

to In certain embodiments, the HDAC1 activator is

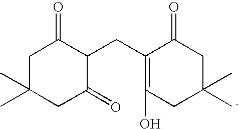

In certain embodiments, the HDAC1 activator is

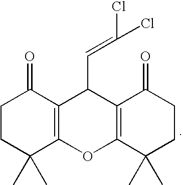

In certain embodiments, the HDAC1 activator is

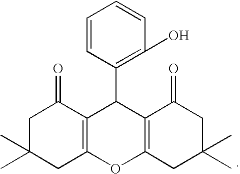

In certain embodiments, the HDAC1 activator is a flavonoid or a derivative thereof.

In certain embodiments, the HDAC1 activator is of the formula:

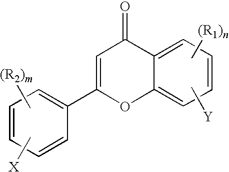

wherein
n is an integer between 0 and 4, inclusive;
m is an integer between 0 and 5, inclusive;

each of R₁ and R₂ is independently —OH; alkoxy; —Oacyl; —OAc; —OP_{G}; substituted or unsubstituted aryl;

wherein either R₁ or R₂ can be a second HDAC1 activator moiety; and pharmaceutically acceptable salts thereof.

In certain embodiments, n is 0. In certain embodiments, n is 1. In certain to embodiments, n is 2. In certain embodiments, n is 3. In certain embodiments, n is 4.

In certain embodiments, m is 0. In certain embodiments, m is 1. In certain embodiments, m is 2. In certain embodiments, m is 3. In certain embodiments, m is 4. In certain embodiments, m is 5.

In certain embodiments, R₁ is —OH. In certain embodiments, R₁ is alkoxy. In certain embodiments, R₁ is C₁-C₆ alkoxy. In certain embodiments, R₁ is methoxy. In certain embodiments, R₁ is —Oacyl. In certain embodiments, R₁ is —OAc. In certain embodiments, R₁ is —OP_{G}. In certain embodiments, R₁ is substituted or unsubstituted aryl. In certain embodiments, R₁ is substituted or unsubstituted phenyl.

In certain embodiments, R₂ is —OH. In certain embodiments, R₂ is alkoxy. In certain embodiments, R₂ is C₁-C₆ alkoxy. In certain embodiments, R₂ is methoxy. In certain embodiments, R₂ is —Oacyl. In certain embodiments, R₂ is —OAc. In certain embodiments, R₂ is —OP_{G}. In certain embodiments, R₂ is substituted or unsubstituted aryl. In certain embodiments, R₂ is substituted or unsubstituted phenyl.

In certain embodiments, the HDAC1 activator is of the formula:

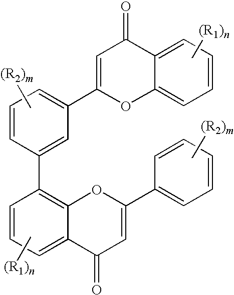

wherein
n is an integer between 0 and 4, inclusive;
m is an integer between 0 and 4, inclusive;

each of R₁ and R₂ is independently —OH; alkoxy; —Oacyl; —OAc; —OP_{G}; substituted or unsubstituted aryl; and pharmaceutically acceptable salts thereof.

In certain embodiments, n is 0. In certain embodiments, n is 1. In certain embodiments, n is 2. In certain embodiments, n is 3. In certain embodiments, n is 4.

In certain embodiments, m is 0. In certain embodiments, m is 1. In certain to embodiments, m is 2. In certain embodiments, m is 3. In certain embodiments, m is 4.

In certain embodiments, R₁ is —OH. In certain embodiments, R₁ is alkoxy. In certain embodiments, R₁ is C₁-C₆ alkoxy. In certain embodiments, R₁ is methoxy. In certain embodiments, R₁ is —Oacyl. In certain embodiments, R₁ is —OAc. In certain embodiments, R₁ is —OP_{G}. In certain embodiments, R₁ is substituted or unsubstituted aryl. In certain embodiments, R₁ is substituted or unsubstituted phenyl.

In certain embodiments, R₂ is —OH. In certain embodiments, R₂ is alkoxy. In certain embodiments, R₂ is C₁-C₆ alkoxy. In certain embodiments, R₂ is methoxy. In certain embodiments, R₂ is —Oacyl. In certain embodiments, R₂ is —OAc. In certain embodiments, R₂ is —OP_{G}. In certain embodiments, R₂ is substituted or unsubstituted aryl. In certain embodiments, R₂ is substituted or unsubstituted phenyl. In certain embodiments, the HDAC1 activator is

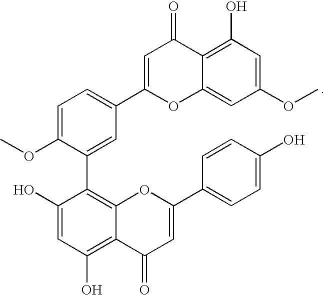

In certain embodiments, the HDAC1 activator is

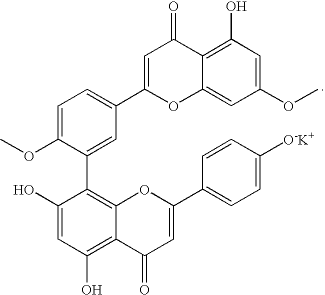

In certain embodiments, the HDAC1 activator is

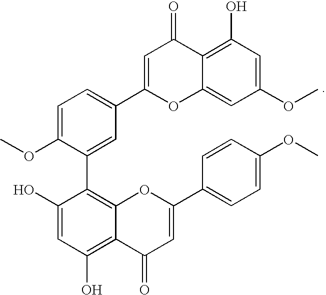

In certain embodiments, the HDAC1 activator is gambogic acid or a derivative thereof. In certain embodiments, the HDAC1 activator is of the formula:

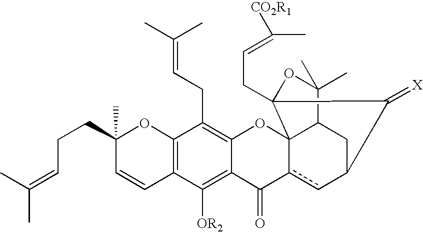

wherein

[symbol] is independently a single or double bond;

R₁ is hydrogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched aryl; substituted or unsubstituted, branched or unbranched heteroaryl;

R₂ is hydrogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —C(═O)R_{B}; —CO₂R_{B}; or —C(R_{B})₃; wherein each occurrence of R_{B} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

X is ═O,

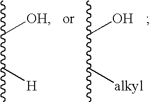

and pharmaceutically acceptable salts thereof.

In certain embodiments, [symbol] is a single bond. In certain embodiments, [symbol] is a double bond.

In certain embodiments, R₁ is hydrogen. In certain embodiments, R₂ is acyclic, branched or unbranched, substituted or unsubstituted alkyl. In certain embodiments, R₂ is acyclic, branched or unbranched, substituted or unsubstituted C₁-C₆ alkyl. In certain embodiments, R₂ is acyclic, branched or unbranched substituted C₁-C₆ alkyl. In certain embodiments, R₂ is acyclic, branched or unbranched, substituted or unsubstituted C₂-C₆ alkenyl. In certain embodiments, R₂ is acyclic, branched or unbranched, substituted or unsubstituted C₂-C₆ alkynyl. In certain embodiments, R₁ is methyl. In certain embodiments, R₁ is ethyl. In certain embodiments, R₁ is propyl. In certain embodiments, R₁ is butyl.

In certain embodiments, R₂ is hydrogen. In certain embodiments, R₂ is substituted or unsubstituted, branched or unbranched alkyl. In certain embodiments, R₂ is C₁-C₆ alkyl. In certain embodiments, R₂ is methyl. In certain embodiments, R₂ is ethyl. In certain embodiments, R₂ is propyl. In certain embodiments, R₂ is butyl. In certain embodiments, R₂ is —Oacyl. In certain embodiments, R₂ is —OAc. In certain embodiments, R₂ is —OP_{G}.

In certain embodiments, X is ═O. In certain embodiments, X is

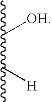

In certain embodiments, the HDAC1 activator is

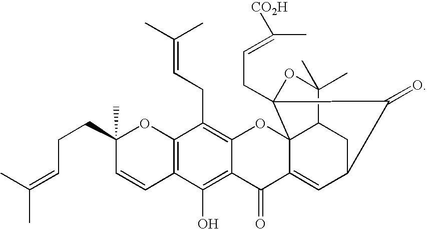

In certain embodiments, the HDAC1 activator is

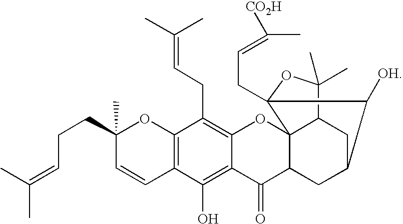

In certain embodiments, the HDAC1 activator is of the formula:

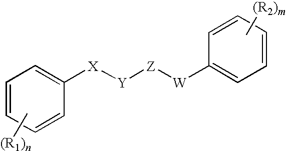

wherein

n is an integer between 0 and 5, inclusive;

to m is an integer between 0 and 5, inclusive;

each X, Y, and Z is independently selected from the list consisting of CH₂, NH, C═O, and O; and

wherein W is either absent or selected from the list consisting of CH₂, NH, C═O, and O;

each of R₁ and R₂ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{A}; —C(═O)R_{A}; —CO₂R_{A}; —CN; —SCN; —SR_{A}; —SOR_{A}; —SO₂R_{A}; —NO₂; —N₃; —N(R_{A})₂; —NHC(═O)R_{A}; —NR_{A}C(═O)N(R_{A})₂; —OC(═O)OR_{A}; —OC(═O)R_{A}; —OC(═O)N(R_{A})₂; —NR_{A}C(═O)OR_{A}; or —C(R_{A})₃; wherein each occurrence of R_{A} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety; and pharmaceutically acceptable salts thereof.

In certain embodiments, n is 0. In certain embodiments, n is 1. In certain embodiments, n is 2. In certain embodiments, n is 3. In certain embodiments, n is 4. In certain embodiments, n is 5.

In certain embodiments, m is 0. In certain embodiments, m is 1. In certain embodiments, m is 2. In certain embodiments, m is 3. In certain embodiments, m is 4. In certain embodiments, m is 5.

In certain embodiments, X is CH₂. In certain embodiments, X is NH. In certain embodiments, X is C═O. In certain embodiments, X is O.

In certain embodiments, Y is CH₂. In certain embodiments, Y is NH. In certain embodiments, Y is C═O. In certain embodiments, Y is O.

In certain embodiments, Z is CH₂. In certain embodiments, Z is NH. In certain embodiments, Z is C═O. In certain embodiments, Z is O.

In certain embodiments, W is absent. In certain embodiments, W is CH₂. In certain embodiments, W is NH. In certain embodiments, W is C═O. In certain embodiments, W is O.

In certain embodiments, R₁ is hydrogen. In certain embodiments, R₁ is halogen. In certain embodiments, R₁ is chloro. In certain embodiments, R₁ is cyclic or acyclic, substituted or unsubstituted, branched or unbranched aliphatic. In certain embodiments, R₁ is acyclic, branched or unbranched, substituted or unsubstituted alkyl. In certain embodiments, R₁ is acyclic, branched or unbranched, substituted or unsubstituted C₁-C₆ alkyl. In certain embodiments, R₁ is acyclic, branched or unbranched substituted C₁-C₆ alkyl. In certain embodiments, R₁ is acyclic, branched or unbranched, substituted or unsubstituted C₂-C₆ alkenyl. In certain embodiments, R₁ is acyclic, branched or unbranched, substituted or unsubstituted C₂-C₆ alkynyl. In certain embodiments, R₁ is methyl. In certain embodiments, R₁ is ethyl. In certain embodiments, R₁ is propyl. In certain embodiments, R₁ is butyl. In certain embodiments, R₁ is F. In certain embodiments, R₁ is -CN. In certain embodiments, R₁ is —NO₂. In certain embodiments, R₁ is -OR_{A}. In certain embodiments, R₁ is —OC(═O)R_{A}.

In certain embodiments, R₁ is —OC(═O)R_{A}, wherein R_{A} is aryl. In certain embodiments, R₁ is —OC(═O)R_{A}, wherein R_{A} is 4-nitrophenyl.

In certain embodiments, R₂ is hydrogen. In certain embodiments, R₂ is halogen. In certain embodiments, R₂ is chloro. In certain embodiments, R₂ is cyclic or acyclic, substituted or unsubstituted, branched or unbranched aliphatic. In certain embodiments, R₂ is acyclic, branched or unbranched, substituted or unsubstituted alkyl. In certain embodiments, R₂ is acyclic, branched or unbranched, substituted or unsubstituted C₁-C₆ alkyl. In certain embodiments, R₂ is acyclic, branched or unbranched substituted C₁-C₆ alkyl. In certain embodiments, R₂ is acyclic, branched or unbranched, substituted or unsubstituted C₂-C₆ alkenyl. In certain embodiments, R₂ is acyclic, branched or unbranched, substituted or unsubstituted C₂-C₆ alkynyl. In certain embodiments, R₂ is methyl. In certain embodiments, R₂ is ethyl. In certain embodiments, R₂ is propyl. In certain embodiments, R₂ is butyl. In certain embodiments, R₂ is F. In certain embodiments, R₂ is —CN. In certain embodiments, R₂ is —NO₂. In certain embodiments, R₂ is —OR_{A}. In certain embodiments, R₂ is —OC(═O)R_{A}. In certain embodiments, R₂ is —OC(═O)R_{A}, wherein R_{A} is aryl. In certain embodiments, R₂ is —OC(═O)R_{A}, wherein R_{A} is 4-nitrophenyl.

In some embodiments

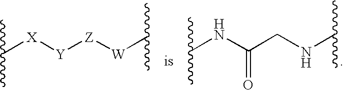

In some embodiments

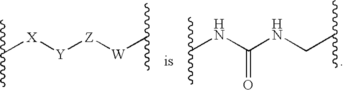

In some embodiments

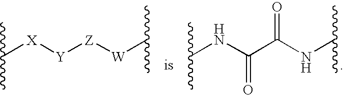

In some embodiments

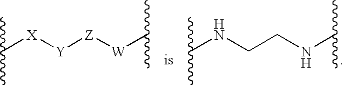

In some embodiments

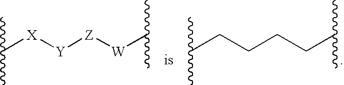

In certain embodiments, the HDAC1 activator is

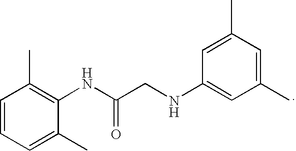

In certain embodiments, the HDAC1 activator is of the formula:

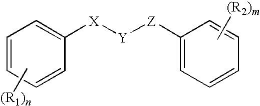

wherein

n is an integer between 0 and 5, inclusive;

m is an integer between 0 and 5, inclusive;

each X, Y, and Z is independently selected from the list consisting of CH₂, NH, C═O, O, and S;

each of R₁ and R₂ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; to substituted or unsubstituted, branched or unbranched aryl; substituted or unsubstituted, branched or unbranched heteroaryl; —OR_{A}; —C(═O)R_{A}; —CO₂R_{A}; —CN; —SCN; —SR_{A}; —SOR_{A}; —SO₂R_{A}; —NO₂; —N₃; —N(R_{A})₂; —NHC(═O)R_{A}; —NR_{A}C(═O)N(R_{A})₂; —OC(═O)OR_{A}; —OC(═O)R_{A}; —OC(═O)N(R_{A})₂; —NR_{A}C(═O)OR_{A}; or —C(R_{A})₃; wherein each occurrence of R_{A} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety; and pharmaceutically acceptable salts thereof.

In certain embodiments, n is 0. In certain embodiments, n is 1. In certain embodiments, n is 2. In certain embodiments, n is 3. In certain embodiments, n is 4. In certain embodiments, n is 5.

In certain embodiments, m is 0. In certain embodiments, m is 1. In certain embodiments, m is 2. In certain embodiments, m is 3. In certain embodiments, m is 4. In certain embodiments, m is 5.

In certain embodiments, X is CH₂. In certain embodiments, X is NH. In certain embodiments, X is C═O. In certain embodiments, X is O. In certain embodiments, X is S.

In certain embodiments, Y is CH₂. In certain embodiments, Y is NH. In certain embodiments, Y is C═O. In certain embodiments, Y is O. In certain embodiments, Y is S.

In certain embodiments, Z is CH₂. In certain embodiments, Z is NH. In certain embodiments, Z is C═O. In certain embodiments, Z is O. In certain embodiments, Z is S.

In certain embodiments, R₁ is hydrogen. In certain embodiments, R₁ is halogen. In certain embodiments, R₁ is chloro. In certain embodiments, R₁ is cyclic or acyclic, substituted or unsubstituted, branched or unbranched aliphatic. In certain embodiments, R₁ is acyclic, branched or unbranched, substituted or unsubstituted alkyl. In certain embodiments, R₁ is acyclic, branched or unbranched, substituted or unsubstituted C₁-C₆ alkyl. In certain embodiments, R₁ is acyclic, branched or unbranched substituted C₁-C₆ alkyl. In certain embodiments, R₁ is acyclic, branched or unbranched, substituted or unsubstituted C₂-C₆ alkenyl. In certain embodiments, R₁ is acyclic, branched or unbranched, substituted or unsubstituted C₂-C₆ alkynyl. In certain embodiments, R₁ is methyl. In certain embodiments, R₁ is ethyl. In certain embodiments, R₁ is propyl. In certain embodiments, R₁ is butyl. In certain embodiments, R₁ is F. In certain embodiments, R₁ is -CN. In certain embodiments, R₁ is —NO₂. In certain embodiments, R₁ is —OR_{A}. In certain embodiments, R₁ is —OC(═O)R_{A}. In certain embodiments, R₁ is —OC(═O)R_{A}, wherein R_{A} is aryl. In certain embodiments, R₁ is —OC(═O)R_{A}, wherein R_{A} is 4-nitrophenyl.

In certain embodiments, R₂ is hydrogen. In certain embodiments, R₂ is halogen. In certain embodiments, R₂ is chloro. In certain embodiments, R₂ is cyclic or acyclic, substituted or unsubstituted, branched or unbranched aliphatic. In certain embodiments, R₂ is acyclic, branched or unbranched, substituted or unsubstituted alkyl. In certain embodiments, R₂ is acyclic, branched or unbranched, substituted or unsubstituted C₁-C₆ alkyl. In certain embodiments, R₂ is acyclic, branched or unbranched substituted C₁-C₆ alkyl. In certain embodiments, R₂ is acyclic, branched or unbranched, substituted or unsubstituted C₂-C₆ alkenyl. In certain embodiments, R₂ is acyclic, branched or unbranched, substituted or unsubstituted C₂-C₆ alkynyl. In certain embodiments, R₂ is methyl. In certain embodiments, R₂ is ethyl. In certain embodiments, R₂ is propyl. In certain embodiments, R₂ is butyl. In certain embodiments, R₂ is F. In certain embodiments, R₂ is —CN. In certain embodiments, R₂ is —NO₂. In certain embodiments, R₂ is —OR_{A}. In certain embodiments, R₂ is -OC(═O)R_{A}. In certain embodiments, R₂ is —OC(═O)R_{A}, wherein R_{A} is aryl. In certain embodiments, R₂ is —OC(═O)R_{A}, wherein R_{A} is 4-nitrophenyl.

In some embodiments

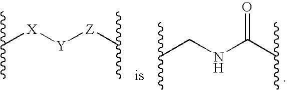

In some embodiments

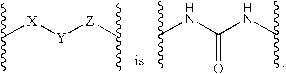

In some embodiments

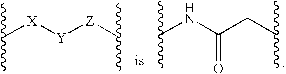

In some embodiments

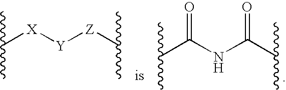

In some embodiments

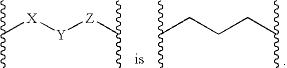

In some embodiments, the HDAC1 activator is

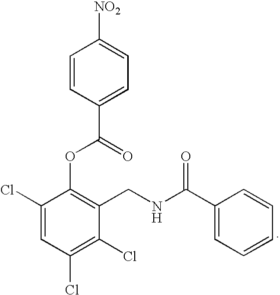

In some embodiments the HDAC activator is one of molecules 1-24, which are depicted below:

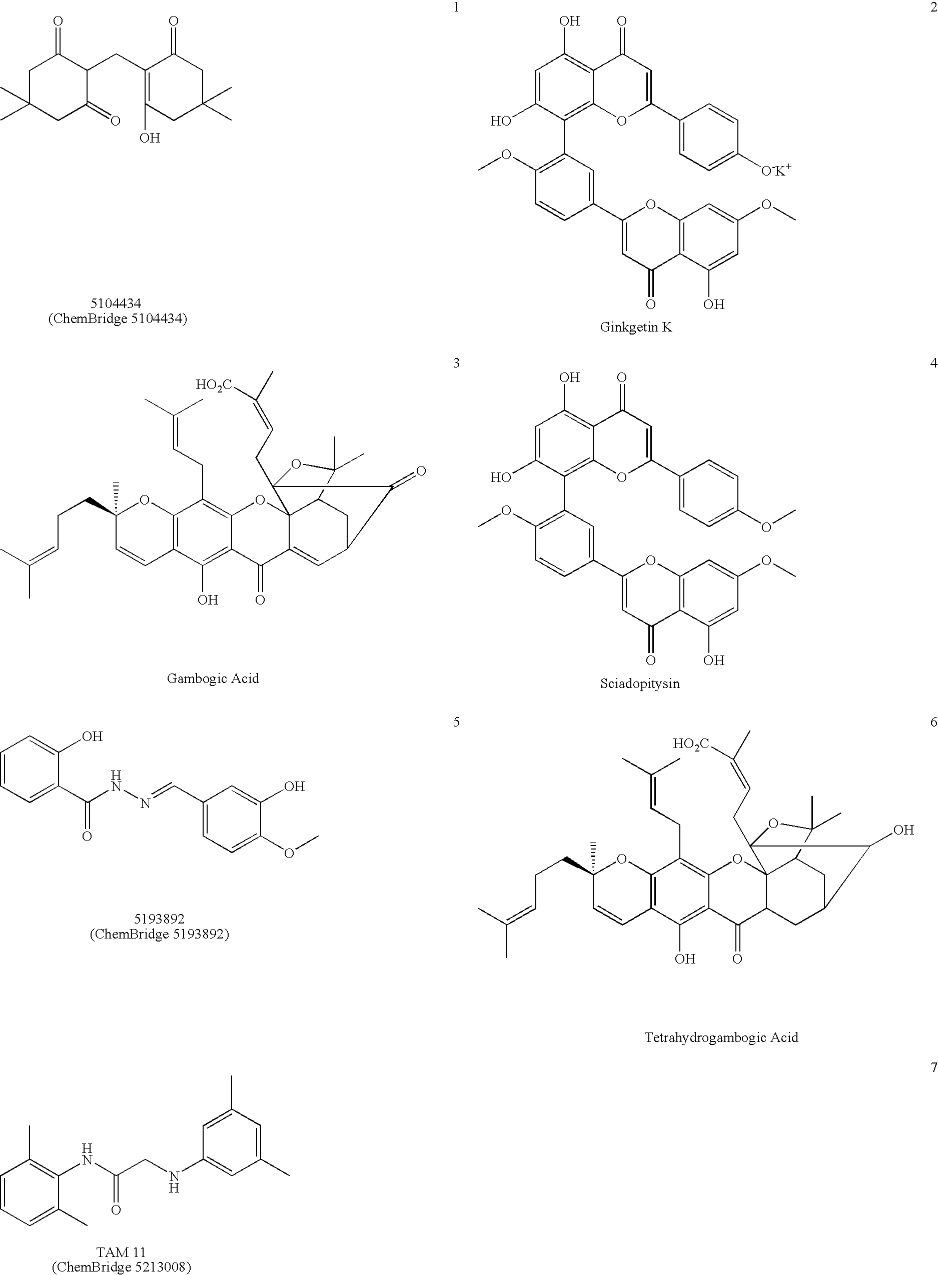

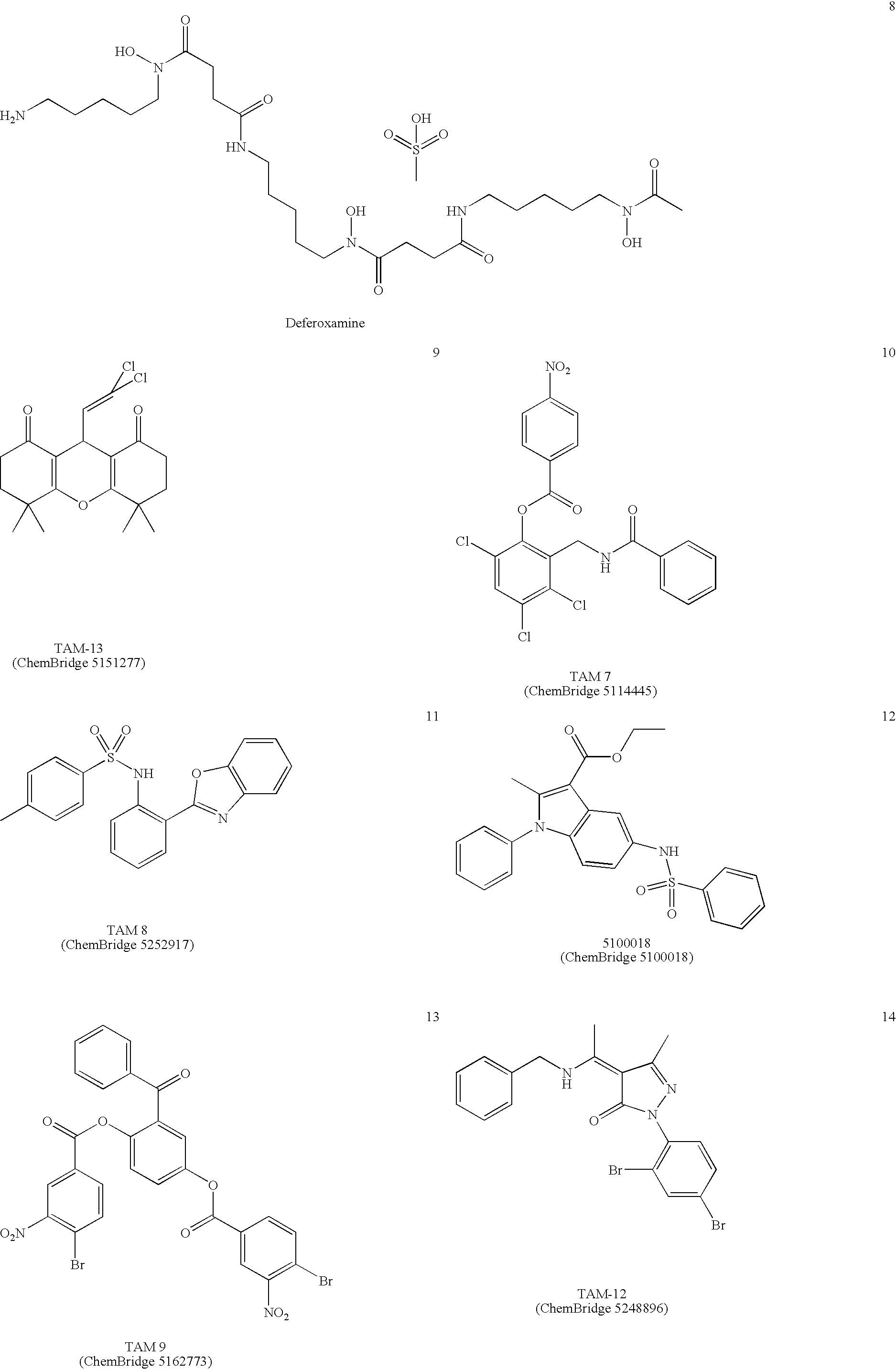

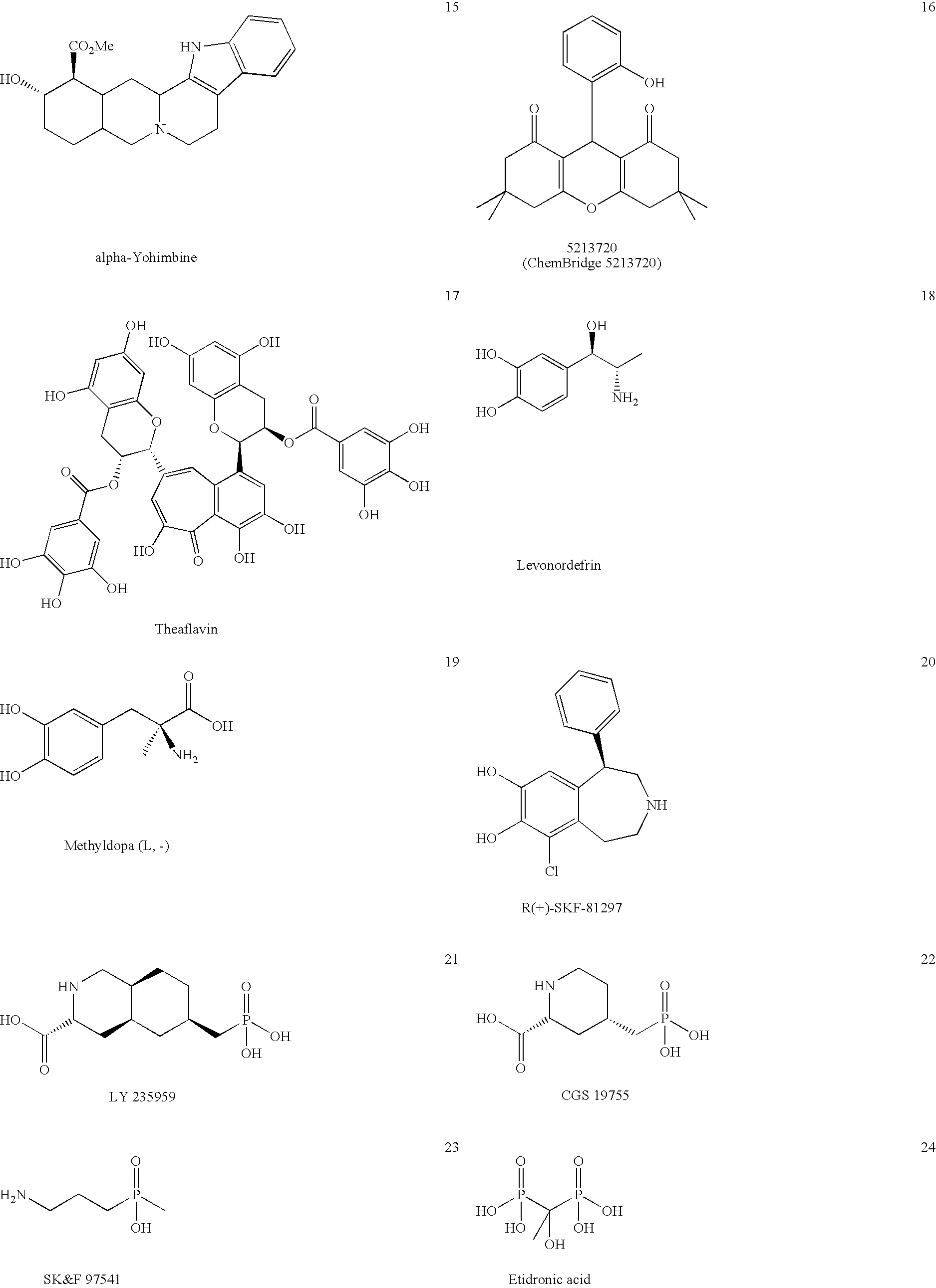

In some embodiments, the HDAC activator is a catechol derivative. Examples of catechol derivatives suitable for use in the present invention include those listed in U.S. Pat. Nos. 4,086,265, 5,013,756, 5,025,036, 5,102,906, 3,939,253, 3,998,799, 4,035,507, 4,125,519, 6,150,412, 5,633,371, 5,614,346, 5,489,614, 5,476,875, 5,389,653, 5,236,952, and 5,362,733, the entirety of which are incorporated herein by reference.

In some embodiments, the HDAC activator is a phosphorus-containing compound. Examples of phosphorus-containing compounds suitable for use in the present invention include those listed in U.S. Pat. No. 7,528,280, the entirety of which is incorporated herein by reference.

In some embodiments the HDAC activator is a metal chelator. Examples of metal chelators suitable for use in the present invention include those listed in U.S. Pat. Nos. 5,430,038, 5,430,176, and 5,011,976, the entirety of which are incorporated herein by reference.

In addition, the invention embraces HAT (histone acetyl transferases) inhibitors. Histone acetyl transferase inhibitors are known in the art and are described for instance in Eliseeva et al. (Eliseeva E D, Valkov V, Jung M, Jung M O. Characterization of novel inhibitors of histone acetyltransferases. Mol Cancer Ther. 2007 September;6(9):2391-8). Furthermore, one of ordinary skill in the art can select suitable compounds on the basis of the known structures of histone acetyl transferases. Examples of such compounds are peptides, nucleic acids expressing such peptides, small molecules etc, each of which can be naturally occurring molecules, synthetic molecules and/or FDA approved molecules, that specifically react with the histone acetyl transferase and suppress or inhibit its activity Histone acetyl transferases inhibitors also include expression inhibitors such as antisense and siRNA.

Definitions of specific functional groups and chemical terms are described in more detail below. For purposes of this invention, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; Carruthers, Some Modern Methods of Organic Synthesis, 3^{rd} Edition, Cambridge University Press, Cambridge, 1987.

The compounds of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention.

Where an isomer/enantiomer is preferred, it may, in some embodiments, be provided substantially free of the corresponding enantiomer, and may also be referred to as “optically enriched.” “Optically enriched,” as used herein, means that the compound is made up of a significantly greater proportion of one enantiomer. In certain embodiments the compound of the present invention is made up of at least about 90% by weight of a preferred enantiomer. In other embodiments the compound is made up of at least about 95%, 98%, or 99% by weight of a preferred enantiomer. Preferred enantiomers may be isolated from racemic mixtures by any method known to those skilled in the art, including chiral high pressure liquid chromatography (HPLC) and the formation and crystallization of chiral salts or prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); Wilen, Tables of Resolving Agents and Optical Resolutions p. 268 (E. L. Eliel, Ed., Univ. of Notre Dame Press, Notre Dame, Ind. 1972).

It will be appreciated that the compounds of the present invention, as described herein, may be substituted with any number of substituents or functional moieties. In general, the term “substituted” whether preceded by the term “optionally” or not, and substituents contained in formulas of this invention, refer to the replacement of hydrogen radicals in a given structure with the radical of a specified substituent. When more than one position in any given structure may be substituted with more than one substituent selected from a specified group, the substituent may be either the same or different at every position. As used herein, the term “substituted” is contemplated to include substitution with all permissible substituents of organic compounds, any of the substituents described herein (for example, aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, etc.), and any combination thereof (for example, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like) that results in the formation of a stable moiety. The present invention contemplates any and all such combinations in order to arrive at a stable substituent/moiety. Additional examples of generally applicable substitutents are illustrated by the specific embodiments described herein. For purposes of this invention, heteroatoms such as nitrogen may have hydrogen substituents and/or any suitable substituent as described herein which satisfy the valencies of the heteroatoms and results in the formation of a stable moiety.

The term “acyl,” as used herein, refers to a group having the general formula —C(═O)R^{X1}, —C(═O)OR^{X1}, —C(═O)—O—C(═O)R^{X1}, —C(═O)SR^{X1}, —C(═O)N(R^{X1})₂, —C(═S)R^{X1}, —C(═S)N(R^{X1})₂, and —C(═S)S(R^{X1}), —C(═NR^{X1})R^{X1}, —C(═NR^{X1})OR^{X1}, —C(═NR^{X1})SR^{X1}, and —C(═NR^{X1})N(R^{X1})₂, wherein R^{X1} is hydrogen; halogen; substituted or unsubstituted hydroxyl; substituted or unsubstituted thiol; substituted or unsubstituted amino; substituted or unsubstituted acyl, cyclic or acyclic, substituted or unsubstituted, branched or unbranched aliphatic; cyclic or acyclic, substituted or unsubstituted, branched or unbranched heteroaliphatic; cyclic or acyclic, substituted or unsubstituted, branched or unbranched alkyl; cyclic or acyclic, substituted or unsubstituted, branched or unbranched alkenyl; substituted or unsubstituted alkynyl; substituted or unsubstituted aryl, substituted or unsubstituted heteroaryl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, mono- or di-aliphaticamino, mono- or di-heteroaliphaticamino, mono- or di-alkylamino, mono- or di-heteroalkylamino, mono- or di-arylamino, or mono- or di-heteroarylamino; or two R^{X1} groups taken together form a 5- to 6-membered heterocyclic ring. Exemplary acyl groups include aldehydes (—CHO), carboxylic acids (—CO₂H), ketones, acyl halides, esters, amides, imines, carbonates, carbamates, and ureas. Acyl substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The term “acetyl,” (Ac) as used herein, refers to a group —C(═O)CH₃.

The term “acyloxy” refers to a “substituted hydroxyl” of the formula (—ORⁱ), wherein Rⁱ is an optionally substituted acyl group, as defined herein, and the oxygen moiety is directly attached to the parent molecule.

The term “aliphatic,” as used herein, includes both saturated and unsaturated, straight chain (i.e., unbranched), branched, acyclic, and cyclic (i.e., carbocyclic) hydrocarbons, which are optionally substituted with one or more functional groups. As will be appreciated by one of ordinary skill in the art, “aliphatic” is intended herein to include, but is not limited to, alkyl, alkenyl, alkynyl, cycloalkyl, cycloalkenyl, and cycloalkynyl moieties. Thus, as used herein, the term “alkyl” includes straight, branched and cyclic alkyl groups. An analogous convention applies to other generic terms such as “alkenyl”, “alkynyl”, and the like. Furthermore, as used herein, the terms “alkyl”, “alkenyl”, “alkynyl”, and the like encompass both substituted and unsubstituted groups. In certain embodiments, as used herein, “aliphatic” is used to indicate those aliphatic groups (cyclic, acyclic, substituted, unsubstituted, branched or unbranched) having 1-20 carbon atoms. Aliphatic group substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or to may not be further substituted).

The term “alkyl,” as used herein, refers to saturated, straight- or branched-chain hydrocarbon radicals derived from a hydrocarbon moiety containing between one and twenty carbon atoms by removal of a single hydrogen atom. In some embodiments, the alkyl group employed in the invention contains 1-20 carbon atoms. In another embodiment, the alkyl group employed contains 1-15 carbon atoms. In another embodiment, the alkyl group employed contains 1-10 carbon atoms. In another embodiment, the alkyl group employed contains 1-8 carbon atoms. In another embodiment, the alkyl group employed contains 1-5 carbon atoms. Examples of alkyl radicals include, but are not limited to, methyl, ethyl, n-propyl, isopropyl, n-butyl, iso-butyl, sec-butyl, sec-pentyl, iso-pentyl, tert-butyl, n-pentyl, neopentyl, n-hexyl, sec-hexyl, n-heptyl, n-octyl, n-decyl, n-undecyl, dodecyl, and the like, which may bear one or more sustitutents. Alkyl group substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The term “alkenyl,” as used herein, denotes a monovalent group derived from a straight- or branched-chain hydrocarbon moiety having at least one carbon-carbon double bond by the removal of a single hydrogen atom. In certain embodiments, the alkenyl group employed in the invention contains 2-20 carbon atoms. In some embodiments, the alkenyl group employed in the invention contains 2-15 carbon atoms. In another embodiment, the alkenyl group employed contains 2-10 carbon atoms. In still other embodiments, the alkenyl group contains 2-8 carbon atoms. In yet other embodiments, the alkenyl group contains 2-5 carbons. Alkenyl groups include, for example, ethenyl, propenyl, butenyl, 1-methyl-2-buten-1-yl, and the like, which may bear one or more substituents. Alkenyl group substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, to hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The term “alkynyl,” as used herein, refers to a monovalent group derived from a straight- or branched-chain hydrocarbon having at least one carbon-carbon triple bond by the removal of a single hydrogen atom. In certain embodiments, the alkynyl group employed in the invention contains 2-20 carbon atoms. In some embodiments, the alkynyl group employed in the invention contains 2-15 carbon atoms. In another embodiment, the alkynyl group employed contains 2-10 carbon atoms. In still other embodiments, the alkynyl group contains 2-8 carbon atoms. In still other embodiments, the alkynyl group contains 2-5 carbon atoms. Representative alkynyl groups include, but are not limited to, ethynyl, 2-propynyl(propargyl), 1-propynyl, and the like, which may bear one or more substituents. Alkynyl group substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The term “amino,” as used herein, refers to a group of the formula (—NH₂). A “substituted amino” refers either to a mono-substituted amine (—NHR^{h}) of a disubstitued amine (—NR^{h}₂), wherein the R^{h} substituent is any substitutent as described herein that results in the formation of a stable moiety (e.g., a suitable amino protecting group; aliphatic, alkyl, alkenyl, alkynyl, heteroaliphatic, heterocyclic, aryl, heteroaryl, acyl, amino, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or to may not be further substituted). In certain embodiments, the R^{h} substituents of the di-substituted amino group(—NR^{h}₂) form a 5- to 6-membered hetereocyclic ring.

The term “alkoxy” refers to a “substituted hydroxyl” of the formula (—ORⁱ), wherein Rⁱ is an optionally substituted alkyl group, as defined herein, and the oxygen moiety is directly attached to the parent molecule.

The term “alkylamino” refers to a “substituted amino” of the formula (—NR^{h}₂), wherein R^{h} is, independently, a hydrogen or an optionally subsituted alkyl group, as defined herein, and the nitrogen moiety is directly attached to the parent molecule.

The term “aryl,” as used herein, refer to stable aromatic mono- or polycyclic ring system having 3-20 ring atoms, of which all the ring atoms are carbon, and which may be substituted or unsubstituted. In certain embodiments of the present invention, “aryl” refers to a mono, bi, or tricyclic C₄-C₂₀ aromatic ring system having one, two, or three aromatic rings which include, but not limited to, phenyl, biphenyl, naphthyl, and the like, which may bear one or more substituents. Aryl substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The term “azido,” as used herein, refers to a group of the formula (—N₃).

The term “cyano,” as used herein, refers to a group of the formula (—CN).

The terms “halo” and “halogen” as used herein refer to an atom selected from fluorine (fluoro, —F), chlorine (chloro, —Cl), bromine (bromo, —Br), and iodine (iodo, —I).

The term “heteroaliphatic,” as used herein, refers to an aliphatic moiety, as defined herein, which includes both saturated and unsaturated, nonaromatic, straight chain (i.e., unbranched), branched, acyclic, cyclic (i.e., heterocyclic), or polycyclic hydrocarbons, which are optionally substituted with one or more functional groups, and that contain one or more oxygen, sulfur, nitrogen, phosphorus, or silicon atoms, e.g., in place of carbon atoms. In certain embodiments, heteroaliphatic moieties are substituted by independent replacement of one or more of the hydrogen atoms thereon with one or more substituents. As will be to appreciated by one of ordinary skill in the art, “heteroaliphatic” is intended herein to include, but is not limited to, heteroalkyl, heteroalkenyl, heteroalkynyl, heterocycloalkyl, heterocycloalkenyl, and heterocycloalkynyl moieties. Thus, the term “heteroaliphatic” includes the terms “heteroalkyl,” “heteroalkenyl”, “heteroalkynyl”, and the like. Furthermore, as used herein, the terms “heteroalkyl”, “heteroalkenyl”, “heteroalkynyl”, and the like encompass both substituted and unsubstituted groups. In certain embodiments, as used herein, “heteroaliphatic” is used to indicate those heteroaliphatic groups (cyclic, acyclic, substituted, unsubstituted, branched or unbranched) having 1-20 carbon atoms. Heteroaliphatic group substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, sulfinyl, sulfonyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The term “heteroalkyl,” as used herein, refers to an alkyl moiety, as defined herein, which contain one or more oxygen, sulfur, nitrogen, phosphorus, or silicon atoms, e.g., in place of carbon atoms.

The term “heterocyclic,” “heterocycles,” or “heterocyclyl,” as used herein, refers to a cyclic heteroaliphatic group. A heterocyclic group refers to a non-aromatic, partially unsaturated or fully saturated, 3- to 10-membered ring system, which includes single rings of 3 to 8 atoms in size, and bi- and tri-cyclic ring systems which may include aromatic five- or six-membered aryl or heteroaryl groups fused to a non-aromatic ring. These heterocyclic rings include those having from one to three heteroatoms independently selected from oxygen, sulfur, and nitrogen, in which the nitrogen and sulfur heteroatoms may optionally be oxidized and the nitrogen heteroatom may optionally be quaternized. In certain embodiments, the term heterocylic refers to a non-aromatic 5-, 6-, or 7-membered ring or polycyclic group wherein at least one ring atom is a heteroatom selected from O, S, and N (wherein the nitrogen and sulfur heteroatoms may be optionally oxidized), and the remaining ring atoms are carbon, the radical being joined to the rest of the molecule via any of the ring atoms. Heterocycyl groups include, but are not limited to, a bi- or tri-cyclic group, to comprising fused five, six, or seven-membered rings having between one and three heteroatoms independently selected from the oxygen, sulfur, and nitrogen, wherein (i) each 5-membered ring has 0 to 2 double bonds, each 6-membered ring has 0 to 2 double bonds, and each 7-membered ring has 0 to 3 double bonds, (ii) the nitrogen and sulfur heteroatoms may be optionally oxidized, (iii) the nitrogen heteroatom may optionally be quaternized, and (iv) any of the above heterocyclic rings may be fused to an aryl or heteroaryl ring.

Exemplary heterocycles include azacyclopropanyl, azacyclobutanyl, 1,3-diazatidinyl, piperidinyl, piperazinyl, azocanyl, thiaranyl, thietanyl, tetrahydrothiophenyl, dithiolanyl, thiacyclohexanyl, oxiranyl, oxetanyl, tetrahydrofuranyl, tetrahydropuranyl, dioxanyl, oxathiolanyl, morpholinyl, thioxanyl, tetrahydronaphthyl, and the like, which may bear one or more substituents. Substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, sulfinyl, sulfonyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The term “heteroaryl,” as used herein, refer to stable aromatic mono- or polycyclic ring system having 3-20 ring atoms, of which one ring atom is selected from S, O, and N; zero, one, or two ring atoms are additional heteroatoms independently selected from S, O, and N; and the remaining ring atoms are carbon, the radical being joined to the rest of the molecule via any of the ring atoms. Exemplary heteroaryls include, but are not limited to pyrrolyl, pyrazolyl, imidazolyl, pyridinyl, pyrimidinyl, pyrazinyl, pyridazinyl, triazinyl, tetrazinyl, pyyrolizinyl, indolyl, quinolinyl, isoquinolinyl, benzoimidazolyl, indazolyl, quinolinyl, isoquinolinyl, quinolizinyl, cinnolinyl, quinazolynyl, phthalazinyl, naphthridinyl, quinoxalinyl, thiophenyl, thianaphthenyl, furanyl, benzofuranyl, benzothiazolyl, thiazolynyl, isothiazolyl, thiadiazolynyl, oxazolyl, isoxazolyl, oxadiaziolyl, oxadiaziolyl, and the like, which may bear one or more substituents. Heteroaryl substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, sulfinyl, sulfonyl, oxo, imino, thiooxo, cyano, isocyano, amino, azido, nitro, hydroxyl, thiol, to halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The term “heteroarylamino” refers to a “substituted amino” of the (—NR^{h}₂), wherein R^{h} is, independently, a hydrogen or an optionally substituted heteroaryl group, as defined herein, and the nitrogen moiety is directly attached to the parent molecule.

The term “heteroaryloxy” refers to a “substituted hydroxyl” of the formula (—OR¹), wherein R¹ is an optionally substituted heteroaryl group, as defined herein, and the oxygen moiety is directly attached to the parent molecule.

The term “hydroxy,” or “hydroxyl,” as used herein, refers to a group of the formula (—OH). A “substituted hydroxyl” refers to a group of the formula (—ORⁱ), wherein Rⁱ can be any substitutent which results in a stable moiety (e.g., a suitable hydroxyl protecting group; aliphatic, alkyl, alkenyl, alkynyl, heteroaliphatic, heterocyclic, aryl, heteroaryl, acyl, nitro, alkylaryl, arylalkyl, and the like, each of which may or may not be further substituted).

The term “imino,” as used herein, refers to a group of the formula (═NR^{r}), wherein R^{r} corresponds to hydrogen or any substitutent as described herein, that results in the formation of a stable moiety (for example, a suitable amino protecting group; aliphatic, alkyl, alkenyl, alkynyl, heteroaliphatic, heterocyclic, aryl, heteroaryl, acyl, amino, hydroxyl, alkylaryl, arylalkyl, and the like, each of which may or may not be further substituted). In certain embodiments, imino refers to ═NH wherein R^{r} is hydrogen.

The term “nitro,” as used herein, refers to a group of the formula (—NO₂).

The term “oxo,” as used herein, refers to a group of the formula (═O).

A “protecting group” (P_{G}) as used herein, is well known in the art and include those described in detail in Protecting Groups in Organic Synthesis, T. W. Greene and P. G. M. Wuts, 3^{rd} edition, John Wiley & Sons, 1999, the entirety of which is incorporated herein by reference. “Suitable amino protecting groups” include methyl carbamate, ethyl carbamante, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, phenothiazinyl-(10)-carbonyl derivative, N′-p-toluensulfonylaminocarbonyl derivative, N′-phenylaminothiocarbonyl derivative, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxycarbonylvinyl carbamate, o-(N,N-dimethylcarboxamido)benzyl carbamate, 1,1-dimethyl-3//(NN-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, 2,4,6-trimethylbenzyl carbamate, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxycarbonylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide, o-(benzoyloxymethyl)benzamide, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxylmethylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[2-pyridyl)mesityllmethyleneamine, N-(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleamine, N-cyclohexylideneamine, N-(5,5-dimethyl-3-oxo-1-cyclohexenyeamine, N-borane derivative, N-diphenylborinic acid derivative, N-[phenyl(pentacarbonylchromium- or tungsten)carbonyll amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, 3-nitropyridinesulfenamide (Npys), p-toluenesulfonamide (Ts), benzenesulfonamide, 2,3,6,-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide

A “suitable carboxylic acid protecting group,” or “protected carboxylic acid,” as used herein, are well known in the art and include those described in detail in Greene (1999). Examples of suitably protected carboxylic acids further include, but are not limited to, silyl-, alkyl-, alkenyl-, aryl-, and arylalkyl-protected carboxylic acids. Examples of suitable silyl groups include trimethylsilyl, triethylsilyl, t-butyldimethylsilyl, t-butyldiphenylsilyl, triisopropylsilyl, and the like. Examples of suitable alkyl groups include methyl, benzyl, p-methoxybenzyl, 3,4-dimethoxybenzyl, trityl, t-butyl, tetrahydropyran-2-yl. Examples of suitable alkenyl groups include allyl. Examples of suitable aryl groups include optionally substituted phenyl, biphenyl, or naphthyl. Examples of suitable arylalkyl groups include optionally substituted benzyl (e.g., p-methoxybenzyl (MPM), 3,4-dimethoxybenzyl, O-nitrobenzyl, p-nitrobenzyl, p-halobenzyl, 2,6-dichlorobenzyl, p-cyanobenzyl), and 2- and 4-picolyl.

A “suitable hydroxyl protecting group” as used herein, is well known in the art and include those described in detail in Greene (1999). Suitable hydroxyl protecting groups include methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl, p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′- bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl)ethyl carbonate (Psec), 2-(triphenylphosphonio)ethyl carbonate (Peoc), alkyl isobutyl carbonate, alkyl vinyl carbonate alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2- to (methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxycarbonyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts). For protecting 1,2- or 1,3-diols, the protecting groups include methylene acetal, ethylidene acetal, 1-t-butylethylidene ketal, 1-phenylethylidene ketal, (4-methoxyphenyl)ethylidene acetal, 2,2,2-trichloroethylidene acetal, acetonide, cyclopentylidene ketal, cyclohexylidene ketal, cycloheptylidene ketal, benzylidene acetal, p-methoxybenzylidene acetal, 2,4-dimethoxybenzylidene ketal, 3,4-dimethoxybenzylidene acetal, 2-nitrobenzylidene acetal, methoxymethylene acetal, ethoxymethylene acetal, dimethoxymethylene ortho ester, 1-methoxyethylidene ortho ester, 1-ethoxyethylidine ortho ester, 1,2-dimethoxyethylidene ortho ester, α-methoxybenzylidene ortho ester, 1-(N,N-dimethylamino)ethylidene derivative, α-(N,N′-dimethylamino)benzylidene derivative, 2-oxacyclopentylidene ortho ester, di-t-butylsilylene group (DTBS), 1,3-(1,1,3,3-tetraisopropyldisiloxanylidene) derivative (TIPDS), tetra-t-butoxydisiloxane-1,3-diylidene derivative (TBDS), cyclic carbonates, cyclic boronates, ethyl boronate, and phenyl boronate.

As used herein, the term “pharmaceutically acceptable salt” refers to those salts which are, within the scope of sound medical judgment, suitable for use in contact with the tissues of humans and lower animals without undue toxicity, irritation, immunological response, and the like, and are commensurate with a reasonable benefit/risk ratio. Pharmaceutically acceptable salts are well known in the art. For example, Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C_{1 4}alky)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate, and aryl sulfonate.

As used herein, the term “treating” and “treatment” refers to administering a compound to a subject and/or performing an action on a subject so that the subject has an improvement in the disease or disorder, for example, beneficial or desired clinical results. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. One of skill in the art realizes that a treatment may improve the disease condition, but may not be a complete cure for the disease. As used herein, the phrase “protecting against neuronal damage” means decreasing the incidence or severity of neuronal damage through prophylactic action, for instance the administration of a specific compound.

The terms “effective amount” and “therapeutically effective amount,” as used herein, refer to the amount or concentration of an inventive compound, that, when administered to a subject, is effective to at least partially treat a condition from which the subject is suffering.

A subject shall mean a human or vertebrate animal or mammal including but not limited to a dog, cat, horse, cow, pig, sheep, goat, turkey, chicken, and primate, e.g., monkey. In some embodiments, subjects are those which are not otherwise in need of an HDAC activator.

The term “neurological disorder” as used in this invention includes neurological diseases, neurodegenerative diseases and neuropsychiatric disorders. A neurological disorder is a condition having as a component a central or peripheral nervous system malfunction. Neurological disorders may cause a disturbance in the structure or function of the nervous system resulting from developmental abnormalities, disease, genetic defects, injury or toxin. These disorders may affect the central nervous system (e.g., the brain, brainstem and cerebellum), the peripheral nervous system (e.g., the cranial nerves, spinal nerves, and sympathetic and parasympathetic nervous systems) and/or the autonomic nervous system (e.g., the part of the nervous system that regulates involuntary action and that is divided into the sympathetic and parasympathetic nervous systems).

As used herein, the term “neurodegenerative disease” implies any disorder that might be reversed, deterred, managed, treated, improved, or eliminated with agents that stimulate the generation of new neurons. Examples of neurodegenerative disorders include: (i) chronic neurodegenerative diseases such as familial and sporadic amyotrophic lateral sclerosis (FALS and ALS, respectively), familial and sporadic Parkinson's disease, Huntington's disease, familial and sporadic Alzheimer's disease, multiple sclerosis, olivopontocerebellar atrophy, multiple system atrophy, progressive supranuclear palsy, diffuse Lewy body disease, corticodentatonigral degeneration, progressive familial myoclonic epilepsy, strionigral degeneration, torsion dystonia, familial tremor, Down's Syndrome, Gilles de la Tourette syndrome, Hallervorden-Spatz disease, diabetic peripheral neuropathy, dementia pugilistica, AIDS Dementia, age related dementia, age associated memory impairment, and amyloidosis-related neurodegenerative diseases such as those caused by the prion protein (PrP) which is associated with transmissible spongiform encephalopathy (Creutzfeldt-Jakob disease, Gerstmann-Straussler-Scheinker syndrome, scrapic, and kuru), and those caused by excess cystatin C accumulation (hereditary cystatin C angiopathy); and (ii) acute neurodegenerative disorders such as traumatic brain injury (e.g., surgery-related brain injury), cerebral edema, peripheral nerve damage, spinal cord injury, Leigh's disease, Guillain-Barre syndrome, lysosomal storage disorders such as lipofuscinosis, Alper's disease, vertigo as result of CNS degeneration; pathologies arising with chronic alcohol or drug abuse including, for example, the degeneration of neurons in locus coeruleus and cerebellum; pathologies arising with aging including degeneration of cerebellar neurons and cortical neurons leading to cognitive and motor impairments; and pathologies arising with chronic amphetamine abuse to including degeneration of basal ganglia neurons leading to motor impairments; pathological changes resulting from focal trauma such as stroke, focal ischemia, vascular insufficiency, hypoxic-ischemic encephalopathy, hyperglycemia, hypoglycemia or direct trauma; pathologies arising as a negative side-effect of therapeutic drugs and treatments (e.g., degeneration of cingulate and entorhinal cortex neurons in response to anticonvulsant doses of antagonists of the NMDA class of glutamate receptor) and Wernicke-Korsakoff s related dementia. Neurodegenerative diseases affecting sensory neurons include Friedreich's ataxia, diabetes, peripheral neuropathy, and retinal neuronal degeneration. Other neurodegenerative diseases include nerve injury or trauma associated with spinal cord injury. Neurodegenerative diseases of limbic and cortical systems include cerebral amyloidosis, Pick's atrophy, and Retts syndrome. The foregoing examples are not meant to be comprehensive but serve merely as an illustration of the term “neurodegenerative disorder.”

Parkinson's disease is a disturbance of voluntary movement in which muscles become stiff and sluggish. Symptoms of the disease include difficult and uncontrollable rhythmic twitching of groups of muscles that produces shaking or tremors. The disease is caused by degeneration of pre-synaptic dopaminergic neurons in the brain and specifically in the brain stem. As a result of the degeneration, an inadequate release of the chemical transmitter dopamine occurs during neuronal activity. Currently, Parkinson's disease is treated with several different compounds and combinations. Levodopa (L-dopa), which is converted into dopamine in the brain, is often given to restore muscle control. Perindopril, an ACE inhibitor that crosses the blood-brain barrier, is used to improve patients' motor responses to L-dopa. Carbidopa is administered with L-dopa in order to delay the conversion of L-dopa to dopamine until it reaches the brain, and it also lessens the side effects of L-dopa. Other drugs used in Parkinson's disease treatment include dopamine mimickers Mirapex (pramipexole dihydrochloride) and Requip (ropinirole hydrochloride), and Tasmar (tolcapone), a COMT inhibitor that blocks a key enzyme responsible for breaking down levodopa before it reaches the brain.

Amyotrophic lateral sclerosis (ALS), also called Lou Gehrig's disease, is a progressive, fatal neurological disease. ALS occurs when specific nerve cells in the brain and spinal cord that control voluntary movement gradually degenerate and causes the muscles under their control to weaken and waste away, leading to paralysis. Currently there is no cure for ALS; nor is there a proven therapy that will prevent or reverse the course of the disorder.

Autism (also referred to as Autism Spectrum Disorder, or ASD) is a disorder that seriously impairs the functioning of individuals. It is characterized by self-absorption, a reduced ability to communicate with or respond to the outside world, rituals and compulsive phenomena, and mental retardation. Autistic individuals are also at increased risk of developing seizure disorders, such as epilepsy. While the actual cause of autism is unknown, it appears to include one or more genetic factors, as indicated by the fact that the concordance rate is higher in monozygotic twins than in dizygotic twins, and may also involve immune and environmental factors, such as diet, toxic chemicals and infections.

In some instances the neurological disorder is a neuropsychiatric disorder, which refers to conditions or disorders that relate to the functioning of the brain and the cognitive processes or behavior. Neuropsychiatric disorders may be further classified based on the type of neurological disturbance affecting the mental faculties. The term “neuropsychiatric disorder,” considered here as a subset of “neurological disorders,” refers to a disorder which may be generally characterized by one or more breakdowns in the adaptation process. Such disorders are therefore expressed primarily in abnormalities of thought, feeling and/or behavior producing either distress or impairment of function (i.e., impairment of mental function such with dementia or senility). Currently, individuals may be evaluated for various neuropsychiatric disorders using criteria set forth in the most recent version of the American Psychiatric Association's Diagnostic and Statistical Manual of Mental Health (DSM-IV).

One group of neuropsychiatric disorders includes disorders of thinking and cognition, such as schizophrenia and delirium. A second group of neuropsychiatric disorders includes disorders of mood, such as affective disorders and anxiety. A third group of neuropsychiatric disorders includes disorders of social behavior, such as character defects and personality disorders. A fourth group of neuropsychiatric disorders includes disorders of learning, memory, and intelligence, such as mental retardation and dementia. Accordingly, neuropsychiatric disorders encompass schizophrenia, delirium, attention deficit disorder (ADD), schizoaffective disorder Alzheimer's disease, depression, mania, attention deficit disorders, drug addiction, dementia, agitation, apathy, anxiety, psychoses, personality disorders, bipolar disorders, unipolar affective disorder, obsessive-compulsive disorders, eating disorders, post-traumatic stress disorders, irritability, adolescent conduct disorder and disinhibition.

Schizophrenia is a disorder that affects about one percent of the world population. Three general symptoms of schizophrenia are often referred to as positive symptoms, negative symptoms, and disorganized symptoms. Positive symptoms can include delusions (abnormal beliefs), hallucinations (abnormal perceptions), and disorganized thinking. The hallucinations of schizophrenia can be auditory, visual, olfactory, or tactile. Disorganized thinking can manifest itself in schizophrenic patients by disjointed speech and the inability to maintain logical thought processes. Negative symptoms can represent the absence of normal behavior. Negative symptoms include emotional flatness or lack of expression and can be characterized by social withdrawal, reduced energy, reduced motivation, and reduced activity. Catatonia can also be associated with negative symptoms of schizophrenia. The symptoms of schizophrenia should continuously persist for a duration of about six months in order for the patient to be diagnosed as schizophrenic. Based on the types of symptoms a patient reveals, schizophrenia can be categorized into subtypes including catatonic schizophrenia, paranoid schizophrenia, and disorganized schizophrenia.

Examples of antipsychotic drugs that may be used to treat schizophrenic patients include phenothizines, such as chlorpromazine and trifluopromazine; thioxanthenes, such as chlorprothixene; fluphenazine; butyropenones, such as haloperidol; loxapine; mesoridazine; molindone; quetiapine; thiothixene; trifluoperazine; perphenazine; thioridazine; risperidone; dibenzodiazepines, such as clozapine; and olanzapine. Although these agents may relieve the symptoms of schizophrenia, their administration can result in undesirable side effects including Parkinson's disease-like symptoms (tremor, muscle rigidity, loss of facial expression); dystonia; restlessness; tardive dyskinesia; weight gain; skin problems; dry mouth; constipation; blurred vision; drowsiness; slurred speech and agranulocytosis.

Mania is a sustained form of euphoria that affects millions of people in the United States who suffer from depression. Manic episodes can be characterized by an elevated, expansive, or irritable mood lasting several days, and is often accompanied by other symptoms, such as, over-activity, over-talkativeness, social intrusiveness, increased energy, pressure of ideas, grandiosity, distractibility, decreased need for sleep, and recklessness. Manic patients can also experience delusions and hallucinations.

Depressive disorders can involve serotonergic and noradrenergic neuronal systems based on current therapeutic regimes that target serotonin and noradrenalin receptors. Mania may results from an imbalance in certain chemical messengers within the brain. Administering phosphotidyl choline has been reported to alleviate the symptoms of mania.

Anxiety disorders are characterized by frequent occurrence of symptoms of fear including arousal, restlessness, heightened responsiveness, sweating, racing heart, increased blood pressure, dry mouth, a desire to run or escape, and avoidance behavior. Generalized anxiety persists for several months, and is associated with motor tension (trembling, twitching, muscle aches, restlessness); autonomic hyperactivity (shortness of breath, palpitations, increased heart rate, sweating, cold hands), and vigilance and scanning (feeling on edge, exaggerated startle response, difficult in concentrating). Benzodiazepines, which enhance the inhibitory effects of the gamma aminobutyric acid (GABA) type A receptor, are frequently used to treat anxiety. Buspirone is another effective anxiety treatment.

Alzheimer's disease is a degenerative brain disorder characterized by cognitive and noncognitive neuropsychiatric symptoms. Psychiatric symptoms are common in Alzheimer's disease, with psychosis (hallucinations and delusions) present in approximately fifty percent of affected patients. Similar to schizophrenia, positive psychotic symptoms are common in Alzheimer's disease. Delusions typically occur more frequently than hallucinations. Alzheimer's patients may also exhibit negative symptoms, such as disengagement, apathy, diminished emotional responsiveness, loss of volition, and decreased initiative. Indeed, antipsychotic agents that are used to relieve psychosis of schizophrenia are also useful in alleviating psychosis in Alzheimer's patients. As used herein, the term “dementia” refers to the loss, of cognitive and intellectual functions without impairment of perception or consciousness. Dementia is typically characterized by disorientation, impaired memory, judgment, and intellect, and a shallow labile affect.

Schizo-affective disorder describes a condition where both the symptoms of a mood disorder and schizophrenia are present. A person may manifest impairments in the perception or expression of reality, most commonly in the form of auditory hallucinations, paranoid or bizarre delusions or disorganized speech and thinking, as well as discrete manic and/or depressive episodes in the context of significant social or occupational dysfunction.

Mood disorders are typically characterized by pervasive, prolonged, and disabling exaggerations of mood and affect that are associated with behavioral, physiologic, cognitive, neurochemical and psychomotor dysfunctions. The major mood disorders include, but are not limited to major depressive disorder (also known as unipolar disorder), bipolar disorder to (also known as manic depressive illness or bipolar depression), dysthymic disorder.

The therapeutic compounds of the invention may be directly administered to the subject or may be administered in conjunction with a delivery device or vehicle. Delivery vehicles or delivery devices for delivering therapeutic compounds to surfaces have been described. The therapeutic compounds of the invention may be administered alone (e.g., in saline or buffer) or using any delivery vehicles known in the art. For instance the following delivery vehicles have been described: Cochleates; Emulsomes, ISCOMs; Liposomes; Live bacterial vectors (e.g., Salmonella, Escherichia coli, Bacillus calmatte-guerin, Shigella, Lactobacillus); Live viral vectors (e.g., Vaccinia, adenovirus, Herpes Simplex); Microspheres; Nucleic acid vaccines; Polymers; Polymer rings; Proteosomes; Sodium Fluoride; Transgenic plants; Virosomes; Virus-like particles. Other delivery vehicles are known in the art and some additional examples are provided below.

The term effective amount of a therapeutic compound of the invention refers to the amount necessary or sufficient to realize a desired biologic effect. For example, as discussed above, an effective amount of a therapeutic compounds of the invention is that amount sufficient to treat the neurological disorder. Combined with the teachings provided herein, by choosing among the various active compounds and weighing factors such as potency, relative bioavailability, patient body weight, severity of adverse side-effects and preferred mode of administration, an effective prophylactic or therapeutic treatment regimen can be planned which does not cause substantial toxicity and yet is entirely effective to treat the particular subject. The effective amount for any particular application can vary depending on such factors as the disease or condition being treated, the particular therapeutic compounds being administered the size of the subject, or the severity of the disease or condition. One of ordinary skill in the art can empirically determine the effective amount of a particular therapeutic compounds of the invention without necessitating undue experimentation. Compositions of the invention include compounds as described herein, or a pharmaceutically acceptable salt or hydrate thereof.

Subject doses of the compounds described herein for delivery typically range from about 0.1 μg to 10 mg per administration, which depending on the application could be given daily, weekly, or monthly and any other amount of time there between. The doses for these purposes may range from about 10 μg to 5 mg per administration, and most typically from about 100 μg to 1 mg, with 2-4 administrations being spaced days or weeks apart. In some to embodiments, however, parenteral doses for these purposes may be used in a range of 5 to 10,000 times higher than the typical doses described above.

In one embodiment, the composition is administered once daily at a dose of about 200-600 mg. In another embodiment, the composition is administered twice daily at a dose of about 200-400 mg. In another embodiment, the composition is administered twice daily at a dose of about 200-400 mg intermittently, for example three, four, or five days per week. In another embodiment, the composition is administered three times daily at a dose of about 100-250 mg. In one embodiment, the daily dose is 200 mg, which can be administered once-daily, twice-daily, or three-times daily. In one embodiment, the daily dose is 300 mg, which can be administered once-daily or twice-daily. In one embodiment, the daily dose is 400 mg, which can be administered once-daily or twice-daily. The HDAC activator can be administered in a total daily dose of up to 800 mg once, twice or three times daily, continuously (i.e., every day) or intermittently (e.g., 3-5 days a week).

For any compound described herein the therapeutically effective amount can be initially determined from animal models. A therapeutically effective dose can also be determined from human data for HDAC activators which have been tested in humans and for compounds which are known to exhibit similar pharmacological activities. Higher doses may be required for parenteral administration. The applied dose can be adjusted based on the relative bioavailability and potency of the administered compound. Adjusting the dose to achieve maximal efficacy based on the methods described above and other methods as are well-known in the art is well within the capabilities of the ordinarily skilled artisan.

The formulations of the invention are administered in pharmaceutically acceptable solutions, which may routinely contain pharmaceutically acceptable concentrations of salt, buffering agents, preservatives, compatible carriers, and optionally other therapeutic ingredients.

For use in therapy, an effective amount of the therapeutic compounds of the invention can be administered to a subject by any mode that delivers the therapeutic agent or compound to the desired surface, e.g., mucosal, systemic. Administering the pharmaceutical composition of the present invention may be accomplished by any means known to the skilled artisan. Preferred routes of administration include but are not limited to oral, parenteral, intramuscular, intranasal, sublingual, intratracheal, inhalation, ocular, vaginal, rectal and intracerebroventricular.

For oral administration, the therapeutic compounds of the invention can be formulated readily by combining the active compound(s) with pharmaceutically acceptable carriers well known in the art. Such carriers enable the compounds of the invention to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers, i.e. EDTA for neutralizing internal acid conditions or may be administered without any carriers.

Also specifically contemplated are oral dosage forms of the above component or components. The component or components may be chemically modified so that oral delivery of the derivative is efficacious. Generally, the chemical modification contemplated is the attachment of at least one moiety to the component molecule itself, where said moiety permits (a) inhibition of proteolysis; and (b) uptake into the blood stream from the stomach or intestine. Also desired is the increase in overall stability of the component or components and increase in circulation time in the body. Examples of such moieties include: polyethylene glycol, copolymers of ethylene glycol and propylene glycol, carboxymethyl cellulose, dextran, polyvinyl alcohol, polyvinyl pyrrolidone and polyproline. Abuchowski and Davis, 1981, “Soluble Polymer-Enzyme Adducts” In: Enzymes as Drugs, Hocenberg and Roberts, eds., Wiley-Interscience, New York, N.Y., pp. 367-383; Newmark, et al., 1982, J. Appl. Biochem. 4:185-189. Other polymers that could be used are poly-1,3-dioxolane and poly-1,3,6-tioxocane. Preferred for pharmaceutical usage, as indicated above, are polyethylene glycol moieties.

The location of release may be the stomach, the small intestine (the duodenum, the jejunum, or the ileum), or the large intestine. One skilled in the art has available formulations which will not dissolve in the stomach, yet will release the material in the duodenum or elsewhere in the intestine. Preferably, the release will avoid the deleterious effects of the stomach environment, either by protection of the therapeutic agent or by release of the biologically active material beyond the stomach environment, such as in the intestine.

To ensure full gastric resistance a coating impermeable to at least pH 5.0 is important. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and Shellac. These coatings may be used as mixed films.

A coating or mixture of coatings can also be used on tablets, which are not intended for protection against the stomach. This can include sugar coatings, or coatings which make the tablet easier to swallow. Capsules may consist of a hard shell (such as gelatin) for delivery of dry therapeutic i.e. powder; for liquid forms, a soft gelatin shell may be used. The shell material of cachets could be thick starch or other edible paper. For pills, lozenges, molded tablets or tablet triturates, moist massing techniques can be used.

The therapeutic can be included in the formulation as fine multi-particulates in the form of granules or pellets of particle size about 1 mm The formulation of the material for capsule administration could also be as a powder, lightly compressed plugs or even as tablets. The therapeutic could be prepared by compression.

Colorants and flavoring agents may all be included. For example, the therapeutic agent may be formulated (such as by liposome or microsphere encapsulation) and then further contained within an edible product, such as a refrigerated beverage containing colorants and flavoring agents.

One may dilute or increase the volume of the therapeutic with an inert material. These diluents could include carbohydrates, especially mannitol, a-lactose, anhydrous lactose, cellulose, sucrose, modified dextrans and starch. Certain inorganic salts may be also be used as fillers including calcium triphosphate, magnesium carbonate and sodium chloride. Some commercially available diluents are Fast-Flo, Emdex, STA-Rx 1500, Emcompress and Avicell.

Disintegrants may be included in the formulation of the therapeutic into a solid dosage form. Materials used as disintegrates include but are not limited to starch, including the commercial disintegrant based on starch, Explotab. Sodium starch glycolate, Amberlite, sodium carboxymethylcellulose, ultramylopectin, sodium alginate, gelatin, orange peel, acid to carboxymethyl cellulose, natural sponge and bentonite may all be used. Another form of the disintegrants are the insoluble cationic exchange resins. Powdered gums may be used as disintegrants and as binders and these can include powdered gums such as agar, Karaya or tragacanth. Alginic acid and its sodium salt are also useful as disintegrants.

Binders may be used to hold the therapeutic agent together to form a hard tablet and include materials from natural products such as acacia, tragacanth, starch and gelatin. Others include methyl cellulose (MC), ethyl cellulose (EC) and carboxymethyl cellulose (CMC). Polyvinyl pyrrolidone (PVP) and hydroxypropylmethyl cellulose (HPMC) could both be used in alcoholic solutions to granulate the therapeutic.

An anti-frictional agent may be included in the formulation of the therapeutic to prevent sticking during the formulation process. Lubricants may be used as a layer between the therapeutic and the die wall, and these can include but are not limited to; stearic acid including its magnesium and calcium salts, polytetrafluoroethylene (PTFE), liquid paraffin, vegetable oils and waxes. Soluble lubricants may also be used such as sodium lauryl sulfate, magnesium lauryl sulfate, polyethylene glycol of various molecular weights, Carbowax 4000 and 6000.

Glidants that might improve the flow properties of the drug during formulation and to aid rearrangement during compression might be added. The glidants may include starch, talc, pyrogenic silica and hydrated silicoaluminate.

To aid dissolution of the therapeutic into the aqueous environment a surfactant might be added as a wetting agent. Surfactants may include anionic detergents such as sodium lauryl sulfate, dioctyl sodium sulfosuccinate and dioctyl sodium sulfonate. Cationic detergents might be used and could include benzalkonium chloride or benzethomium chloride. The list of potential non-ionic detergents that could be included in the formulation as surfactants are lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants could be present in the formulation of the therapeutic agent either alone or as a mixture in different ratios.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

For buccal administration, the compositions may take the form of tablets or lozenges formulated in conventional manner

For administration by inhalation, the compounds for use according to the present invention may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

Also contemplated herein is pulmonary delivery of the therapeutic compounds of the invention. The therapeutic agent is delivered to the lungs of a mammal while inhaling and traverses across the lung epithelial lining to the blood stream. Other reports of inhaled molecules include Adjei et al., 1990, Pharmaceutical Research, 7:565-569; Adjei et al., 1990, International Journal of Pharmaceutics, 63:135-144 (leuprolide acetate); Braquet et al., 1989, Journal of Cardiovascular Pharmacology, 13 (suppl. 5):143-146 (endothelin-1); Hubbard et al., 1989, Annals of Internal Medicine, Vol. III, pp. 206-212 (al- antitrypsin); Smith et al., 1989, J. Clin. Invest. 84:1145-1146 (a-l-proteinase); Oswein et al., 1990, “Aerosolization of Proteins”, Proceedings of Symposium on Respiratory Drug Delivery II, Keystone, Colo., March, (recombinant human growth hormone); Debs et al., 1988, J. Immunol. 140:3482-3488 (interferon-g and tumor necrosis factor alpha) and Platz et al., U.S. Pat. No. 5,284,656 (granulocyte colony stimulating factor). A method and composition for pulmonary delivery of drugs for systemic effect is described in U.S. Pat. No. 5,451,569, issued Sep. 19, 1995 to Wong et al.

Contemplated for use in the practice of this invention are a wide range of mechanical devices designed for pulmonary delivery of therapeutic products, including but not limited to nebulizers, metered dose inhalers, and powder inhalers, all of which are familiar to those skilled in the art.

Some specific examples of commercially available devices suitable for the practice of this invention are the Ultravent nebulizer, manufactured by Mallinckrodt, Inc., St. Louis, Mo.; the Acorn II nebulizer, manufactured by Marquest Medical Products, Englewood, Colo.; the Ventolin metered dose inhaler, manufactured by Glaxo Inc., Research Triangle Park, N.C.; and the Spinhaler powder inhaler, manufactured by Fisons Corp., Bedford, Mass.

All such devices require the use of formulations suitable for the dispensing of therapeutic agent. Typically, each formulation is specific to the type of device employed and may involve the use of an appropriate propellant material, in addition to the usual diluents, and/or carriers useful in therapy. Also, the use of liposomes, microcapsules or microspheres, inclusion complexes, or other types of carriers is contemplated. Chemically modified therapeutic agent may also be prepared in different formulations depending on the type of chemical modification or the type of device employed.

Formulations suitable for use with a nebulizer, either jet or ultrasonic, will typically comprise therapeutic agent dissolved in water at a concentration of about 0.1 to 25 mg of biologically active compound per mL of solution. The formulation may also include a buffer and a simple sugar (e.g., for stabilization and regulation of osmotic pressure). The nebulizer formulation may also contain a surfactant, to reduce or prevent surface induced aggregation of the compound caused by atomization of the solution in forming the aerosol.

Formulations for use with a metered-dose inhaler device will generally comprise a finely divided powder containing the therapeutic agent suspended in a propellant with the aid of a surfactant. The propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2-tetrafluoroethane, or combinations thereof. Suitable surfactants include sorbitan trioleate and soya lecithin. Oleic acid may also be useful as a surfactant.

Formulations for dispensing from a powder inhaler device will comprise a finely divided dry powder containing therapeutic agent and may also include a bulking agent, such as lactose, sorbitol, sucrose, or mannitol in amounts which facilitate dispersal of the powder from the device, e.g., 50 to 90% by weight of the formulation. The therapeutic agent should most advantageously be prepared in particulate form with an average particle size of less than 10 mm (or microns), most preferably 0.5 to 5 mm, for most effective delivery to the distal lung.

Nasal delivery of a pharmaceutical composition of the present invention is also contemplated. Nasal delivery allows the passage of a pharmaceutical composition of the present invention to the blood stream directly after administering the therapeutic product to the nose, without the necessity for deposition of the product in the lung. Formulations for nasal delivery include those with dextran or cyclodextran.

For nasal administration, a useful device is a small, hard bottle to which a metered dose sprayer is attached. In one embodiment, the metered dose is delivered by drawing the pharmaceutical composition of the present invention solution into a chamber of defined volume, which chamber has an aperture dimensioned to aerosolize and aerosol formulation by forming a spray when a liquid in the chamber is compressed. The chamber is compressed to administer the pharmaceutical composition of the present invention. In a specific embodiment, the chamber is a piston arrangement. Such devices are commercially available.

Alternatively, a plastic squeeze bottle with an aperture or opening dimensioned to aerosolize an aerosol formulation by forming a spray when squeezed is used. The opening is usually found in the top of the bottle, and the top is generally tapered to partially fit in the nasal passages for efficient administration of the aerosol formulation. Preferably, the nasal inhaler will provide a metered amount of the aerosol formulation, for administration of a measured dose of the drug.

The compounds, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable to stabilizers or agents which increase the solubility of the compounds to allow for the preparation of highly concentrated solutions.

Alternatively, the active compounds may be in powder form for constitution with a suitable vehicle, e.g., sterile pyrogen-free water, before use.

The compounds may also be formulated in rectal or vaginal compositions such as suppositories or retention enemas, e.g., containing conventional suppository bases such as cocoa butter or other glycerides.

In addition to the formulations described previously, the compounds may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin The pharmaceutical compositions also include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see Langer, Science 249:1527-1533, 1990, which is incorporated herein by reference.

The therapeutic compounds of the invention and optionally other therapeutics may be administered per se (neat) or in the form of a pharmaceutically acceptable salt. When used in medicine the salts should be pharmaceutically acceptable, but non-pharmaceutically acceptable salts may conveniently be used to prepare pharmaceutically acceptable salts to thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

The pharmaceutical compositions of the invention contain an effective amount of a therapeutic compound of the invention optionally included in a pharmaceutically-acceptable carrier. The term pharmaceutically-acceptable carrier means one or more compatible solid or liquid filler, diluents or encapsulating substances which are suitable for administration to a human or other vertebrate animal. The term carrier denotes an organic or inorganic ingredient, natural or synthetic, with which the active ingredient is combined to facilitate the application. The components of the pharmaceutical compositions also are capable of being commingled with the compounds of the present invention, and with each other, in a manner such that there is no interaction which would substantially impair the desired pharmaceutical efficiency.

The therapeutic agents may be delivered to the brain using a formulation capable of delivering a therapeutic agent across the blood brain barrier. One obstacle to delivering therapeutics to the brain is the physiology and structure of the brain. The blood-brain barrier is made up of specialized capillaries lined with a single layer of endothelial cells. The region between cells are sealed with a tight junction, so the only access to the brain from the blood is through the endothelial cells. The barrier allows only certain substances, such as lipophilic molecules through and keeps other harmful compounds and pathogens out. Thus, lipophilic carriers are useful for delivering non-lipohilic compounds to the brain. For instance, DHA, a fatty acid naturally occurring in the human brain has been found to be useful for delivering drugs covalently attached thereto to the brain (Such as those described in U.S. Pat. No. 6407137). U.S. Pat. No. 5,525,727 describes a dihydropyridine pyridinium salt carrier redox system for the specific and sustained delivery of drug species to the brain. U.S. Pat. No. 5,618,803 describes targeted drug delivery with phosphonate derivatives. U.S. Pat. No. 7119074 to describes amphiphilic prodrugs of a therapeutic compound conjugated to an PEG-oligomer/polymer for delivering the compound across the blood brain barrier. The compounds described herein may be modified by covalent attachment to a lipophilic carrier or co-formulation with a lipophilic carrier. Others are known to those of skill in the art.

The agents described herein may, in some embodiments, be assembled into pharmaceutical or diagnostic or research kits to facilitate their use in therapeutic, diagnostic or research applications. A kit may include one or more containers housing the components of the invention and instructions for use. Specifically, such kits may include one or more agents described herein, along with instructions describing the intended therapeutic application and the proper administration of these agents. In certain embodiments agents in a kit may be in a pharmaceutical formulation and dosage suitable for a particular application and for a method of administration of the agents.

The kit may be designed to facilitate use of the methods described herein by physicians and can take many forms. Each of the compositions of the kit, where applicable, may be provided in liquid form (e.g., in solution), or in solid form, (e.g., a dry powder). In certain cases, some of the compositions may be constitutable or otherwise processable (e.g., to an active form), for example, by the addition of a suitable solvent or other species (for example, water or a cell culture medium), which may or may not be provided with the kit. As used herein, “instructions” can define a component of instruction and/or promotion, and typically involve written instructions on or associated with packaging of the invention. Instructions also can include any oral or electronic instructions provided in any manner such that a user will clearly recognize that the instructions are to be associated with the kit, for example, audiovisual (e.g., videotape, DVD, etc.), Internet, and/or web-based communications, etc. The written instructions may be in a form prescribed by a governmental agency regulating the manufacture, use or sale of pharmaceuticals or biological products, which instructions can also reflects approval by the agency of manufacture, use or sale for human administration.

The kit may contain any one or more of the components described herein in one or more containers. As an example, in one embodiment, the kit may include instructions for mixing one or more components of the kit and/or isolating and mixing a sample and applying to a subject. The kit may include a container housing agents described herein. The agents may be in the form of a liquid, gel or solid (powder). The agents may be prepared sterilely, to packaged in syringe and shipped refrigerated. Alternatively it may be housed in a vial or other container for storage. A second container may have other agents prepared sterilely. Alternatively the kit may include the active agents premixed and shipped in a syringe, vial, tube, or other container. The kit may have one or more or all of the components required to administer the agents to a patient, such as a syringe, topical application devices, or iv needle tubing and bag.

The kit may have a variety of forms, such as a blister pouch, a shrink wrapped pouch, a vacuum sealable pouch, a sealable thermoformed tray, or a similar pouch or tray form, with the accessories loosely packed within the pouch, one or more tubes, containers, a box or a bag. The kit may be sterilized after the accessories are added, thereby allowing the individual accessories in the container to be otherwise unwrapped. The kits can be sterilized using any appropriate sterilization techniques, such as radiation sterilization, heat sterilization, or other sterilization methods known in the art. The kit may also include other components, depending on the specific application, for example, containers, cell media, salts, buffers, reagents, syringes, needles, a fabric, such as gauze, for applying or removing a disinfecting agent, disposable gloves, a support for the agents prior to administration etc.

The present invention is further illustrated by the following Examples, which in no way should be construed as further limiting. The entire contents of all of the references (including literature references, issued patents, published patent applications, and co-pending patent applications) cited throughout this application are hereby expressly incorporated by reference, in particular for the teaching that is referenced hereinabove.

### Examples

Materials and Methods

Mice. CK-p25 double transgenic mice were raised on a doxycycline containing diet (at 1 mg/g) then switched to a normal diet at 6˜8 weeks of age to induce p25-GFP in a postnatal, forebrain-specific manner as described (Cruz et al., 2003). Individual mouse lines were backcrossed for multiple generations to obtain a homogeneous C57BL/6J background. Littermates and same sex mice were used for comparison whenever possible. All transgenes were heterozygous.

Microarray analyses. Total RNA was extracted from forebrains of 2 week induced CK-p25 Tg mice (n=3) and uninduced CK-p25 controls (n=3) using Trizol reagent (Sigma; St. Louis, Mo.). RNA was subjected to further purification with RNEasy columns (Qiagen; Hilden Germany), reverse transcribed, biotin-labeled, and hybridized onto Mouse Genome 430A 2.0 Arrays (Affymetrix, Santa Clara, Calif.) which represent approximately 14,000 well-characterized mouse genes. The set of genes differentially expressed at 2 weeks of induction was determined using dCHIP expression analyses software under the PM/MM difference model with standard parameters (Fold change threshold 1.2; lower 90% confidence bound of fold change). P values were <0.001 for clustering and median False Discovery rate was approximately 3.3%. To directly reference expression values for these genes at 8 weeks of induction, GeneChip Operating Software (GCOS, Affymetrix) was used to obtain absolute expression values for all experimental groups and to calculate fold change at 2 weeks, as shown in Table I. dCHIP expression values are shown in the Tables 2 and 3. Genes were grouped according to functional annotations from the Gene Ontology Database (http://www.geneontology.org/).

Comet assay. Primary rat cortical neurons at DIV 6˜8 were infected with herpesvirus expressing p25 (p25-HSV) or lacZ (lacZ-HSV). After 10 hours, neurons were dissociated and embedded in a thin layer of agarose. Lysis, alkaline treatment, and single cell gel electrophoreses (comet assay) was carried out as described with minor modifications (Dhawan et al., 2001).

Immunohistochemistry. Mice were perfused with 4% paraformaldehyde, brains were embedded in paraffin and sectioned, and subjected to citrate buffer based antigen retrieval and staining as described (Cruz et al., 2003). Antibodies to γH2AX (monoclonal from Upstate, Lake Placid, N.Y.; polyclonal from Trevigen, Gaithersburg, Md.), Ki-67 (Novocastra, Newcastle, Great Britain), PCNA (Oncogene Sciences, Cambridge, Mass.), phospho(pS10)-Histone H3(Upstate), and GFP (monoclonal from Santa Cruz, Santa Cruz, Calif.; polyclonal from Molecular Probes, Eugene, Oreg.) were used. While the CA1 region of hippocampus is shown in figures, similar results were observed in the cortex as well. Paraffin sections of human postmortem brains were subjected to antigen retrieval and stained with antibodies to γH2AX (Upstate) and HuD (Chemicon, Rosemont, Ill.). Ischemic rat brain sections were subjected to antigen retrieval and stained with antibody to γH2AX (Upstate).

Immunoblot Analysis. CK-p25 and control forebrains were dissected and homogenized in RIPA buffer (50 mM Tris, pH 8.0, 150 mM NaCl, 1% NP40, 0.5% sodium deoxycholate, 0.1% SDS) containing protease and phosphatase inhibitors. Equal quantities of brain lysates were subjected to SDS-PAGE and Western blot analysis using antibodies to γH2AX (Trevigen), alpha-tubulin (Sigma), E2F-1 (Santa Cruz), Cyclin A (Santa Cruz), p35 (Santa

Cruz), p27 (Santa Cruz), GFAP (Sigma), and BetaIII-tubulin (Sigma). Primary cultured rat or mouse cortical neurons at DIV 6˜8 were lysed in RIPA buffer plus SDS sample buffer (2% SDS, 0.6M DTT, 62.5 mM Tris, 10% glycerol). Equal quantities of lysate were subjected to SDS-PAGE and Western blot analysis using antibodies to γH2AX (Trevigen), p35 (Santa Cruz), alpha-tubulin (Sigma), B-galactosidase (Cortex Biochemicals, San Leandro, Calif.).

Luciferase Assays Hela cells were transfected with 200 ng reporter (containing E1b element and 5 Ga14 binding sites), 500 ng HDAC1-Ga14 fusion protein, and either 200 ng blank vector or 100 ng p25 plus 100 ng Cdk5 expression vectors, using Lipofectamine 2000 (Invitrogen,

Carlsbad, Calif.). At 15 hours post-transfection, cells were lysed with passive lysis buffer and luciferase assay was performed according to manufacturer's instructions (Promega, Madison, Wis.). Values were normalized to Ga14 protein levels as renilla reporters were also substantially repressed by HDAC1-Ga14.

Co-immunoprecipitation analyses. HEK293T cells were transfected with various constructs using Lipofectamine 2000. At 24 hours post-transfection, cells were lysed with IP buffer (0.4% Triton X-100, 200 mM NaCl, 50 mM Tris 7.5) containing protease and phosphatase inhibitors. Equal amounts of lysates were incubated with anti-flag-conjugated beads (Sigma) in IP buffer overnight, then washed three times in IP buffer Immune complexes were eluted by addition of sample buffer and boiling and analysed by SDS-PAGE. For in vivo analysis of p25/HDAC1 interaction, two week-induced CK-p25 mice and WT control forebrains were dounce homogenized in RIPA buffer and incubated with anti-HDAC1 (Abcam, Cambridge, Mass.) and protein sepharose G beads in a 1:4 dilution of RIPA:IP buffer overnight, washed three times in IP buffer, and eluted and analyzed by SDS-PAGE as described.

HDAC1 enzymatic activity assay. HEK293T cells were transfected with blank vector or with p25 and Cdk5 expression vectors with Lipofectamine 2000. Cells were lysed with IP buffer at 15 hours post-transfection, and immunoprecipitated with anti-HDAC1 (Abcam). Endogenous HDAC1 bound to beads were analyzed for histone deacetylase activity using the Histone deacetylase assay kit (Upstate) according to the manufacturer's instructions. Histone deacetylase activity was normalized to input HDAC1 protein levels which were analyzed by western blot. For analyses of HDAC1 activity in vivo, hippocampi were dissected from 2-week induced CK-p25 mice and WT littermates, and dounce homogenized in IP buffer with high salt (400mM NaC1) to aid HDAC1 extraction. Lysates were immunoprecipitated (in IP buffer with final 200 mM NaC1) and analyzed as described.

HDAC1 rescue assays. For cell death rescue assays, primary rat cortical neurons at DIV 5˜8 were transfected with p25-GFP plus blank vector or flag-HDAC1. At 24 hours post-transfection, neurons were fixed, stained, and GFP- and flag-positive neurons (for p25+HDAC1) and GFP positive neurons (for p25+vector) were scored based on nuclear morphology and neuritic integrity in a blind manner, as previously described (Konishi et al., 2002). It was noted that excessive levels of HDAC1 expression were neurotoxic (1 ug/well), and the neuroprotective effects of HDAC1 were observed at moderate levels of expression (250 ng/well). For γH2AX rescue assays, primary rat cortical neurons at DIV 5˜8 were transfected with flag-HDAC1, flag-HDAC2, or GFP and at 12 hours post-transfection, infected with p25-HSV at 85-90% infection rates. At 8 hours post-infection, cells were fixed and stained. Flag-(for HDAC1 or HDAC2) or GFP-positive neurons were scored for γH2AX immunoreactivity in a blind manner

Middle cerebral artery occlusion and transient forebrain ischemia. Adult Sprague-Dawley rats were subjected to one-hemisphere middle cerebral artery occlusion as previously described (Zhu et al., 2004). Three hours after filament withdrawal, mouse brains were fixed in 4% PFA, embedded in paraffin, and prepared as coronal sections. Infarct areas were identified by hematoxylin and eosin staining and adjacent sections were subjected to immunohistochemistry as described. For experiments examining HDAC1-mediated rescue of transient forebrain ischemia, rats were subjected to bilateral middle cerebral artery occlusion transient forebrain ischemia as described previously (Peng et al., 2006). Briefly, adult Sprague-Dawley rats were subjected to ischemia by bilaterally occluding common carotid arteries with aneurysm clips for 15 min, after which cerebral blood flow was restored. After 6 days, mice were processed and analyzed for Fluro-Jade staining and γH2AX staining using the previously described protocol (Wang et al., 2003). Briefly, after several washes in 0.01 M PBS, sections were incubated with blocking solution for 1 hr, followed by incubation with mono-clonal anti-gammaH2AX (1:200) at 4 C overnight. Sections were then incubated with anti-cy3 (1:200) for 1 hr. After being washed for 5 min in PBS and 5 min in distilled water, sections were then placed in 0.0001% Fluoro-Jade B staining solution with 0.1% acetic acid at 4 C for 1 hr. After 5 washes in distilled water for 5 min, sections were dried while covered. For histological quantification of neuronal death in striatal neurons, cells of interest were quantified from 30 μm thick coronal sections in an area of 0.26 mm² for each aspect of the striatum (dorsal striatum, dorsal lateral, ventral-medial and ventral-lateral). Coronal sections showing the striatum, e.g. rostrocaudal levels plus 1 mm, were scanned with a 20 X imaging microscope motorized for X, Y and Z displacements using the imaging acquisition and analysis system. Analyzed areas in the striatum encompassed the entire striatal region. This represented, on average, 300-500 contiguous digitized images per animal, corresponding o contiguous 112×91 um field of view. Image pixels were 0.12×0.12 um in size. Each field of view was acquired at 12 equidistant different focal planes over 5 um along the z-axis within the section. Averaged neuronal cell counts were obtained from six animals per group.

Chromatin Fractionation. Chromatin fractionation was based on a previous protocol (Andegeko et al., 2001). Rat primary neurons at DIV5-7 were infected with GFP-HSV or p25GFP-HSV. At 20 hours later, cells were washed, scraped in hypotonic buffer plus protease and phosphatase inhibitor, and subjected to hypotonic lysis aided by 10 passages through a 19G syringe. Cells were spun down for 5 minutes at 1000 g, and the supernatant was collected as the cytosolic fraction. The pellet was washed once in hypotonic buffer then resuspended in 0.5% NP-40 buffer (0.5% NP-40, 50 mM Hepes pH 7.5, 150 mM NaCl, 1 mM EDTA, protease and phosphatase inhibitors) and incubated on ice for 40 minutes with occasional pipetting. Samples were then centrifuged for 15 minutes at 16000 g. Supernatant was collected as the non-chromatin bound nuclear fraction. The pellet was washed once in 0.5% NP-40 buffer, then extracted by addition of SDS loading buffer and boiling. This final fraction contains chromatin-bound proteins and insoluble proteins (Andegeko et al., 2001).

Chromatin Immunoprecipitation. For chromatin immunoprecipitation experiments, 293T cells were transfected with the indicated constructs, fixed 14 hours after transfection with 1% formaldehyde, and processed according to manufacturer's instructions (#17-195, Upstate). Monoclonal HDAC1 (ChIP grade, Abcam) was used to immunoprecipitate endogenous HDAC1. The following sequences were used to amplify core promoter regions:

(SEQ ID NO: 1)

p21 (Forward: 5′-GGT GTC TAG GTG CTC CAG GT-3′,

(SEQ ID NO: 2)

Reverse: 5′-GCA CTC TCC AGG AGG ACA CA-3′

(SEQ ID NO: 3)

E2F-1 (Forward: 5′-CAC ACC GCG CCT GGT ACC-3′,

(SEQ ID NO: 5)

Reverse: 5′-CCG CTG CCT GCA AAG TCC-3′.

Fear conditioning. Fear conditioning experiments were carried out as previously described (Kim et al., 2007), using a fear conditioning apparatus (TSE Systems, Midland, Mich.).

HDAC inhibitors. SAHA (Breslow et al. 1993) and MS-275 (Susuki et al. 2001) were synthesized following published procedures outlined in the following references: Breslow, R, Marks, P A., Rifkind, R A., Jursic, B. Novel potent inducers of terminal differentiation and methods thereof. PTC Int. Appl. WO93/07148, Apr. 15, 1993; Suzuki, T., Tomoyuki, A., Tsuchiya, K., Ishibashi, H. Method of producing benzamide derivatives. U.S. Pat. No. 6,320,078, Nov. 20, 2001.

### Experiment 1: Gene Expression Profile of CK-p25 Transgenic Mice

We carried out microarray analyses (Affymetrix) on CK-p25 mice induced for only 2 weeks, when no signs of neurotoxicity or reactive astrogliosis are present, to elucidate the initiating mechanisms which may account for the neurodegeneration seen later. A total of 225 genes (292 total probes) were found to be significantly altered in the induced transgenics compared to uninduced controls (Table 2). Surprisingly, genes involved in cell cycle or DNA damage repair/response (Gene Ontology database, http://www.geneontology.org/) were highly represented (Table 3), totaling 65 genes (84 total probes) with significant overlap to between the annotation groups. Representative genes from these groups are summarized in Table 1. 63 of the 65 genes were upregulated, including cell cycle/proliferation genes such as Cyclins A, B, and E, E2F-1, Ki67 and PCNA, which have previously been shown to be upregulated in postmortem AD brains and rodent stroke models. In addition, a number of DNA damage response genes, in particular genes involved in the DNA double strand breaks response such as Rad51, BRCA1, and Checkpoint 1, were found to be highly upregulated. Collectively, these findings suggest the aberrant expression of cell cycle proteins and a response to double strand DNA breaks in the brains of CK-p25 mice.

TABLE 1
Summary of specific cell cycle related and
DNA damage responsive genes.

| Function | Gene name | Accession | Fold (Δ) |
|---|---|---|---|
| Cell cycle related | Cyclin A2 | X75483 | 3.78 |
| genes | Cyclin B1 | NM_007629 | 10.58 |
| | Cyclin E1 | NM_007633 | 1.94 |
| | Cyclin E2 | AF091432 | 4.9 |
| | Cdc28 | NM_016904 | 2.4 |
| | Cdc28 regulatory subunit 1 | NM_025415 | 5.11 |
| | Cdc2a (cdk1) | NM_007659 | 8.47 |
| | Cdc20 | BB041150 | 4.09 |
| | Cell division associated 1 | AK010351 | 2.16 |
| | (Nuf2R) | | |
| | Polo-like kinase 4 | AI385771 | 2.9 |
| | Geminin | NM_020567 | 2.27 |
| | Mcm2 | NM_008564 | 5.01 |
| | Mcm3 | C80350 | 6.27 |
| | Mcm4 | BC013094 | 3.06 |
| | Mcm6 | NM_008567 | 6.04 |
| | Mcm7 | NM_008568 | 3.78 |
| | DNA primase, p49 subunit | J04620 | 3.19 |
| | DNA primase, p58 subunit | NM_008922 | 1.67 |
| | p21/WAF | AK007630 | 2.55 |
| | Proliferating cell nuclear | BC010343 | 2.47 |
| | antigen (PCNA) | | |
| | Ki-67 proferation antigen | X82786 | 16.14 |
| | E2F-1 | NM_007891 | 5.04 |
| | Transcription factor DP-1 | BG075396 | 1.73 |
| DNA damage | Rad51 | NM_011234 | 31.77 |
| responsive genes | Rad51 associated protein | BC003738 | 9.93 |
| | Topoisomerase II alpha | BM211413 | 6.02 |
| | DNA methyltransferase | NM_010066 | 1.67 |
| | (cytosine-5) 1 | | |
| | Flap endonuclease | BB393998 | 2.65 |
| | MutS homolog 6 | U42190 | 1.66 |
| | Ligase I | NM_010715 | 3.99 |
| | DNA polymerase epsilon | NM_011132 | 37.8 |
| | DNA polymerase delta 1, | BB385244 | 1.93 |
| | catalytic subunit | | |
| | Pmaip1 | NM_021451 | 5.23 |
| | Deoxyuridine triphosphatase | AF091101 | 1.65 |
| | Ribonucleotide reductase M2 | BB758819 | 5.65 |
| | Replication protein A1 | BB491281 | 1.64 |
| | Replication protein A2 | AK011530 | 2.13 |
| | Uracil DNA glycosylase | BC004037 | 3.24 |
| | Chromatin assembly factor 1b | NM_011132 | 5.81 |
| | BRCA1 | U31625 | 5.45 |
| | Checkpoint 1 | C85740 | 8.06 |
| | Mad2-like 1 | NM_019499 | 2.59 |

TABLE 2
Complete list of genes with altered expression in 2 week induced CK-p25 mice compared to
uninduced controls.

| | | | baseline | baseline | exp | exp | FOLD |
|---|---|---|---|---|---|---|---|
| probe set | gene | Accession | mean | mean SE | mean | mean SE | CHANGE |
| 1415810_at | ubiquitin-like, containing PHD | BB702754 | 8.85 | 5.91 | 204.31 | 25.12 | 23.08 |
| | and RING finger domains, 1 | | | | | | |
| 1415829_at | lamin B receptor | NM_133815 | 308.65 | 13.26 | 425.65 | 22.39 | 1.38 |
| 1415878_at | ribonucleotide reductase M1 | BB758819 | 117.53 | 21.29 | 362.03 | 36.02 | 3.08 |
| 1415899_at | Jun-B oncogene | NM_008416 | 1458.85 | 63.86 | 1007.27 | 55.48 | −1.45 |
| 1415945_at | minichromosome | NM_008566 | 82.62 | 6.79 | 598.4 | 40.58 | 7.24 |
| | maintenance deficient 5, cell | | | | | | |
| | division cycle 46 (S. cerevisiae) | | | | | | |
| 1416030_a_at | minichromosome | NM_008568 | 127.89 | 12.51 | 540.55 | 54.49 | 4.23 |
| | maintenance deficient 7 (S. cerevisiae) | | | | | | |
| 1416031_s_at | minichromosome | NM_008568 | 104.16 | 16.5 | 404.63 | 38.45 | 3.88 |
| | maintenance deficient 7 (S. cerevisiae) | | | | | | |
| 1416042_s_at | nuclear autoantigenic sperm | BB493242 | 209.13 | 14.65 | 430.55 | 40.63 | 2.06 |
| | protein (histone-binding) | | | | | | |
| 1416066_at | CD9 antigen | NM_007657 | 790.37 | 37.64 | 1178.72 | 41.07 | 1.49 |
| 1416214_at | minichromosome | BC013094 | 137.85 | 13.4 | 447.68 | 30.03 | 3.25 |
| | maintenance deficient 4 | | | | | | |
| | homolog (S. cerevisiae) | | | | | | |
| 1416251_at | minichromosome | NM_008567 | 167.95 | 26.1 | 1346.48 | 120.1 | 8.02 |
| | maintenance deficient 6 | | | | | | |
| | (MIS5 homolog, S. pombe) | | | | | | |
| | (S. cerevisiae) | | | | | | |
| 1416287_at | regulator of G-protein | NM_009062 | 1125.85 | 26.02 | 804.57 | 33.27 | −1.4 |
| | signaling 4 | | | | | | |
| 1416382_at | cathepsin C | NM_009982 | 174.48 | 12.38 | 377.42 | 54.15 | 2.16 |
| 1416433_at | replication protein A2 | BC004578 | 154.93 | 15.9 | 345.73 | 29.37 | 2.23 |
| 1416492_at | cyclin E1 | NM_007633 | 124.08 | 12.81 | 253.43 | 19.98 | 2.04 |
| 1416505_at | nuclear receptor subfamily 4, | NM_010444 | 1733.42 | 123.96 | 1195.21 | 101.94 | −1.45 |
| | group A, member 1 | | | | | | |
| 1416575_at | cell division cycle 45 homolog | NM_009862 | 24.19 | 5.77 | 151.32 | 16.09 | 6.26 |
| | (S. cerevisiae)-like | | | | | | |
| 1416641_at | ligase I, DNA, ATP-dependent | NM_010715 | 133.63 | 14.08 | 535.21 | 55.86 | 4.01 |
| 1416698_a_at | CDC28 protein kinase 1 | NM_016904 | 145 | 20.37 | 393.4 | 22.1 | 2.71 |
| 1416773_at | wee 1 homolog (S. pombe) | NM_009516 | 358.95 | 18.28 | 494.28 | 21.48 | 1.38 |
| 1416915_at | mutS homolog 6 (E. coli) | U42190 | 197.78 | 9.83 | 318.35 | 12.04 | 1.61 |
| 1416926_at | transformation related protein | AW495711 | 368.88 | 12.21 | 516.09 | 38.61 | 1.4 |
| | 53 inducible nuclear protein 1 | | | | | | |
| 1417063_at | complement component 1, q | NM_009777 | 933.13 | 52.8 | 1762.24 | 209.18 | 1.89 |
| | subcomponent, beta | | | | | | |
| | polypeptide | | | | | | |
| 1417139_at | RIKEN cDNA 1700022L09 | NM_025853 | 60.83 | 8.87 | 177.09 | 19.8 | 2.91 |
| | gene | | | | | | |
| 1417141_at | interferon gamma induced | NM_018738 | 187.4 | 18.51 | 457.49 | 77.71 | 2.44 |
| | GTPase | | | | | | |
| 1417244_a_at | interferon regulatory factor 7 | NM_016850 | 87.6 | 13.65 | 254.61 | 51.5 | 2.91 |
| 1417266_at | chemokine (C-C motif) ligand 6 | BC002073 | 51.24 | 9.74 | 167.31 | 29.76 | 3.27 |
| 1417323_at | RIKEN cDNA 5430413I02 | NM_019976 | 126.6 | 12.78 | 417.22 | 62.52 | 3.3 |
| | gene | | | | | | |
| 1417381 _at | complement component 1, q | NM_007572 | 1458.29 | 82.19 | 2642.53 | 284.95 | 1.81 |
| | subcomponent, alpha | | | | | | |
| | polypeptide | | | | | | |
| 1417457_at | CDC28 protein kinase | NM_025415 | 54.44 | 8.79 | 365.98 | 40.38 | 6.72 |
| | regulatory subunit 2 | | | | | | |
| 1417458_s_at | CDC28 protein kinase | NM_025415 | 76.69 | 6.74 | 443.96 | 37.63 | 5.79 |
| | regulatory subunit 2 | | | | | | |
| 1417503_at | replication factor C (activator | NM_020022 | 520.32 | 26.11 | 737.8 | 35.95 | 1.42 |
| | 1) 2 | | | | | | |
| 1417506_at | geminin | NM_020567 | 121.76 | 17.02 | 309.1 | 16.65 | 2.54 |
| 1417541_at | helicase, lymphoid specific | NM_008234 | 16.62 | 3.97 | 179.78 | 25.82 | 10.82 |
| 1417586_at | timeless homolog | BM230269 | 47.47 | 8.51 | 261.15 | 18.18 | 5.5 |
| | (Drosophila) | | | | | | |
| 1417822_at | DNA segment, Chr 17, human | NM_033075 | 48.68 | 7.55 | 195.12 | 14.95 | 4.01 |
| | D6S56E 5 | | | | | | |
| 1417830_at | SMC (structural maintenance | BB156359 | 650.81 | 36.6 | 881.88 | 37.39 | 1.36 |
| | of chromosomes 1)-like 1 (S. cerevisiae) | | | | | | |
| 1417868_a_at | cathepsin Z | NM_022325 | 770.58 | 49.38 | 1458.02 | 122.33 | 1.89 |
| 1417869_s_at | cathepsin Z | NM_022325 | 328.19 | 28.57 | 638.98 | 49.39 | 1.95 |
| 1417870_x_at | cathepsin Z | NM_022325 | 635.33 | 48.16 | 1241.39 | 97.29 | 1.95 |
| 1417878_at | E2F transcription factor 1 | NM_007891 | 65.86 | 15.12 | 360.15 | 28.76 | 5.47 |
| 1417910_at | cyclin A2 | X75483 | 45.28 | 10.2 | 342.45 | 34.42 | 7.56 |
| 1417926_at | RIKEN cDNA 5830426I05 | NM_133762 | 50.46 | 5.52 | 180.7 | 16.75 | 3.58 |
| | gene | | | | | | |
| 1417938_at | RAD51 associated protein 1 | BC003738 | 26.57 | 5.13 | 359.98 | 46.41 | 13.55 |
| 1417947_at | proliferating cell nuclear | BC010343 | 1269.31 | 98.2 | 3142.66 | 162.7 | 2.48 |
| | antigen | | | | | | |
| 1417961 _a_at | tripartite motif protein 30 | BM240719 | 48.69 | 7.78 | 190.17 | 44.39 | 3.91 |
| 1418021_at | complement component 4 | NM_009780 | 261.38 | 24.3 | 532.63 | 70.51 | 2.04 |
| | (within H-2S) | | | | | | |
| 1418036_at | DNA primase, p58 subunit | NM_008922 | 110.45 | 13.1 | 223.34 | 15.06 | 2.02 |
| 1418051_at | Eph receptor B6 | NM_007680 | 405.82 | 12.25 | 304.15 | 13.07 | −1.33 |
| 1418090_at | plasmalemma vesicle | NM_032398 | 125.6 | 10.88 | 263.34 | 14.5 | 2.1 |
| | associated protein | | | | | | |
| 1418161_at | junctophilin 3 | NM_020605 | 1557.21 | 32.54 | 1148.4 | 69.22 | −1.36 |
| 1418191_at | ubiquitin specific protease 18 | NM_011909 | 29.81 | 5.16 | 222.57 | 52.23 | 7.47 |
| 1418203_at | phorbol-12-myristate-13- | NM_021451 | 37.55 | 10.11 | 278.08 | 46.85 | 7.4 |
| | acetate-induced protein 1 | | | | | | |
| 1418204_s_at | allograft inflammatory factor 1 | NM_019467 | 128.48 | 21.58 | 272.5 | 21.16 | 2.12 |
| 1418240_at | guanylate nucleotide binding | NM_010260 | 53.15 | 8.42 | 195.47 | 41.99 | 3.68 |
| | protein 2 | | | | | | |
| 1418264_at | SoxLZ/Sox6 leucine zipper | NM_021790 | 37.43 | 11.05 | 282.88 | 26.43 | 7.56 |
| | binding protein in testis | | | | | | |
| 1418281_at | RAD51 homolog (S. cerevisiae) | NM_011234 | 1.82 | 9.76 | 386.38 | 54.82 | 212.33 |
| 1418293_at | interferon-induced protein with | NM_008332 | 124.08 | 8.63 | 413.76 | 33.72 | 3.33 |
| | tetratricopeptide repeats 2 | | | | | | |
| 1418340_at | Fc receptor, IgE, high affinity | NM_010185 | 279.37 | 27.39 | 525.25 | 47.94 | 1.88 |
| | I, gamma polypeptide | | | | | | |
| 1418365_at | cathepsin H | NM_007801 | 291.83 | 6.96 | 449.72 | 32.21 | 1.54 |
| 1418369_at | DNA primase, p49 subunit | J04620 | 151.7 | 12.93 | 416.33 | 24.11 | 2.74 |
| 1418392_a_at | guanylate nucleotide binding | NM_018734 | 93.66 | 14.38 | 355.67 | 92.77 | 3.8 |
| | protein 3 | | | | | | |
| 1418580_at | RIKEN cDNA 5830458K16 | BC024872 | 83.18 | 9.92 | 449.05 | 99.24 | 5.4 |
| | gene | | | | | | |
| 1418687_at | activity regulated cytoskeletal- | NM_018790 | 1559.41 | 154.11 | 777.39 | 65.75 | −2.01 |
| | associated protein | | | | | | |
| 1418825_at | interferon inducible protein 1 | NM_008326 | 147.29 | 13.21 | 424.05 | 43.11 | 2.88 |
| 1418930_at | chemokine (C—X—C motif) | NM_021274 | 12.02 | 8.54 | 828.36 | 237.55 | 68.89 |
| | ligand 10 | | | | | | |
| 1419042_at | expressed sequence | BM239828 | 38.23 | 8.08 | 250.13 | 64.02 | 6.54 |
| | AW111922 | | | | | | |
| 1419043_a_at | expressed sequence | BM239828 | 46.51 | 9.44 | 251.21 | 67.88 | 5.4 |
| | AW111922 | | | | | | |
| 1419100_at | serine (or cysteine) proteinase | NM_009252 | 160.25 | 21.61 | 347.56 | 32.65 | 2.17 |
| | inhibitor, clade A, member 3N | | | | | | |
| 1419153_at | RIKEN cDNA 2810417H13 | AK017673 | 44.52 | 14.36 | 314.67 | 37.45 | 7.07 |
| | gene | | | | | | |
| 1419202_at | cystatin F (leukocystatin) | NM_009977 | 4.57 | 6.61 | 140.95 | 35.93 | 30.85 |
| 1419224_at | cat eye syndrome | NM_033567 | 418.92 | 40.49 | 277.23 | 12.66 | −1.51 |
| | chromosome region, | | | | | | |
| | candidate 6 homolog (human) | | | | | | |
| 1419270_a_at | deoxyuridine triphosphatase | AF091101 | 479.7 | 42.4 | 794.29 | 51.19 | 1.66 |
| 1419282_at | chemokine (C-C motif) ligand | U50712 | 27.09 | 8.38 | 254.1 | 47.62 | 9.38 |
| | 12 | | | | | | |
| 1419414_at | guanine nucleotide binding | AB030194 | 1445.68 | 39.61 | 999.94 | 81.29 | −1.45 |
| | protein 13, gamma | | | | | | |
| 1419569_a_at | interferon-stimulated protein | BC022751 | 33.41 | 6.8 | 148.21 | 27.01 | 4.44 |
| 1419835_s_at | plectin 1 | AW123286 | 1090.1 | 27.12 | 824.92 | 32.02 | −1.32 |
| 1419838_s_at | polo-like kinase 4 (Drosophila) | AI385771 | 62.68 | 9.98 | 186.45 | 11.33 | 2.97 |
| 1419943_s_at | cyclin B1 | AU015121 | 21.93 | 7.08 | 134.26 | 22.88 | 6.12 |
| 1419978_s_at | DNA segment, Chr 10, | AU014694 | 1468.35 | 31.2 | 1158.18 | 28.41 | −1.27 |
| | ERATO Doi 610, expressed | | | | | | |
| 1420028_s_at | minichromosome | C80350 | 35.85 | 7.21 | 386.12 | 33 | 10.77 |
| | maintenance deficient 3 (S. cerevisiae) | | | | | | |
| 1420699_at | C-type (calcium dependent, | NM_020008 | 7.32 | 9.22 | 108.17 | 22.24 | 14.78 |
| | carbohydrate recognition | | | | | | |
| | domain) lectin, superfamily | | | | | | |
| | member 12 | | | | | | |
| 1420915_at | signal transducer and | AW214029 | 129.87 | 7.21 | 305.95 | 42.94 | 2.36 |
| | activator of transcription 1 | | | | | | |
| 1421015_s_at | polymerase (DNA directed), | NM_021498 | 186.87 | 19.92 | 287.66 | 11.06 | 1.54 |
| | epsilon 3 (p17 subunit) | | | | | | |
| 1421217_a_at | lectin, galactose binding, | NM_010708 | 145.17 | 19.82 | 364.33 | 59.79 | 2.51 |
| | soluble 9 | | | | | | |
| 1421322_a_at | interferon dependent positive | NM_008394 | 79.34 | 10.18 | 186.9 | 31.27 | 2.36 |
| | acting transcription factor 3 | | | | | | |
| | gamma | | | | | | |
| 1421446_at | protein kinase C, gamma | NM_011102 | 803.69 | 52.13 | 507.25 | 25.87 | −1.58 |
| 1421546_a_at | Rac GTPase-activating | NM_012025 | 74.74 | 10.62 | 246.04 | 25.68 | 3.29 |
| | protein 1 | | | | | | |
| 1421731 _a_at | flap structure specific | NM_007999 | 137.71 | 20.22 | 351.18 | 27.44 | 2.55 |
| | endonuclease 1 | | | | | | |
| 1421739_a_at | megakaryocyte-associated | NM_010768 | 1050.22 | 27.87 | 808.35 | 33.29 | −1.3 |
| | tyrosine kinase | | | | | | |
| 1421792_s_at | triggering receptor expressed | NM_031254 | 71.19 | 20.71 | 191.25 | 26 | 2.69 |
| | on myeloid cells 2 | | | | | | |
| 1421840_at | ATP-binding cassette, sub- | BB144704 | 632.11 | 38.85 | 906.2 | 74.23 | 1.43 |
| | family A (ABC1), member 1 | | | | | | |
| 1422016_a_at | centromere autoantigen H | BC025084 | 11.16 | 4.61 | 160.93 | 18.52 | 14.42 |
| 1422430_at | fidgetin-like 1 | NM_021891 | 64.89 | 8.71 | 242.4 | 9.67 | 3.74 |
| 1422460_at | MAD2 (mitotic arrest deficient, | NM_019499 | 147.87 | 16.85 | 323.31 | 12.94 | 2.19 |
| | homolog)-like 1 (yeast) | | | | | | |
| 1422535_at | cyclin E2 | AF091432 | 108.4 | 21.69 | 507.25 | 53.46 | 4.68 |
| 1422609_at | cAMP-regulated | BE648432 | 2531.98 | 64.67 | 1973.5 | 59.49 | −1.28 |
| | phosphoprotein 19 | | | | | | |
| 1422903_at | lymphocyte antigen 86 | NM_010745 | 438.62 | 35.17 | 1141.01 | 188.6 | 2.6 |
| 1422946_a_at | DNA methyltransferase | NM_010066 | 413.52 | 18.37 | 741.17 | 39.72 | 1.79 |
| | (cytosine-5) 1 | | | | | | |
| 1422948_s_at | histone 1, H4h | NM_013550 | 220.58 | 9.6 | 362.74 | 46.9 | 1.64 |
| 1423100_at | FBJ osteosarcoma oncogene | AV026617 | 1512.22 | 109.28 | 1041.52 | 46.33 | −1.45 |
| 1423241_a_at | transcription factor Dp 1 | BG075396 | 480.48 | 20.48 | 747.43 | 58.29 | 1.56 |
| 1423293_at | replication protein A1 | BM244983 | 517.64 | 29.86 | 845.63 | 39.32 | 1.63 |
| 1423371_at | polymerase (DNA-directed), | BF577544 | 333.08 | 16.92 | 524.1 | 30.16 | 1.57 |
| | epsilon 4 (p12 subunit) | | | | | | |
| 1423372_at | polymerase (DNA-directed), | BF577544 | 446.02 | 31.89 | 615.01 | 20.41 | 1.38 |
| | epsilon 4 (p12 subunit) | | | | | | |
| 1423440_at | RIKEN cDNA 1110001A07 | AK003196 | 181.02 | 16.26 | 328.63 | 29.66 | 1.82 |
| | gene | | | | | | |
| 1423514_at | glucokinase activity, related | AI449806 | 135.66 | 11.38 | 237.06 | 8.8 | 1.75 |
| | sequence 1 | | | | | | |
| 1423565_at | phosphoribosylaminoimidazole | BM207712 | 1296.46 | 31.59 | 1634.93 | 27.68 | 1.26 |
| | carboxylase, | | | | | | |
| | phosphoribosylaminoribosyla | | | | | | |
| | minoimidazole, | | | | | | |
| | succinocarboxamide | | | | | | |
| | synthetase | | | | | | |
| 1423643_at | DEAD (Asp-Glu-Ala-Asp) box | BC020134 | 182.66 | 6.11 | 308.91 | 18.49 | 1.69 |
| | polypeptide 39 | | | | | | |
| 1423674_at | ubiquitin specific protease 1 | BC018179 | 102.77 | 7.19 | 207.75 | 15.67 | 2.02 |
| 1423714_at | ASF1 anti-silencing function 1 | BC003428 | 31.92 | 11.86 | 160.19 | 15.08 | 5.02 |
| | homolog B (S. cerevisiae) | | | | | | |
| 1423754_at | interferon induced | BC010291 | 749.75 | 93.86 | 1755.02 | 315.88 | 2.34 |
| | transmembrane protein 3 | | | | | | |
| 1423809_at | transcription factor 19 | BC004617 | 115.16 | 12.31 | 746.04 | 76.27 | 6.48 |
| 1423847_at | RIKEN cDNA 2810406C15 | BC025460 | 192.64 | 7.96 | 339.89 | 15.8 | 1.76 |
| | gene | | | | | | |
| 1423947_at | RIKEN cDNA 1110008P14 | BC024615 | 1534.55 | 46.65 | 1067.46 | 24.94 | −1.44 |
| | gene | | | | | | |
| 1424078_s_at | peroxisomal biogenesis factor 6 | BC003424 | 426.36 | 12.33 | 316.27 | 11.2 | −1.35 |
| 1424118_a_at | RIKEN cDNA 2600017H08 | BC027121 | 23.1 | 7.81 | 614.19 | 92.82 | 26.59 |
| | gene | | | | | | |
| 1424143_a_at | retroviral integration site 2 | AF477481 | 57.02 | 13.4 | 931.34 | 59.35 | 16.33 |
| 1424144_at | retroviral integration site 2 | AF477481 | 22.25 | 14.75 | 489.72 | 40.35 | 22.01 |
| 1424278_a_at | baculoviral IAP repeat- | BC004702 | 20.81 | 4.41 | 172.03 | 11.66 | 8.26 |
| | containing 5 | | | | | | |
| 1424321_at | replication factor C (activator | BC003335 | 113.26 | 14.16 | 305.38 | 18.87 | 2.7 |
| | 1) 4 | | | | | | |
| 1424629_at | breast cancer 1 | U31625 | 31.3 | 8.35 | 150.19 | 17.3 | 4.8 |
| 1424638_at | cyclin-dependent kinase | AK007630 | 254.59 | 49.08 | 574.02 | 126.42 | 2.25 |
| | inhibitor 1A (P21) | | | | | | |
| 1424674_at | solute carrier family 39 (metal | BB825002 | 620.71 | 36.28 | 830.13 | 19.83 | 1.34 |
| | ion transporter), member 6 | | | | | | |
| 1424921_at | RIKEN cDNA 2310015I10 | BC008532 | 71.46 | 10.27 | 225.78 | 35.85 | 3.16 |
| | gene | | | | | | |
| 1424948_x_at | histocompatibility 2, K1, K | L23495 | 205.89 | 23.24 | 394.36 | 60.2 | 1.92 |
| | region | | | | | | |
| 1425271_at | proteasome (prosome, | AB000121 | 83.85 | 12.61 | 187.73 | 9.83 | 2.24 |
| | macropain) 26S subunit, | | | | | | |
| | ATPase 3, interacting protein | | | | | | |
| 1425336_x_at | histocompatibility 2, K1, K | BC011306 | 530.22 | 52.89 | 983.65 | 141.11 | 1.86 |
| | region | | | | | | |
| 1425382_a_at | aquaporin 4 | U48399 | 511.65 | 63.16 | 805.49 | 59.8 | 1.57 |
| 1425545_x_at | histocompatibility 2, K1, K | M86502 | 654.47 | 41.11 | 1153.78 | 174.66 | 1.76 |
| | region | | | | | | |
| 1425753_a_at | uracil-DNA glycosylase | BC004037 | 32.27 | 6.23 | 146.89 | 9.68 | 4.55 |
| | hyaluronan mediated motility | | | | | | |
| 1425815_a_at | receptor (RHAMM) | BC021427 | 86.73 | 8.55 | 197.23 | 24.38 | 2.27 |
| 1426278_at | RIKEN cDNA 2310061N23 | AY090098 | 70.7 | 18.86 | 422.13 | 92.35 | 5.97 |
| | gene | | | | | | |
| 1426473_at | DnaJ (Hsp40) homolog, | BM942465 | 515.38 | 17.28 | 1068.82 | 77.21 | 2.07 |
| | subfamily C, member 9 | | | | | | |
| 1426508_at | glial fibrillary acidic protein | BB183081 | 1103.93 | 55.6 | 2458.65 | 365.61 | 2.23 |
| 1426509_s_at | glial fibrillary acidic protein | BB183081 | 1153.11 | 65.26 | 2386.03 | 373.67 | 2.07 |
| 1426612_at | timeless interacting protein | AK011357 | 263.63 | 53.25 | 528.06 | 29.96 | 2 |
| 1426652_at | minichromosome | BI658327 | 14.92 | 7.31 | 191.36 | 8.4 | 12.82 |
| | maintenance deficient 3 (S. cerevisiae) | | | | | | |
| 1426653_at | minichromosome | BI658327 | 63.81 | 18.71 | 198.02 | 8.17 | 3.1 |
| | maintenance deficient 3 (S. cerevisiae) | | | | | | |
| 1426729_at | RIKEN cDNA 2900046G09 | BC003957 | 816.11 | 27.71 | 571.5 | 43.2 | −1.43 |
| | gene | | | | | | |
| 1426738_at | diacylglycerol kinase zeta | BC014860 | 1236.02 | 90.53 | 875.63 | 48.37 | −1.41 |
| 1426739_at | downstream neighbor of SON | BQ174742 | 197.57 | 26.85 | 349.48 | 45.13 | 1.77 |
| 1426788_a_at | structure specific recognition | BC024835 | 790.83 | 16.29 | 1044.88 | 48.87 | 1.32 |
| | protein 1 | | | | | | |
| 1426790_at | structure specific recognition | BC024835 | 499.56 | 13.91 | 687.06 | 30.92 | 1.38 |
| | protein 1 | | | | | | |
| 1426817_at | antigen identified by | X82786 | 28.14 | 9.18 | 245.79 | 41.74 | 8.74 |
| | monoclonal antibody Ki 67 | | | | | | |
| 1426838_at | polymerase (DNA-directed), | AK010805 | 211.19 | 23.1 | 402.22 | 21.31 | 1.9 |
| | delta 3, accessory subunit | | | | | | |
| 1426855_at | DNA segment, Chr 10, | AK010452 | 591.71 | 13.57 | 453.87 | 13.45 | −1.3 |
| | ERATO Doi 610, expressed | | | | | | |
| 1427076_at | macrophage expressed gene 1 | L20315 | 185.64 | 22.01 | 571.97 | 80.35 | 3.08 |
| 1427105_at | RIKEN cDNA 2610510J17 | BM230253 | 56.66 | 12.15 | 180.13 | 29.34 | 3.18 |
| | gene | | | | | | |
| 1427275_at | SMC4 structural maintenance | BI665568 | 159.11 | 13.89 | 607.23 | 74.11 | 3.82 |
| | of chromosomes 4-like 1 | | | | | | |
| | (yeast) | | | | | | |
| 1427541_x_at | hyaluronan mediated motility | X64550 | 12.62 | 5.29 | 116.16 | 16.3 | 9.2 |
| | receptor (RHAMM) | | | | | | |
| 1427724_at | topoisomerase (DNA) II alpha | U01919 | 47.19 | 16.53 | 147.38 | 22.73 | 3.12 |
| 1427746_x_at | histocompatibility 2, K1, K | S70184 | 230.54 | 16.76 | 427.79 | 77.63 | 1.86 |
| | region | | | | | | |
| 1428061_at | histidine aminotransferase 1 | AK014330 | 248.97 | 17.5 | 475.73 | 27.38 | 1.91 |
| 1428114_at | solute carrier family 14 (urea | AW556396 | 155.67 | 19.62 | 256.98 | 23.86 | 1.65 |
| | transprorter), member 1 | | | | | | |
| 1428531_at | RIKEN cDNA 5930412E23 | BB457797 | 345.55 | 12.15 | 477.92 | 20.56 | 1.38 |
| | gene | | | | | | |
| 1428639_at | RIKEN cDNA 2700022J23 | AK012271 | 102.81 | 3.59 | 267.45 | 22.05 | 2.6 |
| | gene | | | | | | |
| 1429270_a_at | RIKEN cDNA 1700013H19 | AK005954 | 87.4 | 10.72 | 366.06 | 33.25 | 4.19 |
| | gene | | | | | | |
| 1429491_s_at | DNA segment, Chr 2, ERATO | AK018316 | 322.17 | 24.91 | 477.99 | 36.02 | 1.48 |
| | Doi 145, expressed | | | | | | |
| 1430811_a_at | cell division cycle associated 1 | AK010351 | 91.63 | 13.29 | 207.53 | 18.55 | 2.26 |
| 1431591_s_at | interferon, alpha-inducible | AK019325 | 50.91 | 8.93 | 330.95 | 91.32 | 6.5 |
| | protein | | | | | | |
| 1431946_a_at | amyloid beta (A4) precursor | AK013520 | 356.8 | 9.55 | 242.52 | 15.6 | −1.47 |
| | protein-binding, family A, | | | | | | |
| | member 1 binding protein | | | | | | |
| 1433674_a_at | RNA, U22 small nucleolar | BQ177137 | 200.08 | 22.2 | 450.98 | 13.08 | 2.25 |
| 1433675_at | RNA, U22 small nucleolar | BQ177137 | 159.2 | 19.83 | 380.35 | 32.55 | 2.39 |
| 1433685_a_at | RIKEN cDNA 6430706D22 | BM248225 | 206.82 | 19.04 | 405.85 | 68.43 | 1.96 |
| | gene | | | | | | |
| 1433954_at | RIKEN cDNA 4632419I22 | AV227569 | 127.54 | 9.03 | 268.64 | 16.84 | 2.11 |
| | gene | | | | | | |
| 1434079_s_at | minichromosome | BB699415 | 87.64 | 12.77 | 432.69 | 16.22 | 4.94 |
| 1434299_x_at | maintenance deficient 2 | AI413098 | 754.44 | 39.13 | 1005.03 | 35.2 | 1.33 |
| | mitotin (S. cerevisiae) | | | | | | |
| | RAB, member of RAS | | | | | | |
| | oncogene family-like 4 | | | | | | |
| 1434366_x_at | complement component 1, q | AW227993 | 1002.66 | 61.69 | 1830.7 | 159.2 | 1.83 |
| | subcomponent, beta | | | | | | |
| | polypeptide | | | | | | |
| 1434380_at | Diabetic nephropathy-related | BM241271 | 91.08 | 14.75 | 230.44 | 37.81 | 2.53 |
| | gene 1 mRNA, partial | | | | | | |
| | sequence | | | | | | |
| 1434437_x_at | ribonucleotide reductase M2 | AV301324 | 51.35 | 5.57 | 324.97 | 63.16 | 6.33 |
| 1434695_at | RIKEN cDNA 2810047L02 | AV270035 | 61.84 | 11.21 | 208.05 | 25.46 | 3.36 |
| | gene | | | | | | |
| 1434748_at | cytoskeleton associated | BM208103 | 24.36 | 6.06 | 174.75 | 28.18 | 7.17 |
| | protein 2 | | | | | | |
| 1434859_at | uridine monophosphate | BB127793 | 191.28 | 18.19 | 309.99 | 36 | 1.62 |
| | synthetase | | | | | | |
| 1435122_x_at | DNA methyltransferase | BB165431 | 247.53 | 13.07 | 459.38 | 29.9 | 1.86 |
| | (cytosine-5) 1 | | | | | | |
| 1435906_x_at | guanylate nucleotide binding | BE197524 | 66.36 | 7.91 | 225.62 | 53.3 | 3.4 |
| | protein 2 | | | | | | |
| 1436058_at | RIKEN cDNA 2510004L01 | BB132493 | 62.08 | 14.2 | 255.08 | 47.62 | 4.11 |
| | gene | | | | | | |
| 1436349_at | 11 days embryo whole body | BI408855 | 641.52 | 57.67 | 999.98 | 24.3 | 1.56 |
| | cDNA, RIKEN full-length | | | | | | |
| | enriched library, | | | | | | |
| | clone: 2700094K13 | | | | | | |
| | product: unknown EST, full | | | | | | |
| | insert sequence | | | | | | |
| 1436454_x_at | flap structure specific | BB393998 | 466.69 | 46.24 | 762 | 58.32 | 1.63 |
| | endonuclease 1 | | | | | | |
| 1436708_x_at | minichromosome | BB447978 | 127.2 | 15 | 404.48 | 52.01 | 3.18 |
| | maintenance deficient 4 | | | | | | |
| | homolog (S. cerevisiae) | | | | | | |
| 1436905_x_at | lysosomal-associated protein | BB218107 | 441.58 | 60.54 | 709.85 | 55.05 | 1.61 |
| | transmembrane 5 | | | | | | |
| 1436996_x_at | lysozyme | AV066625 | 198.31 | 27.42 | 351.42 | 22.39 | 1.77 |
| 1437309_a_at | replication protein A1 | BB491281 | 1121.92 | 16.63 | 1847.38 | 65.73 | 1.65 |
| 1437313_x_at | high mobility group box 2 | C85885 | 83.26 | 9.86 | 263.68 | 38.12 | 3.17 |
| 1437480_at | RIKEN cDNA 1110001A07 | BB071833 | 156.06 | 21.96 | 332.74 | 38.31 | 2.13 |
| | gene | | | | | | |
| 1437511_x_at | Mid-1-related chloride channel 1 | BB100861 | 299.95 | 13.03 | 403.04 | 12.95 | 1.34 |
| 1437726_x_at | complement component 1, q | BB111335 | 549.17 | 57.73 | 1088.74 | 94.07 | 1.98 |
| | subcomponent, beta | | | | | | |
| | polypeptide | | | | | | |
| 1437874_s_at | hexosaminidase B | AV225808 | 1604.46 | 76.46 | 2311.73 | 185.1 | 1.44 |
| 1438009_at | histone 1, H2ae | W91024 | 792.21 | 49.53 | 2350.02 | 325.15 | 2.97 |
| 1438096_a_at | deoxythymidylate kinase | AV306250 | 299.87 | 17.02 | 497.6 | 46.59 | 1.66 |
| 1438118_x_at | vimentin | AV147875 | 1566.82 | 39.84 | 2049.99 | 78.82 | 1.31 |
| 1438168_x_at | DEAD (Asp-Glu-Ala-Asp) box | AV214253 | 172.85 | 10.5 | 284.04 | 15.81 | 1.64 |
| | polypeptide 39 | | | | | | |
| 1438320_s_at | minichromosome | BB464359 | 261.82 | 11.22 | 1149.59 | 109.86 | 4.39 |
| | maintenance deficient 7 (S. cerevisiae) | | | | | | |
| 1438629_x_at | granulin | AV166504 | 881.67 | 46.89 | 1532.23 | 130.21 | 1.74 |
| 1438852_x_at | minichromosome | BB099487 | 54.91 | 9.64 | 370.87 | 55.94 | 6.75 |
| | maintenance deficient 6 | | | | | | |
| | (MIS5 homolog, S. pombe) | | | | | | |
| | (S. cerevisiae) | | | | | | |
| 1439012_a_at | deoxycytidine kinase | BB030204 | 352.3 | 41.52 | 621.76 | 47.51 | 1.76 |
| 1439269_x_at | minichromosome | BB407228 | 120.49 | 11.02 | 416.51 | 28.23 | 3.46 |
| 1439377_x_at | maintenance deficient 7 (S. cerevisiae) | BB041150 | 53.88 | 10.66 | 237.84 | 22.31 | 4.41 |
| | cell division cycle 20 homolog | | | | | | |
| | (S. cerevisiae) | | | | | | |
| 1439399_a_at | RNA, U22 small nucleolar | BB493265 | 467.35 | 20.35 | 903.88 | 76.79 | 1.93 |
| 1439426_x_at | P lysozyme structural | AV058500 | 172.48 | 32.16 | 339.15 | 13.2 | 1.97 |
| 1439436_x_at | inner centromere protein | BB418702 | 204.9 | 11.82 | 316.61 | 6.35 | 1.55 |
| 1447982_at | RIKEN cDNA 1110008P14 | C79326 | 726.07 | 52.75 | 506.12 | 36.13 | −1.43 |
| | gene | | | | | | |
| 1448118_a_at | cathepsin D | NM_009983 | 2063.01 | 59.36 | 2989.01 | 169.53 | 1.45 |
| 1448127_at | ribonucleotide reductase M1 | BB758819 | 123.55 | 13.68 | 305.63 | 12.37 | 2.47 |
| 1448148_at | granulin | M86736 | 489.13 | 22.2 | 893.61 | 106.39 | 1.83 |
| 1448205_at | cyclin B1 | NM_007629 | 25.79 | 8.24 | 208.5 | 22.25 | 8.08 |
| 1448226_at | ribonucleotide reductase M2 | NM_009104 | 26.71 | 6.51 | 171.03 | 24.04 | 6.4 |
| 1448285_at | regulator of G-protein | NM_009062 | 708.62 | 41.38 | 480.86 | 20.53 | −1.47 |
| | signaling 4 | | | | | | |
| 1448314_at | cell division cycle 2 homolog | NM_007659 | 19.23 | 12.11 | 489.66 | 42.11 | 25.47 |
| | A (S. pombe) | | | | | | |
| 1448380_at | lectin, galactoside-binding, | NM_011150 | 185.76 | 28.51 | 704.21 | 152.74 | 3.79 |
| | soluble, 3 binding protein | | | | | | |
| 1448475_at | olfactomedin-like 3 | NM_133859 | 296.91 | 26.94 | 576.61 | 113.13 | 1.94 |
| 1448591_at | cathepsin S | NM_021281 | 1744.8 | 90.28 | 2804.18 | 195.06 | 1.61 |
| 1448617_at | CD53 antigen | NM_007651 | 229.82 | 14.62 | 336.73 | 23.77 | 1.47 |
| 1448627_s_at | PDZ binding kinase | NM_023209 | 25.18 | 7.4 | 536.43 | 61.81 | 21.31 |
| 1448635_at | SMC2 structural maintenance | NM_008017 | 137.77 | 16.57 | 430.77 | 48.91 | 3.13 |
| | of chromosomes 2-like 1 | | | | | | |
| | (yeast) | | | | | | |
| 1448650_a_at | polymerase (DNA directed), | NM_011132 | 12.22 | 9.49 | 122.62 | 15.13 | 10.03 |
| | epsilon | | | | | | |
| 1448659_at | caspase 7 | NM_007611 | 84.73 | 12.02 | 224.2 | 18.78 | 2.65 |
| 1448694_at | Jun oncogene | NM_010591 | 733.02 | 23.2 | 1030.16 | 20.95 | 1.41 |
| 1448706_at | Traf and Tnf receptor | NM_019551 | 321.63 | 30.35 | 533.7 | 11.47 | 1.66 |
| | associated protein | | | | | | |
| 1448748_at | pleckstrin | AF181829 | 134.25 | 11.27 | 243.04 | 26.33 | 1.81 |
| 1448777_at | minichromosome | NM_008564 | 38.39 | 7.36 | 243.29 | 12.03 | 6.34 |
| | maintenance deficient 2 | | | | | | |
| | mitotin (S. cerevisiae) | | | | | | |
| 1448828_at | SMC6 structural maintenance | AV281575 | 404.1 | 20.91 | 557.29 | 34.69 | 1.38 |
| | of chromosomes 6-like 1 | | | | | | |
| | (yeast) | | | | | | |
| 1448891_at | macrophage scavenger | BC016551 | 234.85 | 57.53 | 430.88 | 50.49 | 1.83 |
| | receptor 2 | | | | | | |
| 1448899_s_at | RAD51 associated protein 1 | BC003738 | 178.77 | 24.33 | 301.74 | 25.49 | 1.69 |
| 1449009_at | T-cell specific GTPase | NM_011579 | 84.46 | 12.17 | 226.65 | 32.32 | 2.68 |
| 1449025_at | interferon-induced protein with | NM_010501 | 268.57 | 28.63 | 1122.15 | 296.98 | 4.18 |
| | tetratricopeptide repeats 3 | | | | | | |
| 1449061_a_at | DNA primase, p49 subunit | J04620 | 74.85 | 9.68 | 245.54 | 13.2 | 3.28 |
| 1449164_at | CD68 antigen | BC021637 | 166.02 | 22.3 | 375.49 | 24.02 | 2.26 |
| 1449172_a_at | lin 7 homolog b (C. elegans) | NM_011698 | 616.36 | 45.32 | 410.69 | 33.88 | −1.5 |
| 1449176_a_at | deoxycytidine kinase | NM_007832 | 430.48 | 15.6 | 670.56 | 44.1 | 1.56 |
| 1449200_at | nucleoporin 155 | BG073833 | 247.44 | 29.8 | 447.23 | 41.8 | 1.81 |
| 1449217_at | caspase 8 associated protein 2 | NM_011997 | 295.61 | 26.55 | 454.46 | 41.74 | 1.54 |
| 1449289_a_at | beta-2 microglobulin | BF715219 | 1924.79 | 71.86 | 3159.06 | 261.82 | 1.64 |
| 1449401_at | complement component 1, q | NM_007574 | 1043.39 | 52.68 | 1900.47 | 248.54 | 1.82 |
| | subcomponent, gamma | | | | | | |
| | polypeptide | | | | | | |
| 1449556_at | histocompatibility 2, T region | NM_010398 | 340.06 | 37.17 | 648.14 | 68.17 | 1.91 |
| | locus 23 | | | | | | |
| 1449687_at | DNA segment, Chr 10, | AU014694 | 1172.52 | 58.84 | 858.48 | 23.33 | −1.37 |
| | ERATO Doi 610, expressed | | | | | | |
| | minichromosome | | | | | | |
| 1449705_x_at | maintenance deficient 3 (S. cerevisiae) | C80350 | 14.94 | 11.11 | 262.43 | 21.98 | 17.56 |
| 1449708_s_at | checkpoint kinase 1 homolog | C85740 | 26.65 | 6.32 | 128.75 | 21.66 | 4.83 |
| | (S. pombe) | | | | | | |
| 1449770_x_at | DNA segment, Chr 16, | N28171 | 979.55 | 39.86 | 722.29 | 42.6 | −1.36 |
| | Brigham & Women's Genetics | | | | | | |
| | 1494 expressed | | | | | | |
| 1449839_at | caspase 3, apoptosis related | BG070529 | 262.22 | 30.95 | 512.26 | 28.08 | 1.95 |
| | cysteine protease | | | | | | |
| 1449977_at | early growth response 4 | NM_020596 | 310.31 | 29.91 | 184.87 | 22.24 | −1.68 |
| 1450033_a_at | signal transducer and | AW214029 | 82.23 | 9.37 | 308.17 | 70.85 | 3.75 |
| | activator of transcription 1 | | | | | | |
| 1450034_at | signal transducer and | AW214029 | 146.98 | 13 | 436.21 | 86.49 | 2.97 |
| | activator of transcription 1 | | | | | | |
| 1450416_at | chromobox homolog 5 | NM_007626 | 496.27 | 13.45 | 816.23 | 62.22 | 1.64 |
| | (Drosophila HP1a) | | | | | | |
| 1450641_at | vimentin | M24849 | 679.69 | 28.8 | 908.66 | 38.48 | 1.34 |
| 1450662_at | testis specific protein kinase 1 | NM_011571 | 580.7 | 14.11 | 445.4 | 11.64 | −1.3 |
| 1450678_at | integrin beta 2 | NM_008404 | 119.27 | 11.84 | 251.74 | 14.65 | 2.11 |
| 1450692_at | kinesin family member 4 | NM_008446 | 26.98 | 10.35 | 385.42 | 93.74 | 14.29 |
| 1450783_at | interferon-induced protein with | NM_008331 | 42.34 | 9.47 | 363 | 107.65 | 8.57 |
| | tetratricopeptide repeats 1 | | | | | | |
| 1450792_at | TYRO protein tyrosine kinase | NM_011662 | 582.01 | 69.33 | 1170.76 | 116.12 | 2.01 |
| | binding protein | | | | | | |
| 1451065_a_at | DEAD (Asp-Glu-Ala-Asp) box | BC020134 | 152.45 | 9.88 | 263.47 | 23.9 | 1.73 |
| | polypeptide 39 | | | | | | |
| 1451080_at | ubiquitin specific protease 1 | BC018179 | 430.16 | 23.89 | 826.91 | 43.76 | 1.92 |
| 1451163_at | Terf1 (TRF1)-interacting | AF214013 | 169.67 | 18.47 | 313.02 | 6.86 | 1.84 |
| | nuclear factor 2 | | | | | | |
| 1451358_a_at | Rac GTPase-activating | AF212320 | 89.65 | 10.97 | 234.99 | 21.94 | 2.62 |
| | protein 1 | | | | | | |
| 1451377_a_at | achalasia, adrenocortical | BC025501 | 130.96 | 12.2 | 258.88 | 11.34 | 1.98 |
| | insufficiency, alacrimia | | | | | | |
| 1451517_at | Rho-related BTB domain | AF420001 | 430.81 | 17.1 | 318.45 | 11.8 | −1.35 |
| | containing 2 | | | | | | |
| 1451599_at | sestrin 2 | AV308638 | 137.61 | 17.29 | 275.4 | 17.08 | 2 |
| 1451683_x_at | histocompatibility 2, K1, K | M34962 | 188.1 | 18.09 | 367.14 | 56.15 | 1.95 |
| | region | | | | | | |
| 1451784_x_at | histocompatibility 2, K1, K | L36068 | 674.7 | 42.78 | 1186.93 | 177.33 | 1.76 |
| | region | | | | | | |
| 1451860_a_at | tripartite motif protein 30 | AF220015 | 45.27 | 4 | 177.33 | 43.2 | 3.92 |
| 1451931_x_at | histocompatibility 2, K1, K | M69068 | 563.81 | 24.9 | 994.32 | 129.69 | 1.76 |
| | region | | | | | | |
| 1452036_a_at | thymopoietin | AA153892 | 318.9 | 16 | 562.6 | 23.6 | 1.76 |
| 1452197_at | SMC4 structural maintenance | AV172948 | 108.34 | 16.5 | 369.02 | 34.23 | 3.41 |
| | of chromosomes 4-like 1 | | | | | | |
| | (yeast) | | | | | | |
| 1452199_at | RIKEN cDNA 2700094F01 | BB667255 | 250.12 | 19.68 | 359.81 | 20.25 | 1.44 |
| | gene | | | | | | |
| 1452241_at | RIKEN cDNA 2810429C13 | BC007170 | 180.73 | 22.23 | 375.99 | 48.84 | 2.08 |
| | gene | | | | | | |
| 1452305_s_at | RIKEN cDNA 2610510J17 | BM230253 | 26.89 | 7.63 | 141.25 | 21.97 | 5.25 |
| | gene | | | | | | |
| 1452313_at | RIKEN cDNA 5930416I19 | AK011167 | 226.72 | 18.66 | 341.31 | 9.07 | 1.51 |
| | gene | | | | | | |
| 1452428_a_at | beta-2 microglobulin | AI099111 | 2166.21 | 54.34 | 3341.96 | 272.64 | 1.54 |
| 1452534_a_at | high mobility group box 2 | X67668 | 88.79 | 16.01 | 313.49 | 39.19 | 3.53 |
| 1452598_at | RIKEN cDNA 2810418N01 | AK013116 | 38.02 | 10.09 | 160.69 | 16.05 | 4.23 |
| | gene | | | | | | |
| 1452659_at | DEK oncogene (DNA binding) | AK007546 | 1095.75 | 85.53 | 1997.63 | 143.77 | 1.82 |
| 1452681_at | deoxythymidylate kinase | AK009220 | 275.51 | 10.2 | 462.59 | 25.26 | 1.68 |
| 1452743_at | polymerase (DNA directed), | AK007693 | 363.6 | 25.15 | 548.48 | 14.54 | 1.51 |
| | epsilon 3 (p17 subunit) | | | | | | |
| 1452954_at | ubiquitin-conjugating enzyme | AV162459 | 17.4 | 4.81 | 142.71 | 20.34 | 8.2 |
| | E2C | | | | | | |
| 1453196_a_at | 2′-5′ oligoadenylate | BQ033138 | 63.81 | 9.98 | 423.26 | 130.06 | 6.63 |
| | synthetase-like 2 | | | | | | |
| 1453314_x_at | RIKEN cDNA 2610039C10 | AK012533 | 153.32 | 10 | 256.77 | 11.1 | 1.67 |
| | gene | | | | | | |
| 1454011_a_at | replication protein A2 | AK011530 | 131.7 | 13.23 | 258.92 | 26.47 | 1.97 |
| 1454268_a_at | cytochrome b-245, alpha | AK018713 | 133.06 | 27.59 | 304.22 | 29.35 | 2.29 |
| | polypeptide | | | | | | |
| 1454694_a_at | topoisomerase (DNA) II alpha | BM211413 | 25.35 | 8.3 | 269.04 | 35.45 | 10.62 |
| 1455715_at | 0 day neonate cerebellum | BB125596 | 257.74 | 36.36 | 155.33 | 13.11 | −1.66 |
| | cDNA, RIKEN full-length | | | | | | |
| | enriched library, | | | | | | |
| | clone: C230080E09 | | | | | | |
| | product: hypothetical protein, | | | | | | |
| | full insert sequence | | | | | | |
| 1455814_x_at | DEAD (Asp-Glu-Ala-Asp) box | AV111502 | 165.56 | 11.84 | 279.16 | 15.24 | 1.69 |
| | polypeptide 39 | | | | | | |
| 1455832_a_at | uridine monophosphate | BE951337 | 177.53 | 11.55 | 326.01 | 16.67 | 1.84 |
| | synthetase | | | | | | |
| 1456055_x_at | polymerase (DNA directed), | BB385244 | 66.86 | 12.15 | 183.22 | 8.73 | 2.74 |
| | delta 1, catalytic subunit | | | | | | |
| 1456292_a_at | vimentin | AV147875 | 444.86 | 17.91 | 609.08 | 37.93 | 1.37 |
| 1456307_s_at | adenylate cyclase 7 | BB746807 | 105.12 | 5.31 | 280.29 | 13.18 | 2.67 |
| 1456567_x_at | granulin | BB000455 | 879.63 | 61.85 | 1444.37 | 113.46 | 1.64 |
| 1459890_s_at | RIKEN cDNA 1110008P14 | C79326 | 2157.39 | 70.4 | 1515.76 | 66.87 | −1.42 |
| | gene | | | | | | |
| 1460168_at | stem-loop binding protein | NM_009193 | 475.9 | 42.77 | 974.34 | 51.05 | 2.05 |
| 1460180_at | hexosaminidase B | NM_010422 | 2038.49 | 61.32 | 2876.86 | 157.71 | 1.41 |
| 1460206_at | GRP1 (general receptor for | NM_019518 | 321.08 | 31.52 | 219.67 | 10.63 | −1.46 |
| | phosphoinositides 1)- | | | | | | |
| | associated scaffold protein | | | | | | |
| 1460218_at | CD52 antigen | NM_013706 | 83.23 | 10.92 | 331.82 | 57.08 | 3.99 |
| 1460716_a_at | core binding factor beta | NM_022309 | 684.76 | 40.76 | 1081.73 | 110.84 | 1.58 |

DCHIP parameters are described in Materials and Methods.
Fold change indicates fold change in CK-p25 mice over uninduced controls.
Baseline refers to the uninduced control group, while exp refers to the p25 induced group.
SE refers to standard error.
Note that specific fold change values differ from Table 1 values, which were obtained using GCOS software (Affymetrix).

TABLE 3
Complete list of cell cycle and DNA damage related genes with altered
expression in 2 week induced CK-p25 mice compared to uninduced controls.

| | | | | baseline | | exp | |
|---|---|---|---|---|---|---|---|
| probe | | | baseline | mean | exp | mean | FOLD |
| set | gene | Accession | mean | SE | mean | SE | CHANGE |
| 1456055_x_at | polymerase (DNA directed), delta | BB385244 | 66.86 | 12.15 | 183.22 | 8.73 | 2.74 |
| | 1, catalytic subunit | | | | | | |
| 1454694_a_at | topoisomerase (DNA) II alpha | BM211413 | 25.35 | 8.3 | 269.04 | 35.45 | 10.62 |
| 1454011_a_at | replication protein A2 | AK011530 | 131.7 | 13.23 | 258.92 | 26.47 | 1.97 |
| 1452954_at | ubiquitin-conjugating enzyme | AV162459 | 17.4 | 4.81 | 142.71 | 20.34 | 8.2 |
| | E2C | | | | | | |
| 1452534_a_at | high mobility group box 2 | X67668 | 88.79 | 16.01 | 313.49 | 39.19 | 3.53 |
| 1452197_at | SMC4 structural maintenance of | AV172948 | 108.34 | 16.5 | 369.02 | 34.23 | 3.41 |
| | chromosomes 4-like 1 (yeast) | | | | | | |
| 1451599_at | sestrin 2 | AV308638 | 137.61 | 17.29 | 275.4 | 17.08 | 2 |
| 1451163_at | Terf1 (TRF1)-interacting nuclear | AF214013 | 169.67 | 18.47 | 313.02 | 6.86 | 1.84 |
| | factor 2 | | | | | | |
| 1450416_at | chromobox homolog 5 | NM_007626 | 496.27 | 13.45 | 816.23 | 62.22 | 1.64 |
| | (Drosophila HP1a) | | | | | | |
| 1449839_at | caspase 3, apoptosis related | BG070529 | 262.22 | 30.95 | 512.26 | 28.08 | 1.95 |
| | cysteine protease | | | | | | |
| 1449708_s_at | checkpoint kinase 1 homolog (S. pombe) | C85740 | 26.65 | 6.32 | 128.75 | 21.66 | 4.83 |
| 1449705_x_at | minichromosome maintenance | C80350 | 14.94 | 11.11 | 262.43 | 21.98 | 17.56 |
| | deficient 3 (S. cerevisiae) | | | | | | |
| 1449061_a_at | DNA primase, p49 subunit | J04620 | 74.85 | 9.68 | 245.54 | 13.2 | 3.28 |
| 1448899_s_at | RAD51 associated protein 1 | BC003738 | 178.77 | 24.33 | 301.74 | 25.49 | 1.69 |
| 1448777_at | minichromosome maintenance | NM_008564 | 38.39 | 7.36 | 243.29 | 12.03 | 6.34 |
| | deficient 2 mitotin (S. cerevisiae) | | | | | | |
| 1448694_at | Jun oncogene | NM_010591 | 733.02 | 23.2 | 1030.16 | 20.95 | 1.41 |
| 1448650_a_at | polymerase (DNA directed), | NM_011132 | 12.22 | 9.49 | 122.62 | 15.13 | 10.03 |
| | epsilon | | | | | | |
| 1448635_at | SMC2 structural maintenance of | NM_008017 | 137.77 | 16.57 | 430.77 | 48.91 | 3.13 |
| | chromosomes 2-like 1 (yeast) | | | | | | |
| 1448314_at | cell division cycle 2 homolog A | NM_007659 | 19.23 | 12.11 | 489.66 | 42.11 | 25.47 |
| | (S. pombe) | | | | | | |
| 1448226_at | ribonucleotide reductase M2 | NM_009104 | 26.71 | 6.51 | 171.03 | 24.04 | 6.4 |
| 1448205_at | cyclin B1 | NM_007629 | 25.79 | 8.24 | 208.5 | 22.25 | 8.08 |
| 1448127_at | ribonucleotide reductase M1 | BB758819 | 123.55 | 13.68 | 305.63 | 12.37 | 2.47 |
| 1439436_x_at | inner centromere protein | BB418702 | 204.9 | 11.82 | 316.61 | 6.35 | 1.55 |
| 1439377_x_at | cell division cycle 20 homolog (S. cerevisiae) | BB041150 | 53.88 | 10.66 | 237.84 | 22.31 | 4.41 |
| 1439269_x_at | minichromosome maintenance | BB407228 | 120.49 | 11.02 | 416.51 | 28.23 | 3.46 |
| | deficient 7 (S. cerevisiae) | | | | | | |
| 1438852_x_at | minichromosome maintenance | BB099487 | 54.91 | 9.64 | 370.87 | 55.94 | 6.75 |
| | deficient 6 (MIS5 homolog, S. pombe) | | | | | | |
| | (S. cerevisiae) | | | | | | |
| 1438320_s_at | minichromosome maintenance | BB464359 | 261.82 | 11.22 | 1149.59 | 109.86 | 4.39 |
| | deficient 7 (S. cerevisiae) | | | | | | |
| 1437313_x_at | high mobility group box 2 | C85885 | 83.26 | 9.86 | 263.68 | 38.12 | 3.17 |
| 1437309_a_at | replication protein A1 | BB491281 | 1121.92 | 16.63 | 7.38 | 65.73 | 1.65 |
| 1436708_x_at | minichromosome maintenance | BB447978 | 127.2 | 15 | 404.48 | 52.01 | 3.18 |
| | deficient 4 homolog (S. cerevisiae) | | | | | | |
| 1436454_x_at | flap structure specific | BB393998 | 466.69 | 46.24 | 762 | 58.32 | 1.63 |
| | endonuclease 1 | | | | | | |
| 1435122_x_at | DNA methyltransferase | BB165431 | 247.53 | 13.07 | 459.38 | 29.9 | 1.86 |
| | (cytosine-5) 1 | | | | | | |
| 1434437_x_at | ribonucleotide reductase M2 | AV301324 | 51.35 | 5.57 | 324.97 | 63.16 | 6.33 |
| 1434079_s_at | minichromosome maintenance | BB699415 | 87.64 | 12.77 | 432.69 | 16.22 | 4.94 |
| | deficient 2 mitotin (S. cerevisiae) | | | | | | |
| 1430811_a_at | cell division cycle associated 1 | AK010351 | 91.63 | 13.29 | 207.53 | 18.55 | 2.26 |
| 1427724_at | topoisomerase (DNA) II alpha | U01919 | 47.19 | 16.53 | 147.38 | 22.73 | 3.12 |
| 1427275_at | SMC4 structural maintenance of | BI665568 | 159.11 | 13.89 | 607.23 | 74.11 | 3.82 |
| | chromosomes 4-like 1 (yeast) | | | | | | |
| 1426838_at | polymerase (DNA-directed), delta | AK010805 | 211.19 | 23.1 | 402.22 | 21.31 | 1.9 |
| | 3, accessory subunit | | | | | | |
| 1426817_at | antigen identified by monoclonal | X82786 | 28.14 | 9.18 | 245.79 | 41.74 | 8.74 |
| | antibody Ki 67 | | | | | | |
| 1426653_at | minichromosome maintenance | BI658327 | 63.81 | 18.71 | 198.02 | 8.17 | 3.1 |
| | deficient 3 (S. cerevisiae) | | | | | | |
| 1425753_a_at | uracil-DNA glycosylase | BC004037 | 32.27 | 6.23 | 146.89 | 9.68 | 4.55 |
| 1424638_at | cyclin-dependent kinase inhibitor | AK007630 | 254.59 | 49.08 | 574.02 | 126.42 | 2.25 |
| | 1A (P21) | | | | | | |
| 1424629_at | breast cancer 1 | U31625 | 31.3 | 8.35 | 150.19 | 17.3 | 4.8 |
| 1424321_at | replication factor C (activator 1) 4 | BC003335 | 113.26 | 14.16 | 305.38 | 18.87 | 2.7 |
| 1424144_at | retroviral integration site 2 | AF477481 | 22.25 | 14.75 | 489.72 | 40.35 | 22.01 |
| 1423847_at | RIKEN cDNA 2810406C15 gene | BC025460 | 192.64 | 7.96 | 339.89 | 15.8 | 1.76 |
| 1423714_at | ASF1 anti-silencing function 1 | BC003428 | 31.92 | 11.86 | 160.19 | 15.08 | 5.02 |
| | homolog B (S. cerevisiae) | | | | | | |
| 1423293_at | replication protein A1 | BM244983 | 517.64 | 29.86 | 845.63 | 39.32 | 1.63 |
| 1423241_a_at | transcription factor Dp 1 | BG075396 | 480.48 | 20.48 | 747.43 | 58.29 | 1.56 |
| 1423100_at | FBJ osteosarcoma oncogene | AV026617 | 1512.22 | 109.28 | 1.52 | 46.33 | −1.45 |
| 1422946_a_at | DNA methyltransferase | NM_010066 | 413.52 | 18.37 | 741.17 | 39.72 | 1.79 |
| | (cytosine-5) 1 | | | | | | |
| 1422535_at | cyclin E2 | AF091432 | 108.4 | 21.69 | 507.25 | 53.46 | 4.68 |
| 1422460_at | MAD2 (mitotic arrest deficient, | NM_019499 | 147.87 | 16.85 | 323.31 | 12.94 | 2.19 |
| | homolog)-like 1 (yeast) | | | | | | |
| 1422016_a_at | centromere autoantigen H | BC025084 | 11.16 | 4.61 | 160.93 | 18.52 | 14.42 |
| 1421731_a_at | flap structure specific | NM_007999 | 137.71 | 20.22 | 351.18 | 27.44 | 2.55 |
| | endonuclease 1 | | | | | | |
| 1420028_s_at | minichromosome maintenance | C80350 | 35.85 | 7.21 | 386.12 | 33 | 10.77 |
| | deficient 3 (S. cerevisiae) | | | | | | |
| 1419943_s_at | cyclin B1 | AU015121 | 21.93 | 7.08 | 134.26 | 22.88 | 6.12 |
| 1419838_s_at | polo-like kinase 4 (Drosophila) | AI385771 | 62.68 | 9.98 | 186.45 | 11.33 | 2.97 |
| 1419270_a_at | deoxyuridine triphosphatase | AF091101 | 479.7 | 42.4 | 794.29 | 51.19 | 1.66 |
| 1418369_at | DNA primase, p49 subunit | J04620 | 151.7 | 12.93 | 416.33 | 24.11 | 2.74 |
| 1418281_at | RAD51 homolog (S. cerevisiae) | NM_011234 | 1.82 | 9.76 | 386.38 | 54.82 | 212.33 |
| 1418203_at | phorbol-12-myristate-13-acetate- | NM_021451 | 37.55 | 10.11 | 278.08 | 46.85 | 7.4 |
| | induced protein 1 | | | | | | |
| 1418036_at | DNA primase, p58 subunit | NM_008922 | 110.45 | 13.1 | 223.34 | 15.06 | 2.02 |
| 1417947_at | proliferating cell nuclear antigen | BC010343 | 1269.31 | 98.2 | 3142.66 | 162.7 | 2.48 |
| 1417938_at | RAD51 associated protein 1 | BC003738 | 26.57 | 5.13 | 359.98 | 46.41 | 13.55 |
| 1417910_at | cyclin A2 | X75483 | 45.28 | 10.2 | 342.45 | 34.42 | 7.56 |
| 1417878_at | E2F transcription factor 1 | NM_007891 | 65.86 | 15.12 | 360.15 | 28.76 | 5.47 |
| 1417830_at | SMC (structural maintenance of | BB156359 | 650.81 | 36.6 | 881.88 | 37.39 | 1.36 |
| | chromosomes 1)-like 1 (S. cerevisiae) | | | | | | |
| 1417541_at | helicase, lymphoid specific | NM_008234 | 16.62 | 3.97 | 179.78 | 25.82 | 10.82 |
| 1417506_at | geminin | NM_020567 | 121.76 | 17.02 | 309.1 | 16.65 | 2.54 |
| 1417503_at | replication factor C (activator 1) 2 | NM_020022 | 520.32 | 26.11 | 737.8 | 35.95 | 1.42 |
| 1417458_s_at | CDC28 protein kinase regulatory | NM_025415 | 76.69 | 6.74 | 443.96 | 37.63 | 5.79 |
| | subunit 2 | | | | | | |
| 1416915_at | mutS homolog 6 (E. coli) | U42190 | 197.78 | 9.83 | 318.35 | 12.04 | 1.61 |
| 1416773_at | wee 1 homolog (S. pombe) | NM_009516 | 358.95 | 18.28 | 494.28 | 21.48 | 1.38 |
| 1416698_a_at | CDC28 protein kinase 1 | NM_016904 | 145 | 20.37 | 393.4 | 22.1 | 2.71 |
| 1416641_at | ligase I, DNA, ATP-dependent | NM_010715 | 133.63 | 14.08 | 535.21 | 55.86 | 4.01 |
| 1416575_at | cell division cycle 45 homolog (S. cerevisiae)- | NM_009862 | 24.19 | 5.77 | 151.32 | 16.09 | 6.26 |
| | like | | | | | | |
| 1416492_at | cyclin E1 | NM_007633 | 124.08 | 12.81 | 253.43 | 19.98 | 2.04 |
| 1416433_at | replication protein A2 | BC004578 | 154.93 | 15.9 | 345.73 | 29.37 | 2.23 |
| 1416251_at | minichromosome maintenance | NM_008567 | 167.95 | 26.1 | 1346.48 | 120.1 | 8.02 |
| | deficient 6 (MIS5 homolog, S. pombe) | | | | | | |
| | (S. cerevisiae) | | | | | | |
| 1416214_at | minichromosome maintenance | BC013094 | 137.85 | 13.4 | 447.68 | 30.03 | 3.25 |
| | deficient 4 homolog (S. cerevisiae) | | | | | | |
| 1416031_s_at | minichromosome maintenance | NM_008568 | 104.16 | 16.5 | 404.63 | 38.45 | 3.88 |
| | deficient 7 (S. cerevisiae) | | | | | | |
| 1415899_at | Jun-B oncogene | NM_008416 | 1458.85 | 63.86 | 1007.27 | 55.48 | −1.45 |
| 1415878_at | ribonucleotide reductase M1 | BB758819 | 117.53 | 21.29 | 362.03 | 36.02 | 3.08 |

The list of genes was compiled based on the gene ontology (GO) structure files, by combining the altered gene lists from the functional groups listed on the top of the table. DCHIP parameters are described in Materials and Methods. Fold change indicates fold change in CK-p25 mice over uninduced controls. Baseline refers to the uninduced control group, while exp refers to the p25 induced group. SE refers to standard error. Note that specific fold change values differ from Table 1 values, which were obtained using GCOS software (Affymetrix).

### Experiment 2: p25 Induction Results in Aberrant Expression of Cell Cycle Proteins

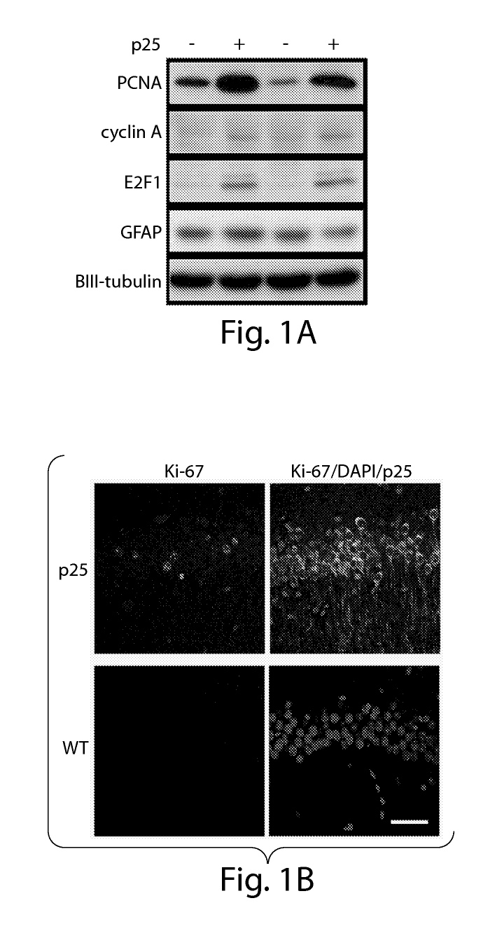

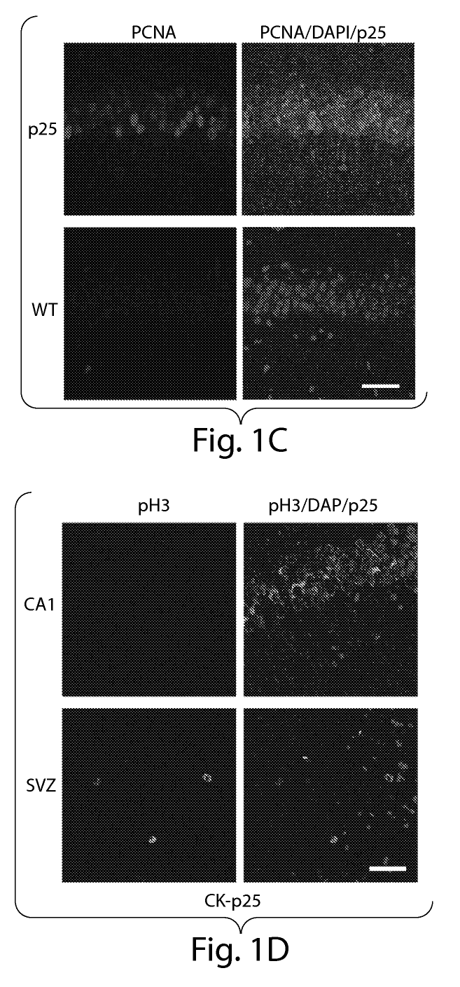

We examined various cell cycle proteins in CK-p25 mouse brains to confirm their aberrant upregulation as suggested by the microarray analyses. Protein levels of PCNA, E2F-1, and Cyclin A were upregulated compared to WT controls (FIG. 1A). There was no change in levels of glial fibrillary acidic protein (GFAP), in line with the absence of neurodegeneration at this period of induction Immunostaining clearly demonstrated robust increases in Ki-67 and PCNA immunoreactivity in p25-expressing adult neurons which were identified by the GFP signal (FIGS. 1B and 1C). Importantly, only neurons expressing p25-GFP were found to have increased levels of cell cycle markers, while no neurons expressed these markers in WT mice. Some nonneuronal cells stained positively for these cell cycle markers (e.g., in the subventricular zone) in both p25 and WT brains (data not shown), reflecting non-pathological cell cycle activity. In addition, we observed that a subset of p25-GFP neurons incorporated bromodeoxyuridine (BrdU), indicating DNA synthesis activity (data not shown). On the other hand, p25-GFP expressing neurons were not immunoreactive for the mitotic marker phospho(pS10)-Histone H3, indicating the absence of mitotic cell cycle activity (FIG. 1D). Our results show that p25 induction results in aberrant expression of cell cycle proteins in neurons, as well as aberrant cell cycle activity.

### Experiment 3: p25 Induction Results in Double Strand DNA Breaks

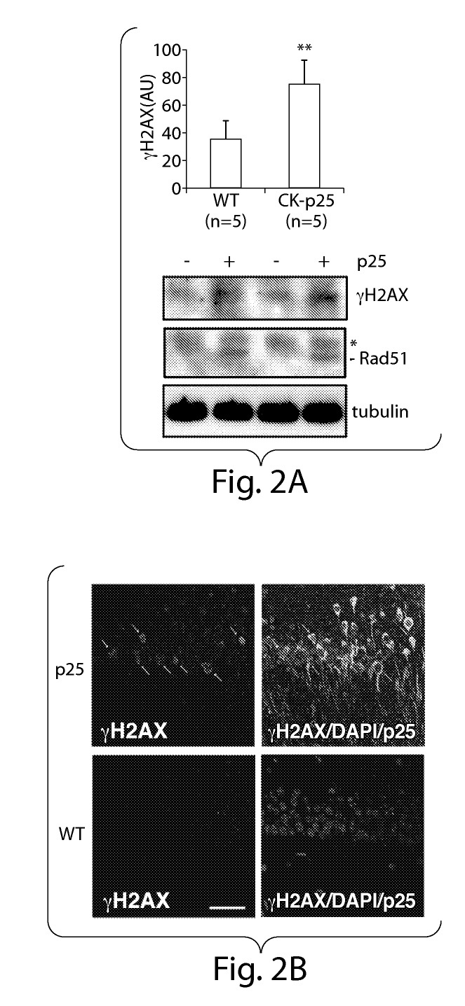

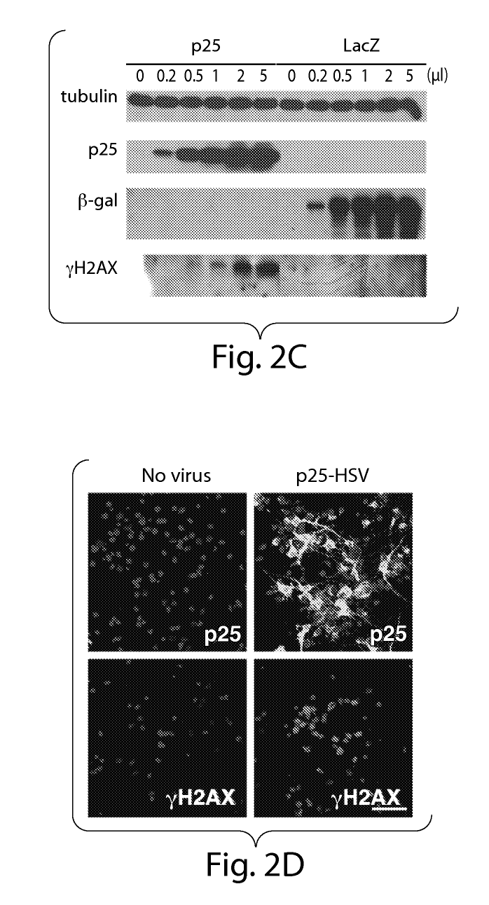

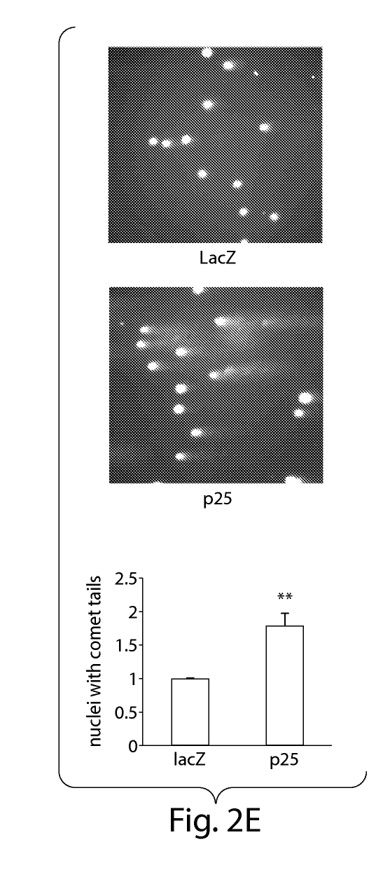

The microarray analyses showed that p25 expression induced many genes involved in the double strand DNA break response. To determine whether double stranded DNA breaks occur in the CK-p25 mice, brains from 2-week induced mice were examined using the double strand break marker phospho-serine 129 histone H2AX (γH2AX). Robust γH2AX immunoreactivity was detected both biochemically (FIG. 2A) and by staining, revealing that γH2AX immunoreactivity was specific to p25-GFP expressing neurons (FIG. 2B). γH2AX staining was undetectable in the WT brain neurons. The double strand DNA break response protein Rad51 was also found to be upregulated in CK-p25 brains (FIG. 2A).

We examined whether p25 mediated induction of double strand breaks could be recapitulated in cultured primary neurons using herpes simplex virus (HSV)-mediated overexpression of p25. Expression of p25 in primary neurons also resulted in robust generation of γH2AX (FIGS. 2C and 2D). To provide physical proof of DNA damage, primary neurons overexpressing p25 were analyzed for DNA strand breaks using single cell gel electrophoresis (comet assay)(Dhawan et al., 2001). We observed that nuclei of p25 overexpressing neurons displayed a ˜1.8-fold higher incidence of comet tails indicative of DNA containing single or double strand breaks (FIG. 2E). These results demonstrate that expression of p25 induces DNA strand breaks in neurons.

### Experiment 4: Double Strand DNA Damage and Cell Cycle Reentry are Tightly Associated and Precede Neuronal Death

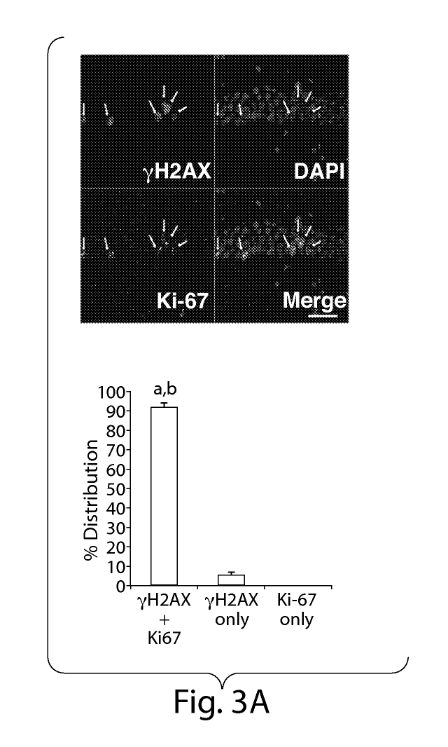

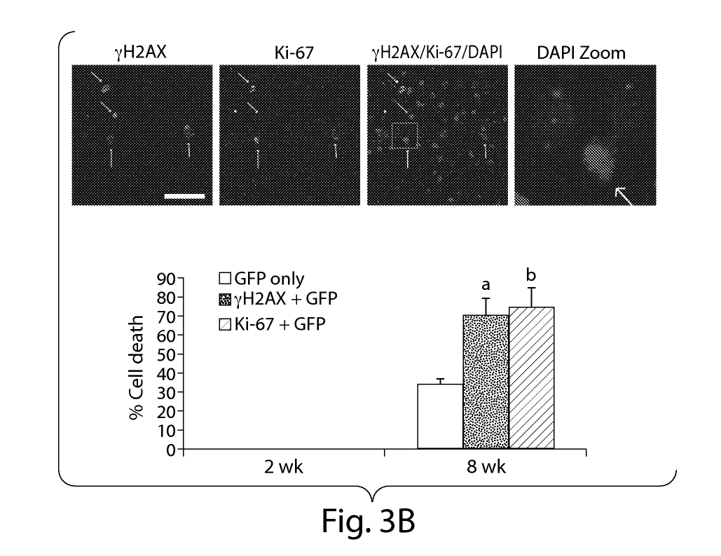

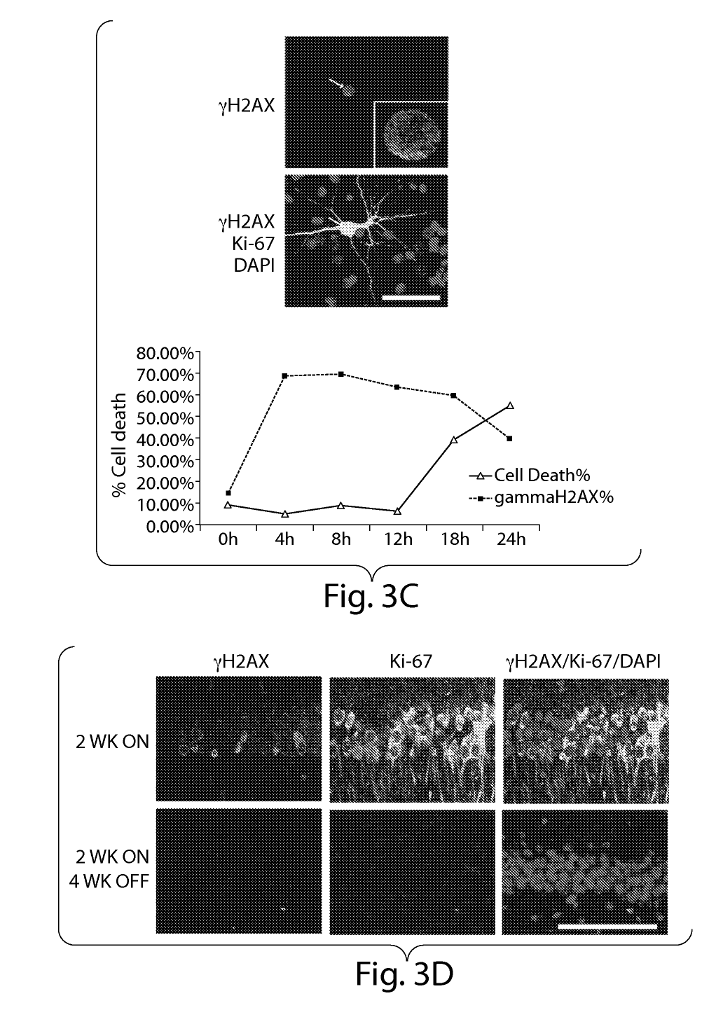

Co-staining with γH2AX and Ki-67 in CK-p25 mice revealed that the same neurons undergoing aberrant expression of cell cycle proteins also exhibited double strand DNA breaks at a high rate of concurrency (92.3±2.7% S.D.), suggesting that the two events are tightly linked (FIG. 3A). In CK-p25 mice induced for 8 weeks (a period when massive neurodegeneration is evident (Cruz et al., 2003)), both the DNA damage marker γH2AX and cell cycle marker Ki-67 were each associated with degenerative nuclei (shrunken or condensed nuclei, or nuclei with invaginations) (FIG. 3B). Over 70% of CA1 neurons in CK-p25 mice that were positive for both p25-GFP and γH2AX, or both p25-GFP and Ki-67 had degenerative nuclei compared to only 34% of neurons positive for p25-GFP alone (FIG. 3B). A time course measurement of incidence of γH2AX immunoreactivity and cell death induced by overexpression of p25-GFP indicated that γH2AX signal was observed as early as 4 hours following p25 transfection, while neuronal death (scored by nuclear and neuritic integrity as described in Methods) was initially observed at 18 hours posttransfection (FIG. 3C). Interestingly, in CK-p25 mice subjected to p25 expression for 2 weeks followed by suppression of p25 expression for 4 weeks (by feeding a doxycycline diet), we observed that γH2AX signal was abrogated (FIG. 3D), while no signs of neuronal loss were observed (Fischer et al., 2005). This indicates that the degree of γH2AX formation observed by 2 weeks is reversible, and that γH2AX formation in CK-p25 mice precedes and is not secondary to cell death.

Collectively, our results demonstrate that cell cycle and DNA damage events are tightly correlated with each other, and that they precede cell death in neurons with p25 accumulation.

Experiment 5: p25 Interacts with and Inhibits HDAC1

Having observed a tight association of cell cycle protein expression and DNA damage in CK-p25 mice, we considered whether a common mechanism may underlie these events. As both gene transcription and susceptibility to DNA damage are known to be tightly linked to the chromatin state, we considered the involvement of HDACs in the induction of aberrant neuronal cell cycle expression and DNA damage by p25/Cdk5 Inhibition of HDACs can potently induce gene transcription, and studies in cancer cell lines have established that inhibition of HDACs can also increase accessibility of DNA to DNA damaging agents (Cerra et al., 2006).

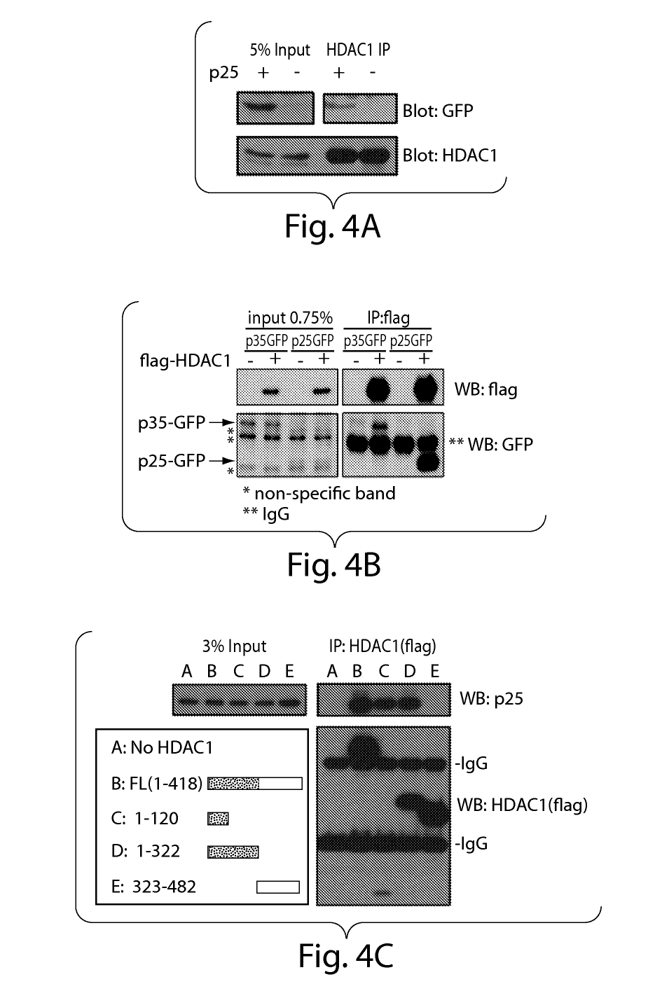

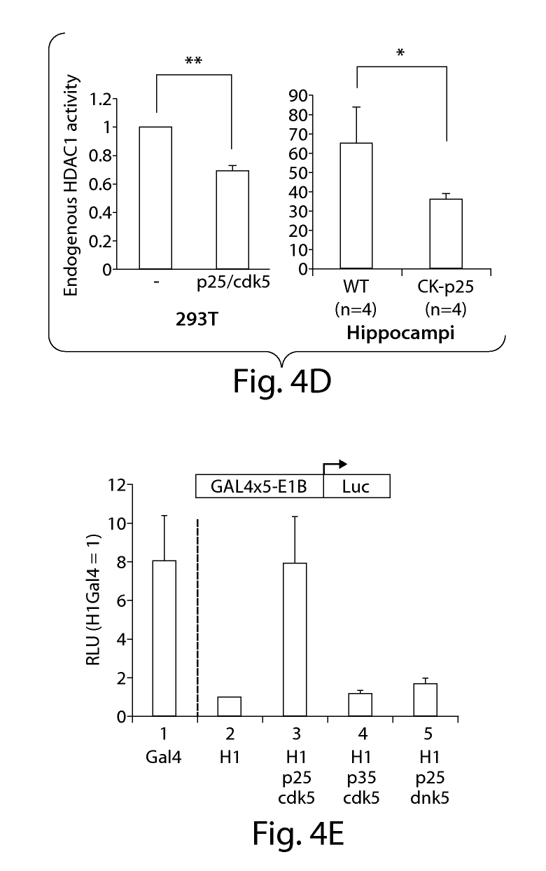

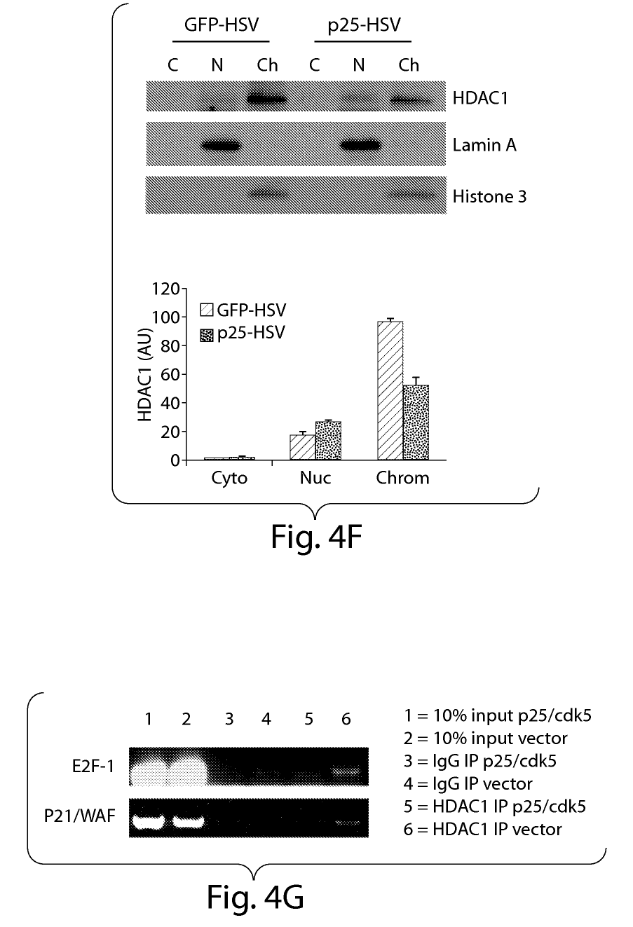

Of particular interest is HDAC1, based on its reported role in transcriptional repression of cell cycle related genes such as p21/WAF, cyclins A, D, and E, and cdc25A (Brehm et al., 1998; Iavarone and Massague, 1999; Lagger et al., 2002; Stadler et al., 2005; Stiegler et al., 1998). We determined that in forebrains of CK-p25 mice induced for 2 weeks, p25 interacted with HDAC1 in vivo (FIG. 4A). Interaction with HDAC1 was observed with both p25 and p35 co-transfected in 293T cells (FIG. 4B). Interestingly, HDAC1 had an over 12-fold higher degree of interaction with p25, compared to the physiological, non-cleaved p35 (FIG. 4B) which does not exert neurotoxicity. The preferential binding of HDAC1 with the pathological molecule p25, compared to p35, raised the interesting possibility that the p25-HDAC1 interaction may have deleterious consequences.

We further characterized the interaction by identifying the interaction domains. To this end, we generated multiple HDAC1 fragments spanning the entire protein, the C terminal region, the N terminal region containing the catalytic domain, or a small N-terminal region within the catalytic domain. By examining the ability of these fragments to coimmunoprecipitate full length p25, we mapped the interaction domain of p25 and HDAC1 to an N-terminal region within the histone deacetylase catalytic domain (FIG. 4C).

The interaction of p25 with the HDAC1 catalytic domain implied that p25/Cdk5 may affect the enzymatic activity and/or the function of HDAC1. We found that overexpression of p25 and Cdk5 in 293T cells resulted in a significant decrease in endogenous HDAC1 activity (FIG. 4D). Importantly, inhibitory effects on endogenous HDAC1 activity were confirmed in vivo in hippocampi from CK-p25 mice compared to WT mice (FIG. 4D). Similar effects on HDAC1 activity were observed in primary neurons infected with p25-HSV (data not shown). To determine whether this was linked to increased HDAC1 repressor activity, we coexpressed p25 and Cdk5 with HDAC1-Ga14 in a luciferase reporter system. Fusion of HDAC1 with Ga14 significantly repressed Ga14 transcriptional activity (Nagy et al., 1997) (lane 2 vs. 1, FIG. 4E); however, co-expression with p25 increased HDAC1-Ga14-induced reporter activity 7.9-fold, indicating decreased repression by HDAC1 (lane 3). Importantly, this effect was not observed with p35/cdk5 or with p25 plus dominant negative cdk5 (lanes 4 and 5), indicating that the inhibitory effect on HDAC1 transcriptional repression was specific to p25 and not p35, and that it required cdk5 activity.

It has been reported that inhibition of HDAC catalytic activity results in the loss of HDAC1 association with the p21/WAF1 promotor region (Gui et al., 2004). Therefore, we investigated whether p25/cdk5 could inhibit the association of HDAC1 from the promotor of p21/WAF1 and other cell cycle related genes. First, we examined whether overexpression of p25 could affect the overall chromatin association of HDAC1 in primary neurons. We observed that HSV-mediated overexpression of p25 led to a 46% decrease in chromatin-bound HDAC1, and a 49.1% increase in the nucleoplasmic, non-chromatin-bound fraction of HDAC1 (FIG. 4F). Next, we carried out HDAC1 chromatin immunoprecipitation experiments in 293T cells transfected with p25/cdk5 or a vector control to examine the association of HDAC1 with the core promotor regions of p21/WAF1 and E2F-1 (FIG. 4G). We found that overexpression of p25/cdk5 resulted in a loss of HDAC1 association with p21/WAF1 and E2F-1 promoters. As HDAC1 activity associated with specific promotor regions is linked with their repression, our result suggested that p25/cdk5 mediated loss of HDAC1 activity and association with promotor regions for cell cycle related genes may account for the aberrant expression of cell cycle related genes observed in the CK-p25 mice.

Collectively, our results demonstrate that p25/cdk5 inhibits multiple facets of HDAC1 function, including histone deacetylase activity, transcriptional repressor activity, and association with chromatin and specific promotor regions.

### Experiment 6: Inhibition of HDAC1 Induces DNA Damage, Cell Cycle Reentry, and Death

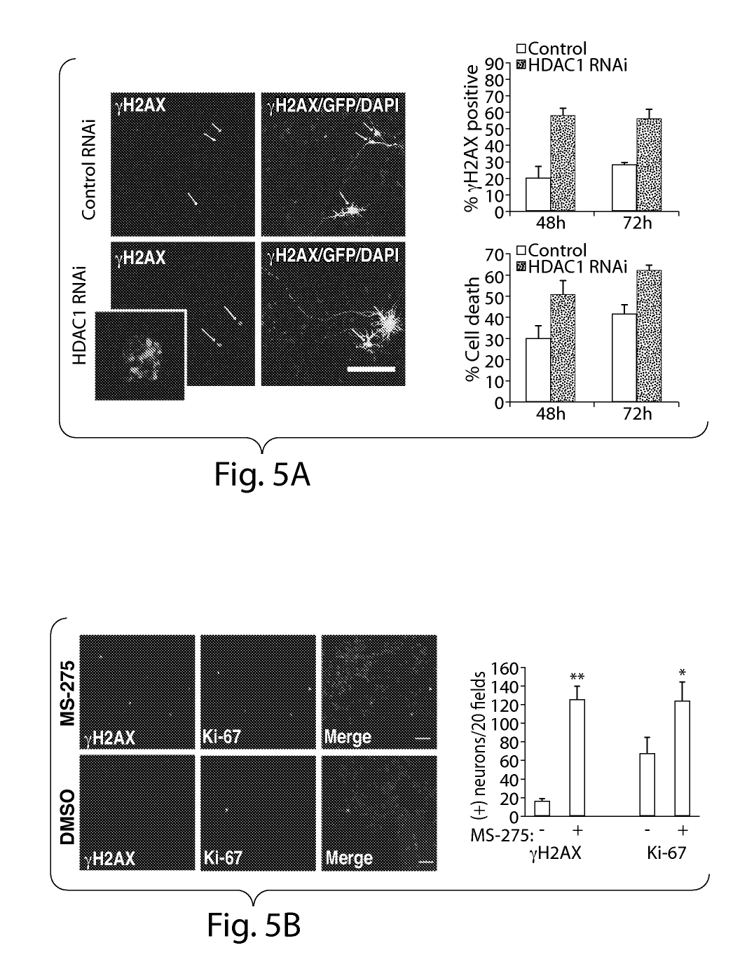

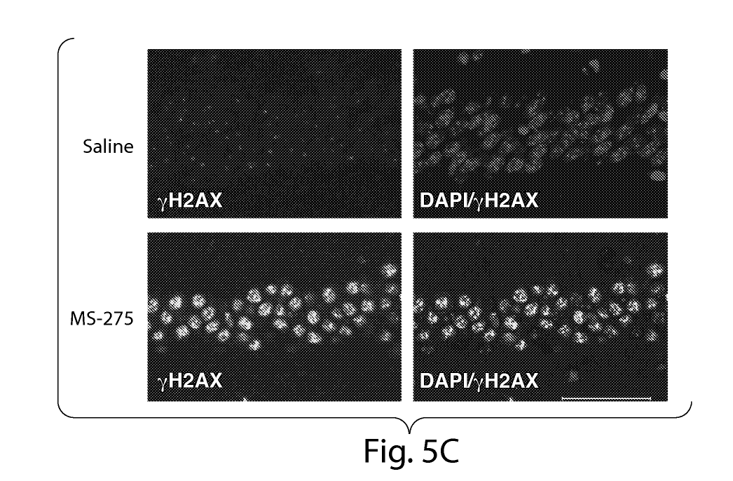

Our findings raised the possibility that p25/cdk5 may cause both cell cycle reentry and DNA damage through inhibition of HDAC1 activity. We examined the effects of siRNA-mediated knockdown or pharmacological inhibition of HDAC1. Knockdown of HDAC1 with a previously utilized sequence (Ishizuka and Lazar, 2003) resulted in a significant increase in double strand DNA breaks and cell death compared to the random sequence control (FIG. 5A). In addition, treatment of primary neurons with 1 μM of the class I HDAC inhibitor MS-275, which results in over 70% inhibition of HDAC1 activity with negligible effects on HDAC3 and HDAC8 (Hu et al., 2003), was sufficient to increase double strand DNA breaks (8.1 fold increase) and stimulate the aberrant expression of Ki-67 (1.8 fold increase) compared to controls (FIG. 5B). These results demonstrate that inhibition of HDAC1 in neurons can induce double strand DNA breaks and cell cycle reentry.

Furthermore, daily intraperitoneal injection of high doses of the HDAC1 inhibitor MS-275 (50 mg/kg) for 5 days in WT mice resulted in a dramatic formation of γH2AX in CA1 neurons, which was not seen with saline injection (FIG. 5C). In contrast to previous studies using the non-selective HDAC inhibitors sodium butyrate and trichostatin A (Fischer et al., 2007; Levenson et al., 2004), MS-275 also impaired associative learning capability in WT mice in a dose dependent manner, as examined using a contextual fear conditioning paradigm (FIG. 8). These results provide support that loss of HDAC1 activity can cause DNA damage, neurodegeneration, and neurologic defects in vivo.

Experiment 7: HDAC1 Gain-of-Function Rescues against DNA Damage and Neuronal Death in Cultured Neurons and In vivo

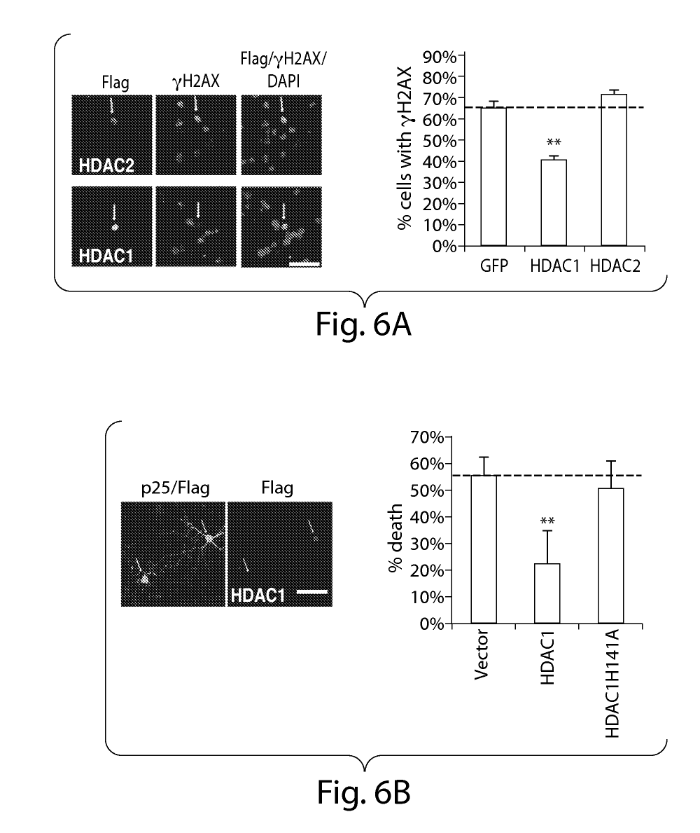

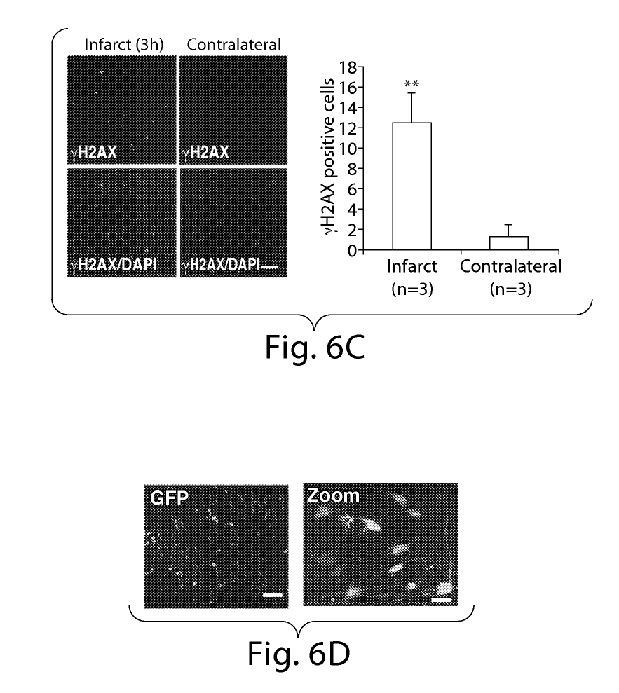

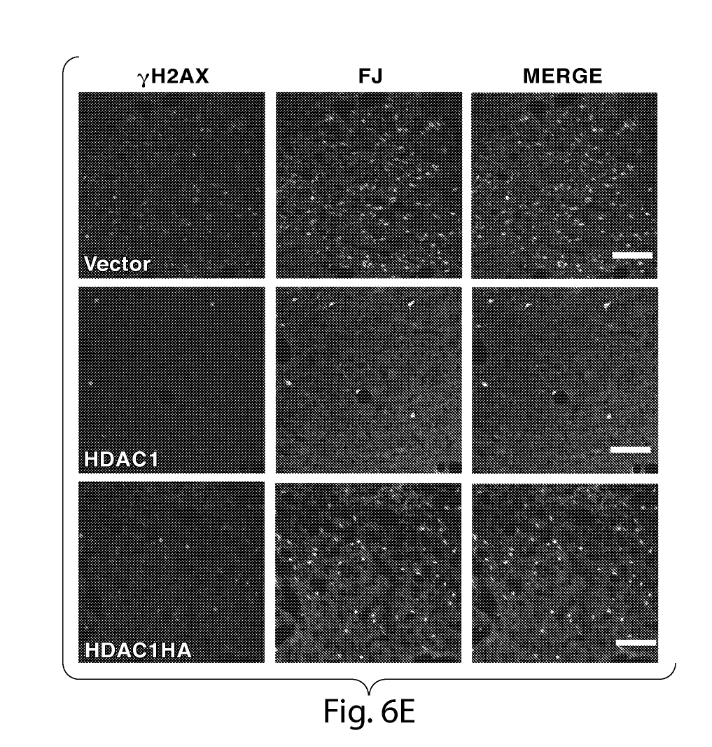

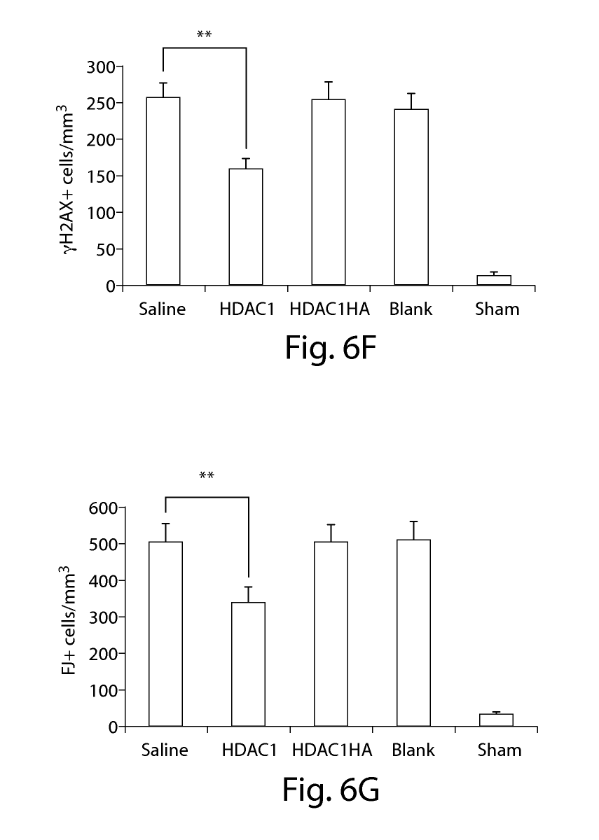

Having demonstrated that inhibition of HDAC1 is sufficient to induce DNA double strand breaks and aberrant cell cycle activity, we examined whether restoration of HDAC1 function by overexpression can attenuate p25-mediated DNA damage and neurotoxicity. To this end, we overexpressed HDAC1 or control constructs followed by viral expression of p25 at a high rate of infection (>80%). Overexpression of HDAC1, but not HDAC2, decreased the percentage of neurons positive for p25-induced γH2AX by 37.9% compared to GFP control (FIG. 6A). We also examined whether co-expression of HDAC1 could rescue against cell death induced by transfection with p25-GFP. Co-expression of HDAC1, but not catalytically dead mutant HDAC1 (HDAC1 H141A), rescued against p25-mediated neuronal death by 59.8% compared to control (FIG. 6B). These results demonstrate that restoring HDAC1 activity can rescue against p25-mediated DNA damage and death.

Next, we sought to examine whether our findings could be recapitulated in an established in vivo model for stroke, i.e., rats subjected to transient forebrain ischemia. We and other groups have previously demonstrated the involvement of p25 in this model (Garcia-Bonilla et al., 2006; Wang et al., 2003; Wen et al., 2007). Also, p25 is upregulated in human postmortem brains following ischemic stroke (Mitsios et al., 2007). Furthermore, induction of cell cycle markers such as Cyclin A, PCNA, and E2F-1, which were upregulated in our p25 mice (FIG. 1), have previously been reported in rodent models for stroke/ischemia (Rashidian et al., 2007).

Therefore, we examined whether γH2AX levels are upregulated as well in this model. Brains from rats subjected to unilateral transient forebrain ischemia for various periods were examined for γH2AX immunoreactivity. Increased γH2AX immunoreactivity was observed as early as three hours post-ischemia in the infarct region (FIG. 6C). Significant levels of γH2AX were not observed in ipsilateral non-infarct region (not shown) or the contralateral hemisphere (FIG. 6C).

We examined whether overexpression of HDAC1 conferred neuroprotection in this model. To this end, Sprague Dawley rats were injected with saline, blank HSV, HSV-HDAC1, or HSV-HDAC1H141A catalytic-dead mutant, into the striatum, which resulted in robust neuronal expression of constructs (FIG. 6D). After 24 hours, rats were subjected to bilateral transient forebrain ischemia. Six days later, brain sections were stained with γH2AX and Fluoro-Jade to label degenerating neurons. We observed that HSV-mediated overexpression of HDAC1 in the striatum resulted in a 38% reduction in γH2AX-positive neurons in the striatum compared to blank HSV, while the HDAC1H141A mutant did not confer neuroprotection (FIGS. 6E and 6F). In addition, the number of degenerating neurons, as labeled by FluoroJade, was significantly decreased (33%) following HDAC1 expression (FIGS. 6E and 6G) Importantly, this demonstrates that reinforcement of HDAC1 activity can protect neurons against ischemia-induced DNA damage and neurotoxicity in vivo.

### HDAC and Neuronal Death

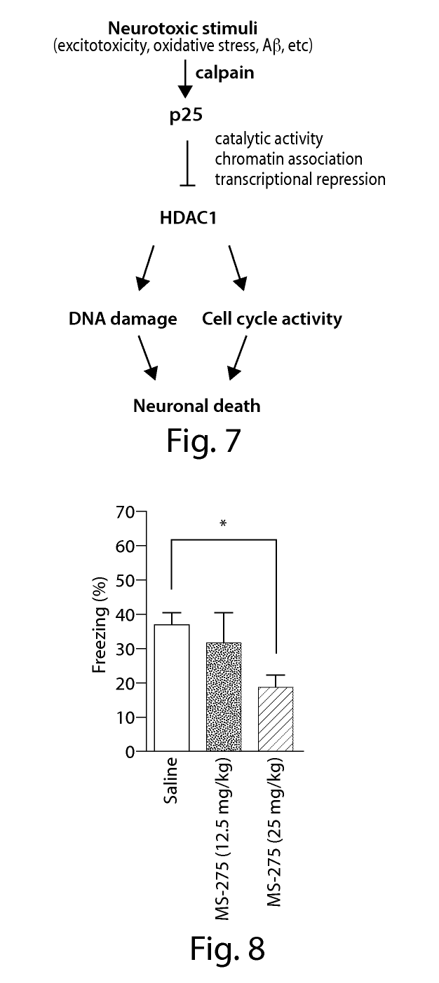

The CK-p25 mouse is a model for neurodegeneration in which neurons predictably to begin to die at around 5-6 weeks of induction (Cruz et al., 2003; Fischer et al., 2005). In our current study, using an unbiased approach of examining the gene expression profile at a specific time point of induction followed by validation, we determined that aberrant expression of cell cycle proteins and induction of double strand DNA breaks are early events in p25-mediated neurodegeneration. Furthermore, we identified deregulation of HDAC1 activity as a mechanism involved in p25-mediated DNA double strand break formation, cell cycle protein expression, and neuronal death. Collectively, our results outline a novel pathway in neurodegeneration by which the inactivation of HDAC1 by p25 leads to enhanced susceptibility of DNA to double strand breaks, and the de-repression of transcription leading to aberrant expression of cell cycle related genes. In addition, our findings provide mechanistic insights into a common link between DNA damage and aberrant cell cycle activity in neurodegeneration. As cell cycle reentry, DNA damage, and p25 accumulation are emerging as important pathological components of various neurodegenerative conditions, this mechanism may constitute a fundamental pathway in multiple neurodegenerative conditions involving neuronal loss including stroke/ischemia, Alzheimer's Disease, and Parkinson's Disease. The pathway is summarized in FIG. 7.

HDAC1 Inactivation by p25/cdk5

We have demonstrated that p25 can inhibit multiple aspects of HDAC1 activity, including HDAC1 catalytic activity and association of HDAC1 with chromatin. This inhibition appears to be cdk5 dependent (FIG. 4E). How does p25/cdk5 inhibit HDAC1? This may involve the posttranslational modification of HDAC1 by p25/cdk5. It was previously reported that HDAC1 catalytic activity and association with corepressors can be modulated by phosphorylation (Galasinski et al., 2002; Pflum et al., 2001). Alternatively, the p25/HDAC1 interaction may recruit p25/cdk5 to HDAC1-containing corepressor complexes, where p25/cdk5 phosphorylates and modulates co-repressors required for HDAC1 activity, such as mSin3a or SMRT/NcoR2 (de Ruijter et al., 2003; Nagy et al., 1997).

### HDAC1 Inactivation and Cell Cycle Reentry

While aberrant cell cycle activity in neurons in neurodegenerative states has been extensively documented, the underlying mechanisms and purposes are unclear. Our model introduces loss of HDAC1 activity as a novel underlying mechanism, and implies a simplified model of aberrant cell cycle activity as a chaotic transcriptional de-repression of multiple cell cycle genes that are normally suppressed in neurons. We have shown that p25/cdk5 inhibits the transcriptional repression activity of HDAC1 in a luciferase reporter system (FIG. 4E), and induces the disassociation of HDAC1 from the promotor region of cell cycle proteins E2F-1 and p21/WAF (FIG. 4G) Inhibition of HDAC1 in primary neurons resulted in upregulation of the cell cycle activity marker Ki-67 (FIG. 5B). Thus, our model implies that constitutive HDAC1, which is normally associated with and represses cell cycle related genes in postmitotic neurons, is inactivated by p25, leading to aberrant expression of cell cycle genes. The idea that aberrant cell cycle gene expression in neurons is a consequence of loss of HDAC1 repressional activity is consistent with the well known role of HDAC1 as a transcriptional repressor for many cell cycle genes including p21, E2F-1, and cyclins A and E (Brehm et al., 1998; Iavarone and Massague, 1999; Lagger et al., 2002; Rayman et al., 2002; Stadler et al., 2005; Stiegler et al., 1998).

It is also possible that the DNA damage induced by HDAC1 inactivation plays a role, as it has been demonstrated that increased oxidative DNA damage in ‘harlequin’ mouse mutants or drug-induced DNA damage in primary neurons can induce aberrant cell cycle activity (Klein et al., 2002; Kruman et al., 2004).

### HDAC1 Inactivation and DNA Damage

Double stranded DNA breaks were also observed to precede neuronal death in our p25 model. Our studies show that HDAC1 inactivation results in double strand DNA damage and cell cycle reentry, for instance through hypersensitization of chromatin to DNA damaging agents following loss of HDAC1 activity. In cancer cells, HDAC inhibitors can hypersensitize DNA to damaging agents such as UV and gamma-irradiation by increasing the acetylation state and thus the accessibility of chromatin (Cerra et al., 2006).

Interestingly, overexpression of p25 or HDAC1 inhibition or knockdown was sufficient to induce DNA damage in neurons and did not require additional genotoxic stimuli. Neurons are constantly subjected to DNA damaging events; for example, it has been estimated that the typical neuron of an aged mouse is subjected to 2,000,000 oxidative lesions per day (Hamilton et al., 2001). Therefore, enhanced accessibility to DNA damaging agents, combined with the relatively low levels of DNA repair factors present in neurons compared to proliferating cells (Gobbel et al., 1998; Nouspikel and Hanawalt, 2000, 2003), can result in an accumulation of DNA damage.

### DNA Damage, Cell Cycle Reentry, and Cell Death

In our current study, we report the formation of DNA double strand breaks in the CK-p25 model as well as in a rodent model for stroke/ischemia. Both DNA double strand breaks and cell cycle activity preceded and was later tightly associated with neurodegeneration (FIG. 3B). Compared to single nucleotide lesions such as 8-oxoguanine lesions, DNA double strand breaks are lethal lesions that induce cell cycle-dependent checkpoint responses in proliferating cells resulting in cell death (Sancar et al., 2004). However, because neurons are postmitotic, DNA damage events per se are postulated to have limited toxic consequences, with the exception of altered gene expression (Nouspikel and Hanawalt, 2003). Thus, DNA double strand breaks and cell cycle events such as DNA replication may synergistically induce cell death in CK-p25 neurons, likely in a checkpoint-dependent manner In support of this notion, the p53 DNA damage checkpoint protein is upregulated in the CK-p25 mice, and knockdown of p53 results in reduction of neuronal death in p25-transfected neurons (Kim et al., 2007).

### Role for HDAC1 in Postmitotic Neurons

As an important modulator of transcription, HDAC1 is undoubtedly involved in a variety of biological processes, and its involvement is well established in the regulation of the cell cycle in proliferating cells. Studies in the developing zebrafish retina demonstrate a role for HDAC1 in cell cycle exit and differentiation of retinal progenitors into neurons (Stadler et al., 2005; Yamaguchi et al., 2005). Our study implicates for the first time a crucial role for HDAC1 in the maintenance and survival of adult neurons as well. Our findings show a function for HDAC1 in maintaining a state of ‘quiescence’ through transcriptional repression of cell cycle genes. We also demonstrate a role for HDAC1 in maintaining DNA integrity in adult neurons, a function that may be tightly linked to its regulation of the accessibility of DNA to damaging agents. Collectively, our results outline an important role within the CNS for HDAC1, the deregulation of which can lead to aberrant expression of cell cycle genes, DNA damage, and ultimately death in adult neurons.

### Therapeutic Potential for HDAC1 Gain-of-Function

We have shown that inhibition of HDAC1 can lead to DNA damage, cell cycle gene expression, and neuronal death. In support of this finding, recent studies reporting the neuroprotective function of p130 and histone deacetylase-related protein (HDRP) demonstrated a requirement for association with HDAC1 for their pro-survival effects(Liu et al., 2005; Morrison et al., 2006). Furthermore, a recent phase I clinical trial of MS-275 in leukemia patients demonstrated neurologic toxicity manifesting as unsteady gait and somnolence as a dose-limiting toxicity (DLT)(Gojo et al., 2006).

On the other hand, it is clear that HDAC inhibitors have beneficial effects. We recently demonstrated that treatment with the nonselective HDAC inhibitor sodium butyrate enhanced synapse formation and long term memory recall. Along similar lines, studies have shown beneficial effects of HDAC inhibitors in patients or models of psychiatric disorders such as depression (Citrome, 2003; Johannessen and Johannessen, 2003; Tsankova et al., 2006). In addition, HDAC inhibitors such as phenylbutyrate had neuroprotective properties, within a therapeutic window, in models of Huntington's disease (HD)(Hockly et al., 2003; Langley et al., 2005; McCampbell et al., 2001; Steffan et al., 2001). The use of HDAC inhibitors in HD models is based on the finding that Huntingtin inhibits the histone acetyltransferases CREB-binding protein (CBP) and p300/CBP associated factor (P/CAF), leading to a deficiency in levels of histone acetylation (Bates, 2001).

Thus, it is evident that both beneficial and adverse signals can be triggered by histone deacetylase inhibition. Which signals are triggered is likely to be dependent on the specific genes and HDAC members that are affected. For example, while nonselective HDAC inhibitors improved contextual fear conditioning-based learning (Fischer et al., 2007; Levenson et al., 2004), treatment with the class I-specific inhibitor MS-275 inhibited learning (FIG. 8) and induced massive DNA damage (FIG. 5C). Furthermore, treatment with the non-selective HDAC inhibitor SAHA (suberoylanilide hydroxamic acid) at submicromolar concentrations, but not MS-275, induced expression of the synaptic plasticity-associated gene brain-derived neurotrophic factor (BDNF) in a glioma cell line (C6) (data not shown). It was recently shown that specific downregulation of the class II HDACs HDAC4 and HDAC5 by the antidepressant imipramine de-repressed BDNF expression and suppressed depression-like behavior (Tsankova et al., 2006). Thus, de-repression of HDAC class II-repressed synaptic plasticity genes such as BDNF can elicit beneficial responses, while de-repression of HDAC1-repressed cell cycle genes can have deleterious consequences. Beneficial versus deleterious effects of HDAC inhibition may also closely depend on the dosage and/or length of HDAC inhibition. For example, numerous studies have demonstrated neurotoxic effects of high dose HDAC inhibitor treatment (Boutillier et al., 2002, 2003; Kim et al., 2004; Salminen et al., 1998).

Our current study demonstrates for the first time the therapeutic potential for replenishing HDAC1 activity in certain neurodegenerative contexts such as ischemia (FIG. 6). The previous studies with HDAC inhibitors and our current study, collectively, illustrate the complex and broadly impacting nature of manipulating HDAC activity, and underline the importance of chromatin regulation in a variety of processes in the CNS. Importantly, our study exemplifies the catastrophic consequences of deregulation of this process, and introduces a novel and unexpected avenue for therapeutic strategies in neurodegeneration.

### Experiment 8: Identification of HDAC Activators

To identify small molecule activators of HDAC1, a diverse collection of 1,760 small molecules composed of synthetic compounds, natural products, and a subset of FDA approved drugs were arrayed in 384-well plates as ˜10 mM dimethylsulphoxide (DMSO) stocks. To identify modulators (both activators and inhibitors) of HDACs, a fluorescence-based assay that utilizes Caliper's mobility shift assay technology (Hopkinton, Mass.) was used. This assay is based on the electrophoretic separation of N-acetyl lysine peptide substrate from the deacetylated product, which bears an additional positive charge. By allowing direct visualization of fluorophore-labeled separated substrate and product, this assay minimizes interference from fluorescent compounds during screening and does not require the use of coupling enzymes. The product and substrate in each independent reaction were separated using a microfluidic chip (Caliper Life Sciences) run on a Caliper LC3000 (Caliper Life Sciences). The product and substrate fluorophore were excited at 488 nm and detected at 530 nm. Substrate conversion was calculated from the electrophoregram using HTS Well Analyzer software (Caliper Life Sciences). Since the amount of converted substrate is measured, and the reactions were performed at the Kₘ for each enzyme, it is possible to identify both inhibitors and activators of HDACs using this assay.

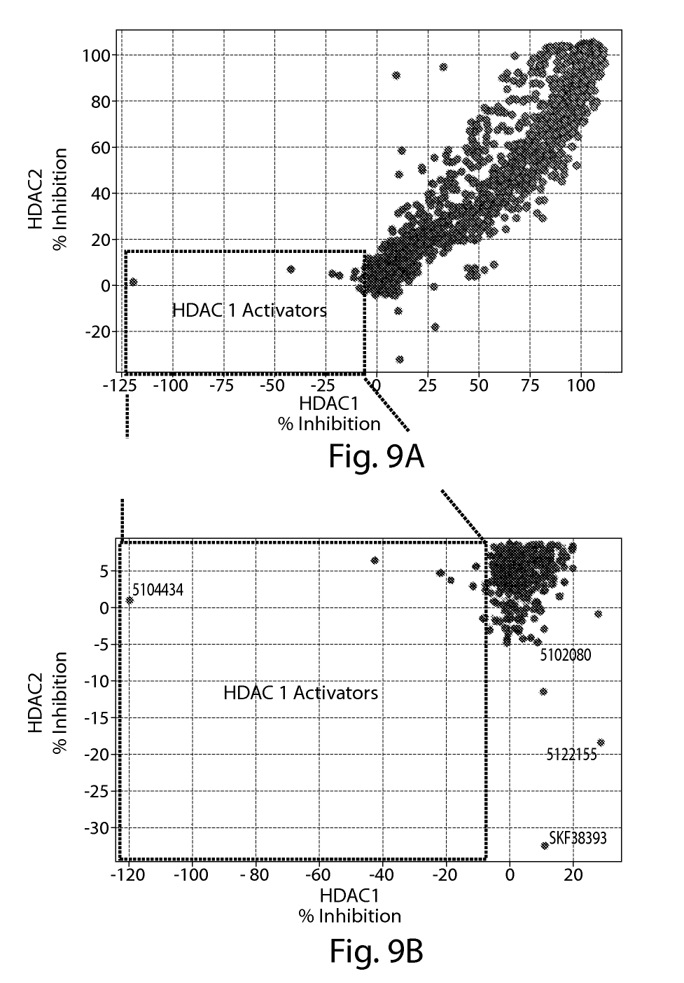

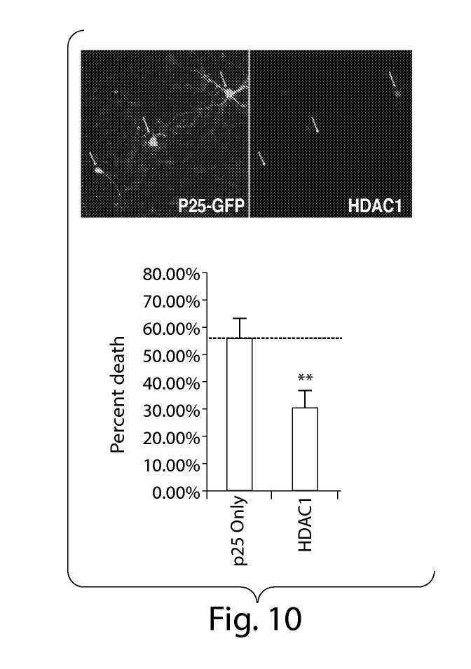

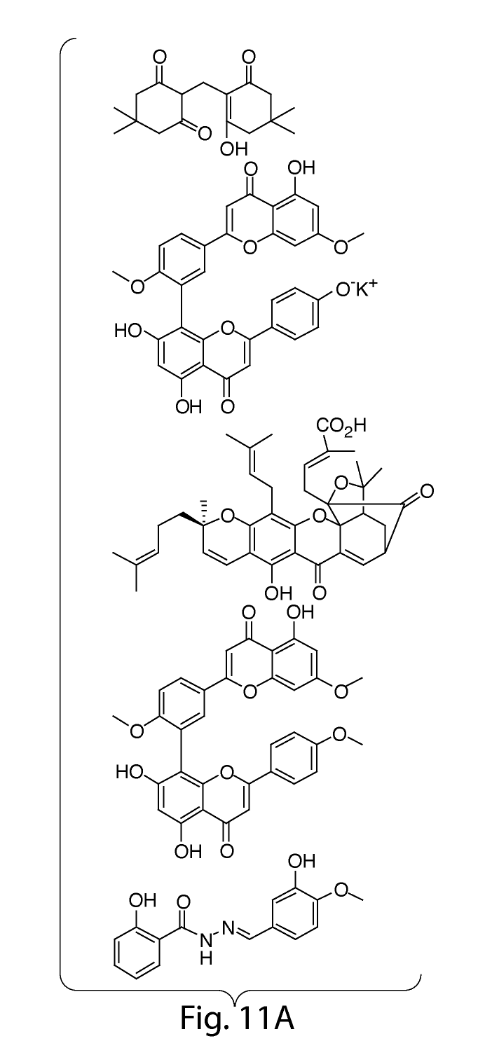

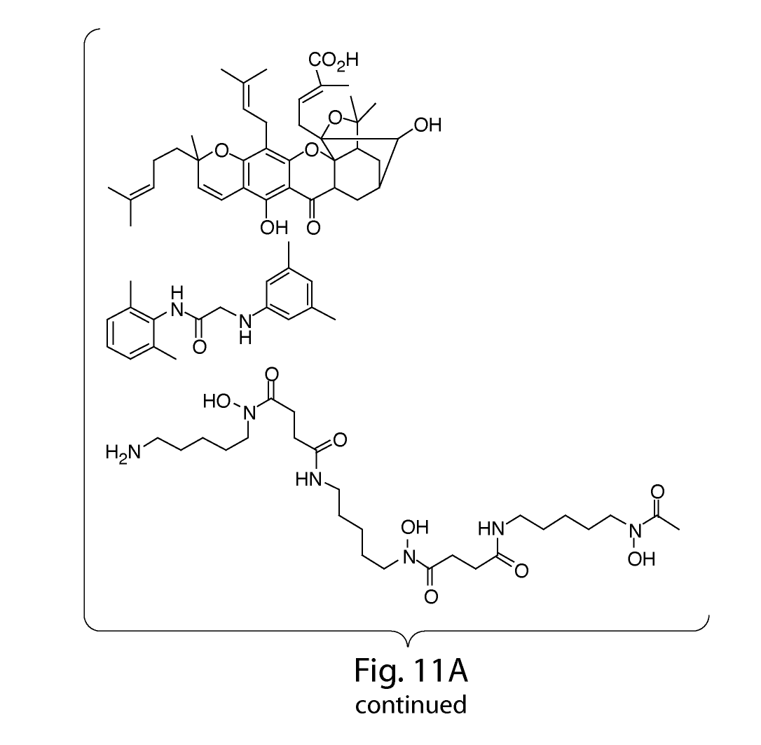

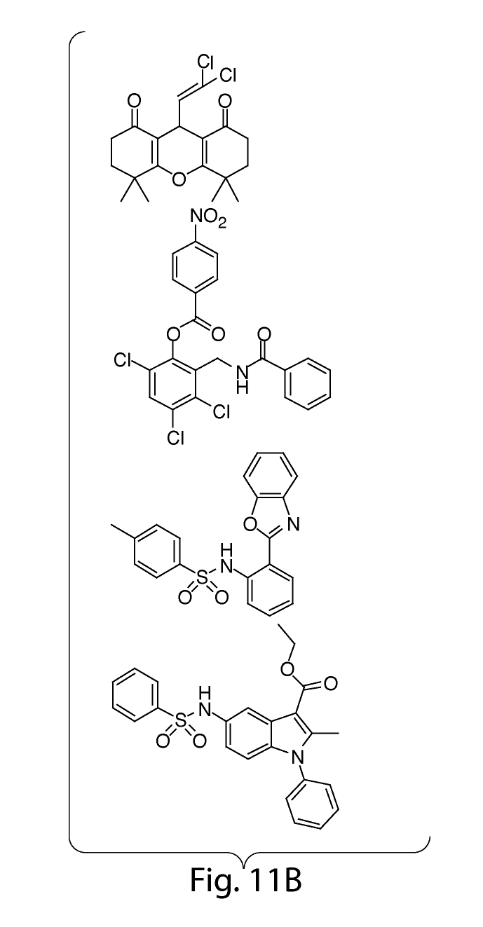

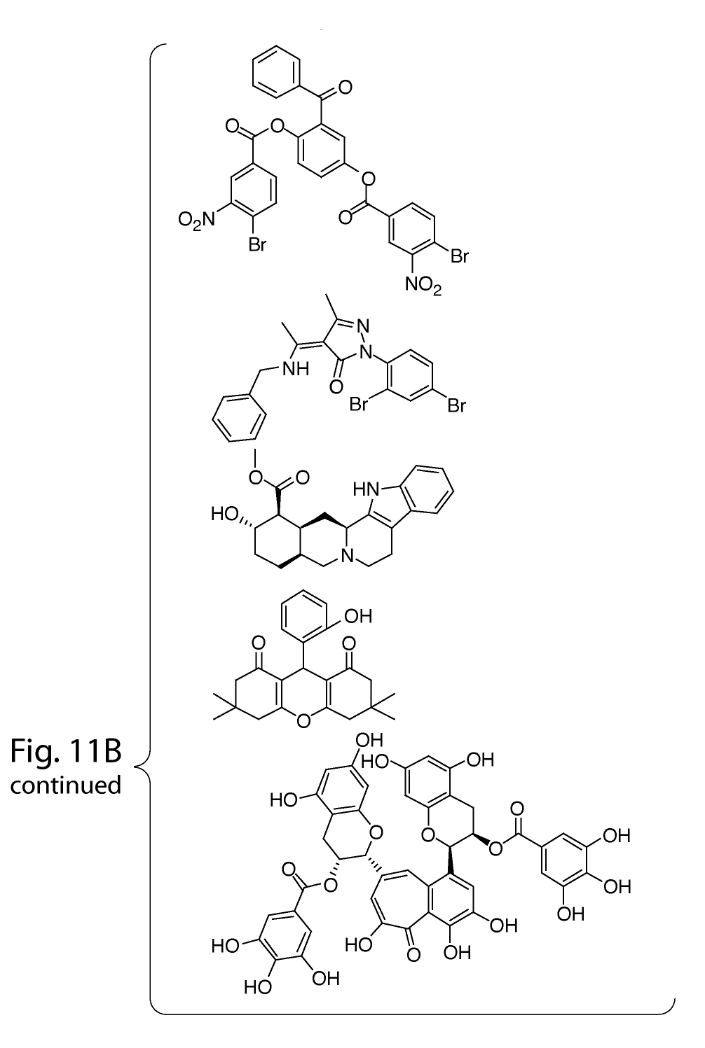

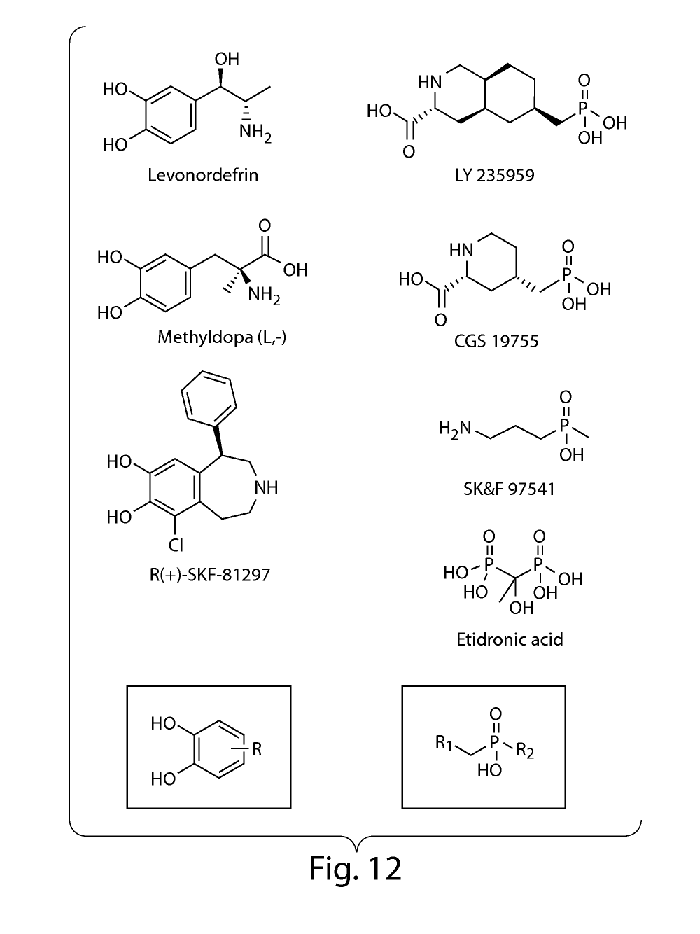

Using the mobility shift assay, all compounds were screened in duplicate using a panel of class I and class II HDACs and a N-acetyl lysine peptide substrate. For class I HDACs, HDAC1, HDAC2, and HDAC8 were used. For class IIb HDACs, HDAC6 and to HDAC10 were used. Compounds were incubated for 18-24 hrs and the percent inhibition (avg. n=2) relative to a solvent (DMSO) control treatment of each compound determined through measurement of substrate conversion. As shown in FIG. 9A, while most compounds in the library were inhibitors of the deacetylase activity of HDAC1 and HDAC2, a small percentage of compounds, shown highlighted in FIG. 9B, were found to be activators, which in the assay corresponds to negative inhibition. For example, cpd-5104434 was found to activate HDAC1 ˜120%, while having no effect on HDAC2. Table 4 provides a summary of the top HDAC1 activators and selectivity profile against class I and class II HDAC. FIG. 11 provides a list of all of the structures that activated HDAC1 by a value of 5% or greater.

TABLE 4
HDAC1 activators and selectivity profile against Class I and Class II HDACs.

| Compound Name | Class | Source | Vendor ID | MolWt | Conc. (μM) | HDAC1 | HDAC2 | HDAC8 | HDAC10 | HDAC6 |
|---|---|---|---|---|---|---|---|---|---|---|
| 510443d | synthetic | ChemBridge | 5104434 | 292.37 | 19 | 120 | −1 | −6 | nd | −24 |
| genxgetin K | natural product | MicroSource | 200436 | 566.51 | 20 | 42 | −7 | −3 | 56 | −18 |
| gambogic acid | natural product | Biomol | AP305 | 628.75 | 18 | 22 | −5 | −8 | 13 | −63 |
| sciadopitysin | natural product | indofine | 210218 | 680.54 | 19 | 19 | −4 | 0 | 19 | −5 |
| 5193892 | synthetic | ChemBridge | 5193892 | 286.28 | 39 | 12 | −3 | −1 | −4 | −29 |
| tetrahydrogamboic | natural product | Gsis | G1070 | 632.78 | 18 | 11 | −6 | −1 | 13 | −10 |
| acid | | | | | | | | | | |
| TAM-11 | synthetic | ChemBridge | 5213008 | 282.38 | 20 | 9 | 1 | 3 | 9 | −4 |
| deferoxamine | FDA approved | Sigma | D9533 | 560.68 | 40 | 8 | −3 | 4 | 2 | −5 |
| | drug | | | | | | | | | |
| TAM-13 | synthetic | ChemBridge | 5151277 | 359.28 | 14 | 6 | 3 | 4 | 2 | 22 |
| TAM-7 | synthetic | ChemBridge | 5114445 | 479.7 | 11 | 5 | −7 | 0 | 1 | −6 |
| TAM-8 | synthetic | ChemBridge | 5252917 | 364.42 | 16 | 5 | −4 | 0 | 0 | −8 |
| 5100018 | synthetic | ChemBridge | 5100018 | 434.51 | 26 | 5 | −6 | 0 | −5 | −2 |
| TAM-9 | synthetic | ChemBridge | 5162773 | 670.22 | 8 | 5 | −6 | −1 | −6 | −10 |
| TAM-12 | synthetic | ChemBridge | 5248896 | 466.17 | 11 | 5 | −1 | 2 | −3 | −4 |
| alpha-yohimbine | natural product | Biomol | AR106 | 354.44 | 31 | 5 | −5 | 0 | 1 | −2 |
| 5213720 | synthetic | ChemBridge | 5213720 | 366.45 | 15 | 5 | −4 | 0 | −4 | −6 |
| theaflavin | natural product | MicroSource | 200111 | 868.7 | 12 | 5 | −8 | −10 | 0 | −79 |

Values indicate % activation (avg. n = 2) of deacetylase activity at the indicated concentration measured using recombinant human HDACs assayed with Caliper's mobility shift assay technology.

### HDAC Activators

We identified a variety of HDAC activators. Three classes of compounds are highlighted below.

Type I Approved Drugs. One active HDAC1 modulators (8% activation), is the iron chelator deferoxamine, which is an FDA approved drug that is used to treat acute iron poisoning. This compound has also been shown to be efficacious in ameliorating hypoxic-ischemic brain injury. Deferoxamine, and other iron chelators enhance the activity of to HDAC1.
Type II. Natural Products. Two HDAC1 activators are flavonoids, which are naturally occurring polyphenolic compounds present in a variety of fruits, vegetables, and seeds, which have many biological properties, including antioxidative and anti-inflammatory properties. Flavonoids can be classified into flavanones, flavones, flavonols, and biflavones. The latter class of biflavonoids consist of a dimer of flavonoids linked to each other by either a C—C or a C—O—C covalent bond. The results described herein imply that flavonoids, such as the biflavonoid ginkgetin K isolated from Ginkgo biloba, have therapeutic potential against neurological disorders, including ischemic stroke and Alzheimer's disease, through the activation of HDAC1.
Type III Synthetic Compounds. A number of the HDAC1 activators (labeled TAM in Table 1) were identified in a cell-based assay looking for “suppressors” of the HDAC inhibitor (trichostatin A). The compounds may target HDACs directly and increasing their deacetylase activity.

### Experiment 9: HDAC Activator Biochemical Assays

The in vitro activities of recombinant human HDACs 1,2,3 and 5 (BPS Biosciences), as summarized in Table 5, were measured with a 384-well plate based fluorometric deacetylase assay making use of acetylated tripeptide substrates that are amide-coupled to 7-amino-4-methylcoumarin that can detect either Class I/IIb (substrate MAZ1600) or Class IIa/HDAC8 (substrate MAZ1675) HDAC activity as described in detail in Bradner et al. (2009), with the following modifications: HDAC1 (4.5 ng/reaction; MAZ1600 Kₘ=6 μM); HDAC2 (4 ng/reaction; MAZ1600 Kₘ=4.5 μM); HDAC3 (2 ng/reaction; MAZ1600 Kₘ=9.5 μM) and HDACS (1 ng/reaction; MAZ1675 Kₘ=57 μM). TCEP was omitted from the assay buffer. Rates of reactions (slopes) were normalized to the mean of DMSO control treatments for each enzyme on each plate. Bradner J E, West N, Grachan M L, Greenberg E F, Haggarty S J, Mazitsheck. Nature Chemical Biology (under review). Bradner J E, West N, Grachan M L, Greenberg E F, Haggarty S J, Mazitsheck. Chemical Phylogenetics of Histone Deacetylases. Nature Chemical Biology 2009. Z

TABLE 5
Results of HDAC Activator Biochemical Assays

| | Compound | | HDAC1 | HDAC2 | HDAC3 | HDAC5 | % HDAC1 | % HDAC2 | % HDAC3 | % HDAC5 |
|---|---|---|---|---|---|---|---|---|---|---|
| Classification | Name | Structure | slope | slope | slope | slope | Activ | Activ | Activ | activ |
| Control | DMSO | | 37787 | 40839 | 54625 | 50401 | 1.00 | 1.00 | 1.00 | 1.00 |
| HDAC1 & HDAC3 activator | Ampicillin trihydrate | 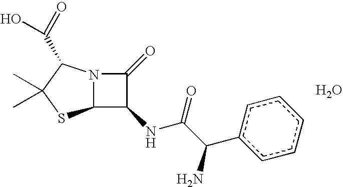 | 40160 | 43966 | 56660 | 46606 | 1.06 | 1.08 | 1.04 | 0.92 |
| HDAC1 & HDAC3 activator | Etidronic acid, disodium salt | 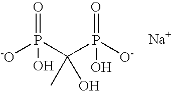 | 40241 | 41315 | 58286 | 51711 | 1.06 | 1.01 | 1.07 | 1.03 |
| HDAC1 & HDAC3 activator | Levonordefrin | 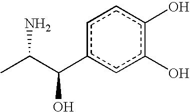 | 40405 | 40182 | 62457 | 47752 | 1.07 | 0.98 | 1014 | 0.95 |
| HDAC1 & HDAC3 activator | LY 235959 | 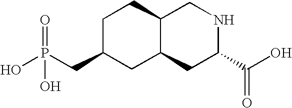 | 40893 | 42923 | 65435 | 43688 | 1.08 | 1.05 | 1.20 | 0.87 |
| HDAC1 & HDAC3 activator | Methyldopa (L,-) | 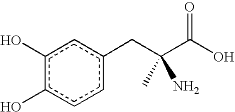 | 40553 | 42153 | 60891 | 56516 | 1.07 | 1.03 | 1.11 | 1.12 |
| HDAC1 & HDAC3 activator | Oxalamine citrate salt | 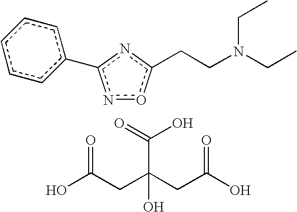 | 40700 | 41269 | 60752 | 47216 | 1.08 | 1.01 | 1.11 | 0.94 |
| HDAC1 & HDAC3 activator | R(+)-SKF- 81297 | 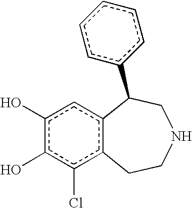 | 40201 | 42197 | 66733 | 48294 | 1.06 | 1.03 | 1.22 | 0.96 |
| HDAC1 activator | (+.-)-4- AMINO-3- (5- CHLORO- 2- THIENYL)- BUTANOIC ACID | 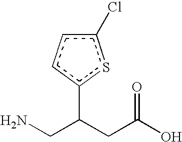 | 40239 | 40008 | 54049 | 50272 | 1.06 | 0.98 | 0.99 | 1.00 |
| HDAC1 activator | (RS)- (TETRAZOL- 5- YL)GLYCINE | 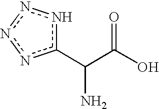 | 40343 | 42215 | 56930 | 45589 | 1.07 | 1.03 | 1.04 | 0.90 |
| HDAC1 activator | CGS 19755 | 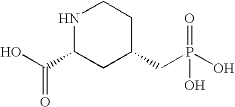 | 41839 | 42301 | 57057 | 48280 | 1.11 | 1.04 | 1.04 | 0.96 |
| HDAC1 activator | D- ASPARTIC ACID | 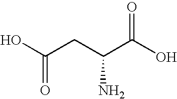 | 40655 | 42016 | 54899 | 45291 | 1.08 | 1.03 | 1.01 | 0.90 |
| HDAC1 activator | gamma-D- GLUTAMYL- AMINO METHYL- SULFONIC ACID | 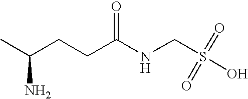 | 39984 | 42116 | 54643 | 42078 | 1.06 | 1.03 | 1.00 | 0.83 |
| HDAC1 activator | Phenazo- pyridine hydrochloride | 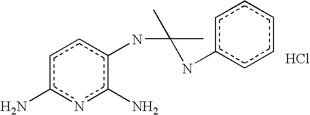 | 40631 | 42470 | 56613 | 54125 | 1.08 | 1.04 | 1.04 | 1.07 |
| HDAC1 activator | Podophyllo toxin | 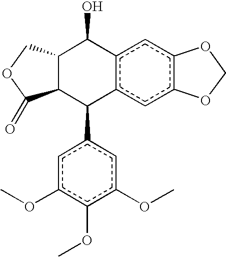 | 40983 | 39197 | 53416 | 54734 | 1.08 | 0.96 | 0.98 | 1.09 |
| HDAC1 activator | SK&F 97541 | 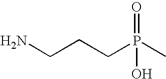 | 40213 | 39881 | 54915 | 49250 | 1.06 | 0.98 | 1.01 | 0.98 |

### REFERENCES

Adamec, E., Vonsattel, J. P., and Nixon, R. A. (1999). DNA strand breaks in Alzheimer's disease. Brain Res 849, 67-77.

al-Ubaidi, M. R., Hollyfield, J. G., Overbeek, P. A., and Baehr, W. (1992). Photoreceptor degeneration induced by the expression of simian virus 40 large tumor antigen in the retina of transgenic mice. Proc Natl Acad Sci U S A 89, 1194-1198.

Andegeko, Y., Moyal, L., Mittelman, L., Tsarfaty, I., Shiloh, Y., and Rotman, G. (2001). Nuclear retention of ATM at sites of DNA double strand breaks. J Biol Chem 276, 38224-38230.

Andorfer, C., Acker, C. M., Kress, Y., Hof, P. R., Duff, K., and Davies, P. (2005). Cell-cycle reentry and cell death in transgenic mice expressing nonmutant human tau isoforms. J Neurosci 25, 5446-5454.

Bates, G. P. (2001). Huntington's disease. Exploiting expression. Nature 413, 691, 693-694.

Boutillier, A. L., Trinh, E., and Loeffler, J. P. (2002). Constitutive repression of E2F1 transcriptional activity through HDAC proteins is essential for neuronal survival. Ann N Y Acad Sci 973, 438-442.

Boutillier, A. L., Trinh, E., and Loeffler, J. P. (2003). Selective E2F-dependent gene transcription is controlled by histone deacetylase activity during neuronal apoptosis. J Neurochem 84, 814-828.

Brehm, A., Miska, E. A., McCance, D. J., Reid, J. L., Bannister, A. J., and Kouzarides, T. (1998). Retinoblastoma protein recruits histone deacetylase to repress transcription. Nature 391, 597-601.

Busser, J., Geldmacher, D. S., and Herrup, K. (1998). Ectopic cell cycle proteins predict the sites of neuronal cell death in Alzheimer's disease brain. J Neurosci 18, 2801-2807.

Cerna, D., Camphausen, K., and Tofilon, P. J. (2006). Histone deacetylation as a target for radiosensitization. Curr Top Dev Biol 73, 173-204.

Citrome, L. (2003). Schizophrenia and valproate. Psychopharmacol Bull 37 Suppl 2, 74-88.

Cruz, J. C., Tseng, H. C., Goldman, J. A., Shih, H., and Tsai, L. H. (2003). Aberrant Cdk5 activation by p25 triggers pathological events leading to neurodegeneration and neurofibrillary tangles. Neuron 40, 471-483.

de Ruijter, A. J., van Gennip, A. H., Caron, H. N., Kemp, S., and van Kuilenburg, A. B. (2003). Histone deacetylases (HDACs): characterization of the classical HDAC family. Biochem J 370, 737-749.

Dhavan, R., and Tsai, L. H. (2001). A decade of CDKS. Nat Rev Mol Cell Biol 2, 749-759.

Dhawan, A., Mathur, N., and Seth, P. K. (2001). The effect of smoking and eating habits on DNA damage in Indian population as measured in the Comet assay. Mutat Res 474, 121-128.

Ferrante, R. J., Browne, S. E., Shinobu, L. A., Bowling, A. C., Baik, M. J., MacGarvey, U., Kowall, N. W., Brown, R. H., Jr., and Beal, M. F. (1997). Evidence of increased oxidative damage in both sporadic and familial amyotrophic lateral sclerosis. J Neurochem 69, 2064-2074.

Fischer, A., Sananbenesi, F., Pang, P. T., Lu, B., and Tsai, L. H. (2005). Opposing roles of transient and prolonged expression of p25 in synaptic plasticity and hippocampus-dependent memory. Neuron 48, 825-838.

Fischer, A., Sananbenesi, F., Wang, X., Dobbin, M., and Tsai, L. H. (2007). Recovery of learning and memory is associated with chromatin remodelling. Nature 447, 178-182.

Galasinski, S. C., Resing, K. A., Goodrich, J. A., and Ahn, N. G. (2002). Phosphatase inhibition leads to histone deacetylases 1 and 2 phosphorylation and disruption of corepressor interactions. J Biol Chem 277, 19618-19626.

Garcia-Bonilla, L., Burda, J., Pineiro, D., Ayuso, I., Gomez-Calcerrada, M., and Salinas, M. (2006). Calpain-induced proteolysis after transient global cerebral ischemia and ischemic tolerance in a rat model. Neurochem Res 31, 1433-1441.

Gobbel, G. T., Bellinzona, M., Vogt, A. R., Gupta, N., Fike, J. R., and Chan, P. H. (1998). Response of postmitotic neurons to X-irradiation: implications for the role of DNA damage in neuronal apoptosis. J Neurosci 18, 147-155.

Gojo, I., Jiemjit, A., Trepel, J. B., Sparreboom, A., Figg, W. D., Rollins, S., Tidwell, M. L., Greer, J., Chung, E. J., Lee, M. J., et al. (2006). Phase 1 and pharmacological study of MS-275, a histone deacetylase inhibitor, in adults with refractory and relapsed acute leukemias. Blood.

Gui, C. Y., Ngo, L., Xu, W. S., Richon, V. M., and Marks, P. A. (2004). Histone deacetylase (HDAC) inhibitor activation of p21WAF1 involves changes in promoter-associated proteins, including HDAC1. Proc Natl Acad Sci U S A 101, 1241-1246.

Hamilton, M. L., Van Remmen, H., Drake, J. A., Yang, H., Guo, Z. M., Kewitt, K., Walter, C. A., and Richardson, A. (2001). Does oxidative damage to DNA increase with age? Proc Natl Acad Sci U S A 98, 10469-10474.

Hayashi, T., Sakai, K., Sasaki, C., Zhang, W. R., and Abe, K. (2000). Phosphorylation of retinoblastoma protein in rat brain after transient middle cerebral artery occlusion. Neuropathol Appl Neurobiol 26, 390-397.

Hayashi, T., Sakurai, M., Itoyama, Y., and Abe, K. (1999). Oxidative damage and breakage of DNA in rat brain after transient MCA occlusion. Brain Res 832, 159-163.

Herrup, K., and Busser, J. C. (1995). The induction of multiple cell cycle events precedes target-related neuronal death. Development 121, 2385-2395.

Hockly, E., Richon, V. M., Woodman, B., Smith, D. L., Zhou, X., Rosa, E., Sathasivam, K., Ghazi-Noori, S., Mahal, A., Lowden, P. A., et al. (2003). Suberoylanilide hydroxamic acid, a histone deacetylase inhibitor, ameliorates motor deficits in a mouse model of Huntington's disease. Proc Natl Acad Sci U S A 100, 2041-2046.

Hu, E., Dul, E., Sung, C. M., Chen, Z., Kirkpatrick, R., Zhang, G. F., Johanson, K., Liu, R., Lago, A., Hofmann, G., et al. (2003). Identification of novel isoform-selective inhibitors within class I histone deacetylases. J Pharmacol Exp Ther 307, 720-728.

Iavarone, A., and Massague, J. (1999). E2F and histone deacetylase mediate transforming growth factor beta repression of cdc25A during keratinocyte cell cycle arrest. Mol Cell Biol 19, 916-922.

Ishizuka, T., and Lazar, M. A. (2003). The N-CoR/histone deacetylase 3 complex is required for repression by thyroid hormone receptor. Mol Cell Biol 23, 5122-5131.

Johannessen, C. U., and Johannessen, S. I. (2003). Valproate: past, present, and future. CNS Drug Rev 9, 199-216.

Kim, D., Nguyen, M. D., Dobbin, M. M., Fischer, A., Sananbenesi, F., Rodgers, J. T., Delalle, I., Baur, J. A., Sui, G., Armour, S. M., et al. (2007). SIRT1 deacetylase protects against neurodegeneration in models for Alzheimer's disease and amyotrophic lateral sclerosis. Embo J 26, 3169-3179.

Kim, H. S., Kim, E. M., Kim, N. J., Chang, K. A., Choi, Y., Ahn, K. W., Lee, J. H., Kim, S., Park, C. H., and Suh, Y. H. (2004) Inhibition of histone deacetylation enhances the neurotoxicity induced by the C-terminal fragments of amyloid precursor protein. J Neurosci Res 75, 117-124.

Klein, J. A., Longo-Guess, C. M., Rossmann, M. P., Seburn, K. L., Hurd, R. E., Frankel, W. N., Bronson, R. T., and Ackerman, S. L. (2002). The harlequin mouse mutation downregulates apoptosis-inducing factor. Nature 419, 367-374.

Konishi, Y., and Bonni, A. (2003). The E2F-Cdc2 cell-cycle pathway specifically mediates activity deprivation-induced apoptosis of postmitotic neurons. J Neurosci 23, 1649-1658.

Konishi, Y., Lehtinen, M., Donovan, N., and Bonni, A. (2002). Cdc2 phosphorylation of BAD links the cell cycle to the cell death machinery. Mol Cell 9, 1005-1016.

Kruman, II, Wersto, R. P., Cardozo-Pelaez, F., Smilenov, L., Chan, S. L., Chrest, F. J., Emokpae, R., Jr., Gorospe, M., and Mattson, M. P. (2004). Cell cycle activation linked to neuronal cell death initiated by DNA damage. Neuron 41, 549-561.

Lagger, G., O'Carroll, D., Rembold, M., Khier, H., Tischler, J., Weitzer, G., Schuettengruber, B., Hauser, C., Brunmeir, R., Jenuwein, T., and Seiser, C. (2002). Essential function of histone deacetylase 1 in proliferation control and CDK inhibitor repression. Embo J 21, 2672-2681.

Langley, B., Gensert, J. M., Beal, M. F., and Ratan, R. R. (2005). Remodeling chromatin and stress resistance in the central nervous system: histone deacetylase inhibitors as novel and broadly effective neuroprotective agents. Curr Drug Targets CNS Neurol Disord 4, 41-50.

Lee, M. S., Kwon, Y. T., Li, M., Peng, J., Friedlander, R. M., and Tsai, L. H. (2000). Neurotoxicity induces cleavage of p35 to p25 by calpain. Nature 405, 360-364.

Levenson, J. M., O′Riordan, K. J., Brown, K. D., Trinh, M. A., Molfese, D. L., and Sweatt, J. D. (2004). Regulation of histone acetylation during memory formation in the hippocampus. J Biol Chem 279, 40545-40559.

Liu, D. X., and Greene, L. A. (2001). Regulation of neuronal survival and death by E2F-dependent gene repression and derepression. Neuron 32, 425-438.

Liu, D. X., Nath, N., Chellappan, S. P., and Greene, L. A. (2005). Regulation of neuron survival and death by p130 and associated chromatin modifiers. Genes Dev 19, 719-732.

Lu, T., Pan, Y., Kao, S. Y., Li, C., Kohane, I., Chan, J., and Yankner, B. A. (2004). Gene regulation and DNA damage in the ageing human brain. Nature 429, 883-891.

McCampbell, A., Taye, A. A., Whitty, L., Penney, E., Steffan, J. S., and Fischbeck, K. H. (2001). Histone deacetylase inhibitors reduce polyglutamine toxicity. Proc Natl Acad Sci U S A 98, 15179-15184.

McShea, A., Lee, H. G., Petersen, R. B., Casadesus, G., Vincent, I., Linford, N. J., Funk, J. O., Shapiro, R. A., and Smith, M. A. (2006). Neuronal cell cycle re-entry mediates Alzheimer disease-type changes. Biochim Biophys Acta.

Mitsios, N., Pennucci, R., Krupinski, J., Sanfeliu, C., Gaffney, J., Kumar, P., Kumar, S., Juan-Babot, O., and Slevin, M. (2007). Expression of cyclin-dependent kinase 5 mRNA and protein in the human brain following acute ischemic stroke. Brain Pathol 17, 11-23.

Morrison, B. E., Majdzadeh, N., Zhang, X., Lyles, A., Bassel-Duby, R., Olson, E. N., and D'Mello, S. R. (2006). Neuroprotection by histone deacetylase-related protein. Mol Cell Biol 26, 3550-3564.

Nagy, L., Kao, H. Y., Chakravarti, D., Lin, R. J., Hassig, C. A., Ayer, D. E., Schreiber, S. L., and Evans, R. M. (1997). Nuclear receptor repression mediated by a complex containing SMRT, mSin3A, and histone deacetylase. Cell 89, 373-380.

Nguyen, M. D., Lariviere, R. C., and Julien, J. P. (2001). Deregulation of Cdk5 in a mouse model of ALS: toxicity alleviated by perikaryal neurofilament inclusions. Neuron 30, 135-147.

Nguyen, M. D., Mushynski, W. E., and Julien, J. P. (2002). Cycling at the interface between neurodevelopment and neurodegeneration. Cell Death Differ 9, 1294-1306.

Nouspikel, T., and Hanawalt, P. C. (2000). Terminally differentiated human neurons repair transcribed genes but display attenuated global DNA repair and modulation of repair gene expression. Mol Cell Biol 20, 1562-1570.

Nouspikel, T., and Hanawalt, P. C. (2003). When parsimony backfires: neglecting DNA repair may doom neurons in Alzheimer's disease. Bioessays 25, 168-173.

Padmanabhan, J., Park, D. S., Greene, L. A., and Shelanski, M. L. (1999). Role of cell cycle regulatory proteins in cerebellar granule neuron apoptosis. J Neurosci 19, 8747-8756.

Patrick, G. N., Zukerberg, L., Nikolic, M., de la Monte, S., Dikkes, P., and Tsai, L. H. (1999). Conversion of p35 to p25 deregulates Cdk5 activity and promotes neurodegeneration. Nature 402, 615-622.

Peng, P. L., Zhong, X., Tu, W., Soundarapandian, M. M., Molner, P., Zhu, D., Lau, L., Liu, S., Liu, F., and Lu, Y. (2006). ADAR2-dependent RNA editing of AMPA receptor subunit GluR2 determines vulnerability of neurons in forebrain ischemia. Neuron 49, 719-733.

Pflum, M. K., Tong, J. K., Lane, W. S., and Schreiber, S. L. (2001). Histone deacetylase 1 phosphorylation promotes enzymatic activity and complex formation. J Biol Chem 276, 47733-47741.

Rashidian, J., Iyirhiaro, G. O., and Park, D. S. (2007). Cell cycle machinery and stroke. Biochim Biophys Acta 1772, 484-493.

Rayman, J. B., Takahashi, Y., Indjeian, V. B., Dannenberg, J. H., Catchpole, S., Watson, R. J., to Riele, H., and Dynlacht, B. D. (2002). E2F mediates cell cycle-dependent transcriptional repression in vivo by recruitment of an HDAC1/mSin3B corepressor complex. Genes Dev 16, 933-947.

Robison, S. H., and Bradley, W. G. (1984). DNA damage and chronic neuronal degenerations. J Neurol Sci 64, 11-20.

Rolig, R. L., and McKinnon, P. J. (2000) Linking DNA damage and neurodegeneration. Trends Neurosci 23, 417-424.

Roth, S. Y., Denu, J. M., and Allis, C. D. (2001). Histone acetyltransferases. Annu Rev Biochem 70, 81-120.

Salminen, A., Tapiola, T., Korhonen, P., and Suuronen, T. (1998). Neuronal apoptosis induced by histone deacetylase inhibitors. Brain Res Mol Brain Res 61, 203-206.

Sancar, A., Lindsey-Boltz, L. A., Unsal-Kacmaz, K., and Linn, S. (2004). Molecular mechanisms of mammalian DNA repair and the DNA damage checkpoints. Annu Rev Biochem 73, 39-85.

Smith, P. D., Crocker, S. J., Jackson-Lewis, V., Jordan-Sciutto, K. L., Hayley, S., Mount, M. P., O′Hare, M. J., Callaghan, S., Slack, R. S., Przedborski, S., et al. (2003). Cyclin-dependent kinase 5 is a mediator of dopaminergic neuron loss in a mouse model of Parkinson's disease. Proc Natl Acad Sci U S A 100, 13650-13655.

Stadler, J. A., Shkumatava, A., Norton, W. H., Rau, M. J., Geisler, R., Fischer, S., and Neumann, C. J. (2005). Histone deacetylase 1 is required for cell cycle exit and differentiation in the zebrafish retina. Dev Dyn 233, 883-889.

Steffan, J. S., Bodai, L., Pallos, J., Poelman, M., McCampbell, A., Apostol, B. L., Kazantsev, A., Schmidt, E., Zhu, Y. Z., Greenwald, M., et al. (2001). Histone deacetylase inhibitors arrest polyglutamine-dependent neurodegeneration in Drosophila. Nature 413, 739-743.

Stiegler, P., De Luca, A., Bagella, L., and Giordano, A. (1998). The COOH-terminal region of pRb2/p130 binds to histone deacetylase 1 (HDAC1), enhancing transcriptional repression of the E2F-dependent cyclin A promoter. Cancer Res 58, 5049-5052.

Swatton, J. E., Sellers, L. A., Faull, R. L., Holland, A., Iritani, S., and Bahn, S. (2004). Increased MAP kinase activity in Alzheimer's and Down syndrome but not in schizophrenia human brain. Eur J Neurosci 19, 2711-2719.

Taunton, J., Hassig, C. A., and Schreiber, S. L. (1996). A mammalian histone deacetylase related to the yeast transcriptional regulator Rpd3p. Science 272, 408-411.

Tsankova, N. M., Berton, O., Renthal, W., Kumar, A., Neve, R. L., and Nestler, E. J. (2006). Sustained hippocampal chromatin regulation in a mouse model of depression and antidepressant action. Nat Neurosci 9, 519-525.

Vidal, M., and Gaber, R. F. (1991). RPD3 encodes a second factor required to achieve maximum positive and negative transcriptional states in Saccharomyces cerevisiae. Mol Cell Biol 11, 6317-6327.

Vincent, I., Rosado, M., and Davies, P. (1996). Mitotic mechanisms in Alzheimer's disease? J Cell Biol 132, 413-425.

Wang, J., Liu, S., Fu, Y., Wang, J. H., and Lu, Y. (2003). Cdk5 activation induces hippocampal CA1 cell death by directly phosphorylating NMDA receptors. Nat Neurosci 6, 1039-1047.

Wen, Y., Yang, S. H., Liu, R., Perez, E. J., Brun-Zinkernagel, A. M., Koulen, P., and Simpkins, J. W. (2007). Cdk5 is involved in NFT-like tauopathy induced by transient cerebral ischemia in female rats. Biochim Biophys Acta 1772, 473-483.

Yamaguchi, M., Tonou-Fujimori, N., Komori, A., Maeda, R., Nojima, Y., Li, H., Okamoto, H., and Masai, I. (2005). Histone deacetylase 1 regulates retinal neurogenesis in zebrafish by suppressing Wnt and Notch signaling pathways. Development 132, 3027-3043.

Yang, Y., Geldmacher, D. S., and Herrup, K. (2001). DNA replication precedes neuronal cell death in Alzheimer's disease. J Neurosci 21, 2661-2668.

Zhu, D. Y., Lau, L., Liu, S. H., Wei, J. S., and Lu, Y. M. (2004). Activation of cAMP-response-element-binding protein (CREB) after focal cerebral ischemia stimulates neurogenesis in the adult dentate gyms. Proc Natl Acad Sci U S A 101, 9453-9457.

### Equivalents

The foregoing written specification is considered to be sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention.

We claim:

1. A method for treating a neurological disorder in a subject, the method comprising administering to a subject in need of treatment for a neurological disorder a therapeutically effective amount of an HDAC1 (Histone deacetylase 1) activator to treat the neurological disorder.

2. The method of claim 1, wherein the neurological disorder is Alzheimer's disease.

3. The method of claim 1, wherein the neurological disorder is Parkinson's disease.

4. The method of claim 1, wherein the neurological disorder is Huntington's disease.

5. The method of claim 1, wherein the neurological disorder is ALS (Amyotrophic Lateral Sclerosis).

6. The method of claim 1, wherein the neurological disorder is traumatic brain injury.

7. The method of claim 1, wherein the neurological disorder is ischemic brain injury.

8. The method of claim 1, wherein the HDAC1 activator is an iron chelator.

9. The method of claim 8, wherein the iron chelator is deferoxamine.

10. The method of claim 1, wherein the HDAC1 activator is a flavonoid.

11. The method of claim 10, wherein the flavonoid is ginkgetin K.

12. The method of claim 1, wherein the HDAC1 activator is Chembridge 5104434.

13. The method of claim 1, wherein the HDAC1 activator is gambogic acid.

14. The method of claim 1, wherein the HDAC1 activator is sciadopilysin.

15. The method of claim 1, wherein the HDAC1 activator is tetrahydrogamboic acid.

16. The method of claim 1, wherein the HDAC1 activator is TAM-11.

17. The method of claim 1, wherein the HDAC1 activator is of the formula: 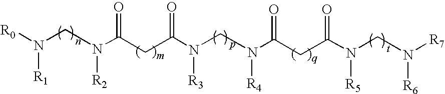
wherein
n is an integer between 1 and 6, inclusive;
m is an integer between 1 and 6, inclusive;
p is an integer between 1 and 6, inclusive;
q is an integer between 1 and 6, inclusive;
t is an integer between 1 and 6, inclusive;
R₀ is hydrogen, hydroxyl, acyl, or a nitrogen protecting group;
R₁ is hydrogen, hydroxyl, acyl, or a nitrogen protecting group;
R₂ is hydrogen, hydroxyl, acyl, or a nitrogen protecting group;
R₃ is hydrogen, hydroxyl, acyl, or a nitrogen protecting group;
R₄ is hydrogen, hydroxyl, acyl, or a nitrogen protecting group;
R₅ is hydrogen, hydroxyl, acyl, or a nitrogen protecting group;
R₆ is hydrogen, hydroxyl, acyl, or a nitrogen protecting group;
R₇ is hydrogen, hydroxyl, acyl, or a nitrogen protecting group; and pharmaceutically acceptable salts thereof.

18. The method of claim 1, wherein the HDAC1 activator is a catechol-containing compound of the formula: 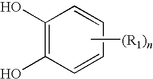
wherein
n is an integer between 1 and 4, inclusive;
each of R₁ is independently hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{A}; —C(═O)R_{A}; —CO₂R_{A}; —CN; —SCN; —SR_{A}; —SOR_{A}; —SO₂R_{A}; —NO₂; —N₃; —N(R_{A})₂; —NHC(═O)R_{A}; —NR_{A}C(═O)N(R_{A})₂; —OC(═O)OR_{A}; —OC(═O)R_{A}; —OC(═O)N(R_{A})₂; —NR_{A}C(═O)OR_{A}; or —C(R_{A})₃; wherein each occurrence of R_{A} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety; and pharmaceutically acceptable salts thereof.

19. The method of claim 18, wherein the HDAC1 activator is selected from the group consisting of levonordefrin 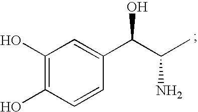
methyldopa (L, -) 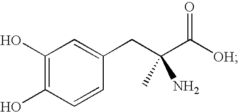
and R(+)-SKF-81297

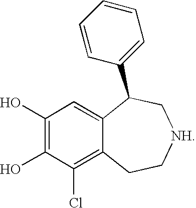

20. The method of claim 1, wherein the HDAC1 activator is of the formula:
wherein 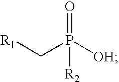
R₁ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{A}; —C(═O)R_{A}; —CO₂R_{A}; —CN; —SCN; —SR_{A}; —SOR_{A}; —SO₂R_{A}; —NO₂; —N₃; —N(R_{A})₂; —NHC(═O)R_{A}; —NR_{A}C(═O)N(R_{A})₂; —OC(═O)OR_{A}; —OC(═O)R_{A}; —OC(═O)N(R_{A})₂; —NR_{A}C(═O)OR_{A}; or —C(R_{A})₃; wherein each occurrence of R_{A} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;
R₂ is cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{B}; —OH; or —C(R_{B})₃; wherein each occurrence of R_{B} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety; and
pharmaceutically acceptable salts thereof

21. The method of claim 20, wherein the HDAC1 activator is selected from the group consisting of LY 235959 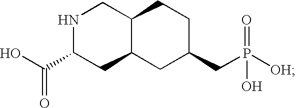
SK&F97541 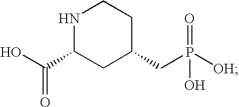
SK&F 97541 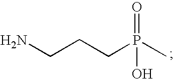
and etidronic acid

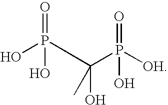

22. The method of claim 1, wherein the HDAC1 activator is of the formula: 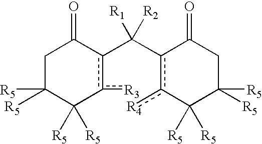
wherein
each [symbol] is independently a single or double bond;
each of R₁ and R₂ is independently hydrogen; cyclic or acyclic, branched or unbranched, substituted or unsubstituted aliphatic; cyclic or acyclic, substituted or unsubstituted, branched or unbranched heteroaliphatic; substituted or unsubstituted, branched or unbranched acyl; substituted or unsubstituted aryl, substituted or unsubstituted, branched or unbranched heteroaryl; —OR_{A}; —C(═O)R_{A}; —CO₂R_{A}; —CN; —SCN; —SR_{A}; —SOR_{A}; —SO₂R_{A}; —NO₂; —N₃; —N(R_{A})₂; —NHC(═O)R_{A}; —NR_{A}C(═O)N(R_{A})₂; —OC(═O)OR_{A}; —OC(═O)R_{A}; —OC(═O)N(R_{A})₂; —NR_{A}C(═O)OR_{A}; or —C(R_{A})₃; wherein each occurrence of R_{A} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;
each of R₃, and R₄ is independently —OH, alkoxy, —Oacyl, ═O, or wherein R₃ and R₄ are taken together to form a cyclic structure;
each of R₅ is independently hydrogen; cyclic or acyclic, branched or unbranched, substituted or unsubstituted aliphatic; and pharmaceutically acceptable salts thereof.

23. The method of claim 1, wherein the HDAC1 activator is of the formula: 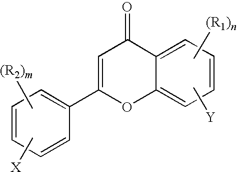
wherein
n is an integer between 0 and 4, inclusive;
m is an integer between 0 and 5, inclusive;
each of R₁ and R₂ is independently —OH; alkoxy; —Oacyl; —OAc; —OP_{G}; substituted or unsubstituted aryl; wherein either R₁ or R₂ can be a second HDAC1 activator moiety; and pharmaceutically acceptable salts thereof.

24. The method of claim 1, wherein the HDAC1 activator is of the formula: 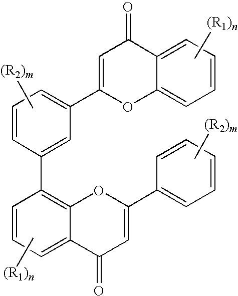
wherein
n is an integer between 0 and 4, inclusive;
m is an integer between 0 and 4, inclusive;
each of R₁ and R₂ is independently —OH; alkoxy; —Oacyl; —OAc; —OP_{G}; substituted or unsubstituted aryl; and pharmaceutically acceptable salts thereof.

25. The method of claim 1, wherein the HDAC1 activator is of the formula: 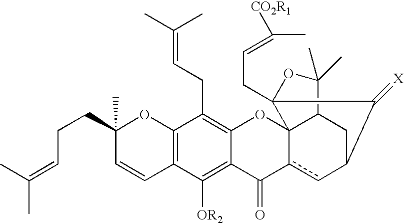
wherein [symbol]
is independently a single or double bond;
R₁ is hydrogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched aryl; substituted or unsubstituted, branched or unbranched heteroaryl;
R₂ is hydrogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —C(═O)R_{B}; —CO₂R_{B}; or —C(R_{B})₃; wherein each occurrence of R_{B} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;
X is ═O, 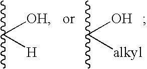
and pharmaceutically acceptable salts thereof

26. A method for protecting a subject against neuronal damage, the method comprising administering to a subject in need of protection against neuronal damage a therapeutically effective amount of an HDAC1 (Histone deacetylase 1) activator to protect against neuronal damage.

27. The method of claim 26, wherein the neuronal damage is ischemic brain damage.

28. The method of claim 26, wherein the neuronal damage is stroke.

29.-46. (canceled)

47. A method for increasing HDAC1 (Histone deacetylase 1) activity in a cell, the method comprising contacting the cell with an HDAC1 activator.

48. The method of claim 47, wherein increasing HDAC1 activity comprises increasing the deacetylase activity of HDAC1.

49. The method of claim 47, wherein increasing the HDAC1 activity comprises increasing the expression level of HDAC1.

50. The method of claim 47, wherein the cell is in a subject.

51.-68. (canceled)

69. A compound of the formula: 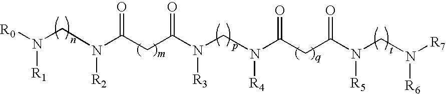
wherein
n is an integer between 1 and 6, inclusive;
m is an integer between 1 and 6, inclusive;
p is an integer between 1 and 6, inclusive;
q is an integer between 1 and 6, inclusive;
t is an integer between 1 and 6, inclusive;
R₀ is hydrogen, hydroxyl, acyl, or a nitrogen protecting group;
R₁ is hydrogen, hydroxyl, acyl, or a nitrogen protecting group;
R₂ is hydrogen, hydroxyl, acyl, or a nitrogen protecting group;
R₃ is hydrogen, hydroxyl, acyl, or a nitrogen protecting group;
R₄ is hydrogen, hydroxyl, acyl, or a nitrogen protecting group;
R₅ is hydrogen, hydroxyl, acyl, or a nitrogen protecting group;
R₆ is hydrogen, hydroxyl, acyl, or a nitrogen protecting group;
R₇ is hydrogen, hydroxyl, acyl, or a nitrogen protecting group; and a pharmaceutically acceptable salt thereof

70. A compound of the formula: 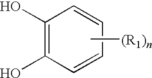
wherein
n is an integer between 1 and 4, inclusive;
each of R₁ is independently hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{A}; —C(═O)R_{A}; —CO₂R_{A}; —CN; —SCN; —SR_{A}; —SOR_{A}; —SO₂R_{A}; —NO₂; —N₃; —N(R_{A})₂; —NHC(═O)R_{A}; —NR_{A}C(═O)N(R_{A})₂; —OC(═O)OR_{A}; —OC(═O)R_{A}; —OC(═O)N(R_{A})₂; —NR_{A}C(═O)OR_{A}; or —C(R_{A})₃; wherein each occurrence of R_{A} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety; and pharmaceutically acceptable salts thereof.

71. The compound of claim 70, wherein the HDAC1 activator is selected from the group consisting of levonordefrin 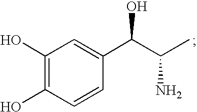
methyldopa (L, -) 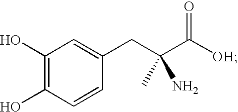
and R(+)-SKF-81297

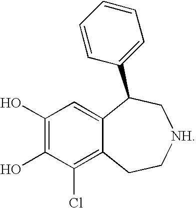

72. A compound of the formula: 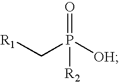
wherein
R₁ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{A}; —C(═O)R_{A}; —CO₂R_{A}; —CN; —SCN; —SR_{A}; —SOR_{A}; —SO₂R_{A}; —NO₂; —N₃; —N(R_{A})₂; —NHC(═O)R_{A}; —NR_{A}C(═O)N(R_{A})₂; —OC(═O)OR_{A}; —OC(═O)R_{A}; —OC(═O)N(R_{A})₂; —NR_{A}C(═O)OR_{A}; or —C(R_{A})₃; wherein each occurrence of R_{A} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;
R₂ is cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{B}; —OH; or —C(R_{B})₃; wherein each occurrence of R_{B} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety; and pharmaceutically acceptable salts thereof.

73. The compound of claim 72, wherein the HDAC1 activator is selected from the group consisting of LY 235959 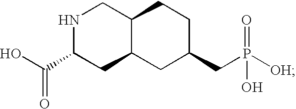
CGS 19755 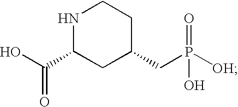
SK&F97541 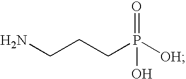
and etidronic acid

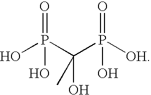

74. A compound of the formula: 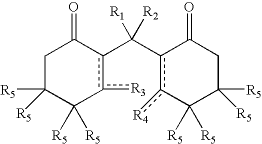
wherein
each [symbol] is independently a single or double bond;
each of R₁ and R₂ is independently hydrogen; cyclic or acyclic, branched or unbranched, substituted or unsubstituted aliphatic; cyclic or acyclic, substituted or unsubstituted, branched or unbranched heteroaliphatic; substituted or unsubstituted, branched or unbranched acyl; substituted or unsubstituted aryl, substituted or unsubstituted, branched or unbranched heteroaryl; —OR_{A}; —C(═O)R_{A}; —CO₂R_{A}; —CN; —SCN; —SR_{A}; —SOR_{A}; —SO₂R_{A}; —NO₂; —N₃; —N(R_{A})₂; —NHC(═O)R_{A}; —NR_{A}C(═O)N(R_{A})₂; —OC(═O)OR_{A}; —OC(═O)R_{A}; —OC(═O)N(R_{A})₂; —NR_{A}C(═O)OR_{A}; or —C(R_{A})₃; wherein each occurrence of R_{A} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;
each of R₃, and R₄ is independently —OH, alkoxy, —Oacyl, ═O, or wherein R₃ and R₄ are taken together to form a cyclic structure;
each of R₅ is independently hydrogen; cyclic or acyclic, branched or unbranched, substituted or unsubstituted aliphatic; and pharmaceutically acceptable salts thereof.

75. A compound of the formula: 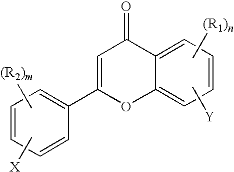
wherein
n is an integer between 0 and 4, inclusive;
m is an integer between 0 and 5, inclusive;
each of R₁ and R₂ is independently —OH; alkoxy; —Oacyl; —OAc; —OP_{G}; substituted or unsubstituted aryl; wherein either R₁ or R₂ can be a second HDAC1 activator moiety; and pharmaceutically acceptable salts thereof.

76. A compound of the formula: 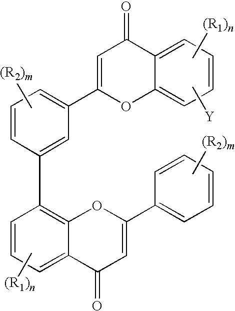
wherein
n is an integer between 0 and 4, inclusive;
m is an integer between 0 and 4, inclusive;
each of R₁ and R₂ is independently —OH; alkoxy; —Oacyl; —OAc; —OP_{G}; substituted or unsubstituted aryl; and pharmaceutically acceptable salts thereof.

77. A compound of the formula: 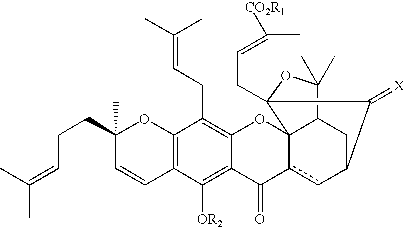
wherein [symbol]
is independently a single or double bond;
R₁ is hydrogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched aryl; substituted or unsubstituted, branched or unbranched heteroaryl;
R₂ is hydrogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —C(═O)R_{B}; —CO₂R_{B}; or —C(R_{B})₃; wherein each occurrence of R_{B} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;
X is ═O, 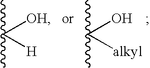
and pharmaceutically acceptable salts thereof.

78.-95. (canceled)
